(19) United States
(12) Patent Application Publication
(10) Pub. No.: US 2024/0417458 A1
(43) Pub. Date: Dec. 19, 2024

(54) METHODS FOR TREATING ANEMIA OF KIDNEY DISEASE

(71) Applicants: Disc Medicine, Inc., Watertown, MA (US); AbbVie Deutschland GmbH & Co. KG, Wiesbaden (DE); AbbVie Inc., North Chicago, IL (US)

(72) Inventors: Brian MacDonald, Bend, OR (US); John Quisel, Cambridge, MA (US); Will Savage, Newton Highlands, MA (US); Hua Yang, Acton, MA (US); Jonathan Yu, Cambridge, MA (US); Min Wu, Cambridge, MA (US); Maria Beconi, Cambridge, MA (US); Jennifer M. Perez, Worcester, MA (US); Bernhard Mueller, Hannover (DE); Andreas Popp, Sprockhovel (DE)

(73) Assignees: Disc Medicine, Inc., Watertown, MA (US); AbbVie Deutschland GmbH & Co. KG, Wiesbaden (DE); AbbVie Inc., North Chicago, IL (US)

(21) Appl. No.: 18/710,958

(22) PCT Filed: Nov. 16, 2022
(86) PCT No.: PCT/US2022/079987

§ 371 Date: May 16, 2024

Related U.S. Application Data
(60) Provisional application No. 63/419,648, filed on Oct. 26, 2022.
(60) Provisional application No. 63/280,594, filed on Nov. 17, 2021.

(51) Int. Cl. C07K 16/28; A61P 7/06

(52) U.S. Cl. CPC C07K 16/28; A61P 7/06; C07K 2317/24

(57) ABSTRACT

Aspects of the application provide hemojuvelin antagonists and methods of using the same in treating anemias of kidney disease and conditions associated with these anemias. Methods provided in the application relate to treatment of a subject having an anemia associated with chronic kidney disease and/or kidney disease in a subject that has a level of glomerular filtration rate lower than one or more thresholds.

### CROSS-REFERENCE TO RELATED APPLICATIONS

This application claims the benefit of the filing dates of U.S. Provisional Application No. 63/280,594, filed Nov. 17, 2021, and U.S. Provisional Application No. 63/419,648, filed Oct. 26, 2022, the entire contents of each of which are incorporated by reference.

### REFERENCE TO AN ELECTRONIC SEQUENCE LISTING

The contents of the electronic sequence listing (D084270006WO00-SEQ-EPG.xml; Size: 204,621 bytes; and Date of Creation: Nov. 16, 2022) is herein incorporated by reference in its entirety.

### BACKGROUND

Kidney disease is a condition characterized by abnormalities of kidney structure or function. Outcomes of kidney disease can include kidney failure, as well as complications of decreased kidney function and cardiovascular diseases. When kidney abnormalities persist for an extended period of time, the kidney disease may be classified as Chronic kidney disease (CKD). Cardiovascular mortality is the leading cause of death in CKD patients. As of 2017, the estimated prevalence of CKD worldwide was 9.1% of the world's population, and this prevalence is rising. CKD most commonly typically presents in people aged 65 years or older. The stage of CKD is defined according to the level of glomerular filtration rate (GFR).

Many kidney disease patients have or develop anemia. Left untreated, anemia negatively impacts cardiac function, increases the risk of blood transfusion and significantly impairs quality of life. In some cases, untreated anemia can be fatal, particularly in the context of CKD. A new class of drugs has emerged for treatment of anemia of CKD, referred to as hypoxia inducible factor prolyl hydroxylase inhibitor (HIF-PHI) drugs. These drugs are designed to help prompt the body to make red blood cells. No HIF-PHI has been approved by the FDA to date. Thus, no HIF-PHI has been demonstrated to be safe and effective against anemia of CKD in US clinical trials to date. A major efficacy concern with respect to HIF-PHI drugs is an overcorrection of hemoglobin (Hb) levels in serum, which can lead to adverse cardiovascular outcomes. Thus, there is a need for a safe and effective treatment of anemia associated with kidney disease (e.g., CKD).

### SUMMARY

Aspects of the present disclosure relate to methods for treating subjects having anemia that involve the use of hepcidin antagonists (e.g., hemojuvelin antagonists). For example, in some embodiments, methods are provided herein that involve administration of a hemojuvelin antagonist to treat anemia in a subject having kidney disease. Further aspects of the disclosure provide methods for treating anemia in a subject having chronic kidney disease (CKD). In some embodiments, disclosed methods provide administration of a hemojuvelin antagonist to a subject having (or exhibiting) certain levels of glomerular filtration rate (GFR), as measured or estimated by a physician. In some embodiments, methods provided herein involve subcutaneous administration of a hemojuvelin antagonist (e.g., an anti-hemojuvelin antagonist antibody) to treat the anemia, and thus, are advantageous for administration outside of a hospital or similar healthcare setting where intravenous and similar administrations or procedures are typically performed. For example, in some embodiments, subcutaneous administration-based methods provided herein may be performed in a physician's office or as a self-administration by a subject or patient. Accordingly, aspects of the present disclosure are useful for treating anemia in subjects having kidney disease in which the subject is not undergoing a therapy (such as dialysis therapy, IV iron therapy, and other similar therapies) that involves one or more visit(s) to a hospital or similar healthcare setting where intravenous therapies can be readily administered.

Further aspects of the present disclosure relate to treating a subject with anemia wherein the subject is identified as having a level of glomerular filtration rate (GFR) of less than 90 mL/min per 1.73 m², by administering to the subject an effective amount of a hemojuvelin antagonist. Certain aspects of this disclosure relate to treatment of anemias that are caused, at least in part, by a nutritional iron deficiency, iron deficiency due to blood loss, an intrinsic red blood cell disorder, hemolysis, inflammation, functional iron deficiency, or any combination of the foregoing. In some embodiments, the subject may have chronic kidney disease. Accordingly, in some embodiments, the level of glomerular filtration rate in the subject has persisted for at least three months. In some embodiments, the anemia is associated with kidney damage of the subject. The kidney damage may have been present in the subject for at least three months. In some embodiments, the subject has a chronic kidney disease that is a non-dialysis dependent chronic kidney disease. Likewise, in some embodiments, the subject is not undergoing dialysis therapy.

Anemia is a common complication in patients with CKD and has been associated with multiple adverse outcomes in this population. Increased hepcidin is believed to be a central contributor to the development of anemia because of both reduced clearance and increased synthesis of hepcidin. Treatment with hepcidin lowering drugs has been demonstrated to increase iron availability from systemic iron stores and to increase hemoglobin (Sheetz et al, Br J Clin Pharmacol. 2019; 85:935-948, which is incorporated by reference herein). By downregulating hepcidin, it is hypothesized that an anti-hemojuvelin antibody (anti-HJV Ab) will have beneficial effects in treating anemia in the CKD patients because it will make iron available for improved erythropoiesis.

Accordingly, in some embodiments, methods provided herein are useful for treating subjects having anemia associated with kidney disease so as to decrease hepcidin levels or activity. In some embodiments, the subject may experience an improvement in iron uptake from the gastrointestinal system (i.e., from diet). In some embodiments, the subject may experience a restoration, either partial or complete, of iron levels.

In some embodiments, methods provided herein are useful for treating subjects who have low TSAT levels (e.g., below 20%, below 30%, below 40%, or below 50%) and/or low ferritin levels (e.g., ferritin below ≤50 ng/ml, ≤100 ng/ml, or ≤300 ng/ml). In some embodiments, methods provided herein are useful for treating subjects who exhibit low hemoglobin levels (e.g., Hb levels below 11 g/dL, 10 g/dL, or 9 g/dL). In some embodiments, methods provided herein are useful for treating subjects who exhibit normal to relatively high hepcidin levels. In some embodiments, the subjects further exhibit renal impairment, which may be chronic, with a glomerular filtration rate in the range of 15 mL/min to just below 60 (e.g., 59) mL/min per 1.73 m².

In some embodiments, the hemojuvelin antagonist is an anti-hemojuvelin antibody. In some embodiments, the anti-hemojuvelin antibody preferentially binds RGMc versus RGMa and RGMb. In some embodiments, the anti-hemojuvelin antibody binds RGMc with an equilibrium dissociation constant (K_{D}) less than 100 nM. In some embodiments, the anti-HJV antibody is an anti-HJV antibody in Table 1.

In some embodiments, the anti-hemojuvelin antibody comprises: (a) a variable heavy chain region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and/or (b) a variable light chain region comprising a CDR1 comprising an amino acid sequence of SEQ ID NO: 17, a CDR2 comprising an amino acid sequence of SEQ ID NO: 5, and a CDR3 comprising an amino acid sequence of SEQ ID NO: 27. In exemplary embodiments, the antibody comprises a HC CDR1 of SEQ ID NO: 1, a HC CDR2 of SEQ ID NO: 2, a HC CDR3 of SEQ ID NO: 3; an LC CDR1 of SEQ ID NO: 17, a LC CDR2 of SEQ ID NO: 5, and a LC CDR3 of SEQ ID NO: 27. In some embodiments, the anti-hemojuvelin antibody is hHA-008. In some embodiments, the anti-hemojuvelin antibody is hHA-008-QL.

In some embodiments, the antibody comprises a V_{H} comprising an amino acid sequence of SEQ ID NO: 38, and a VL comprising an amino acid sequence of SEQ ID NO: 39. The antibody may be selected from the group consisting of a full-length IgG, a Fab fragment, a F(ab′) fragment, a F(ab′) 2 fragment, a scFv, and a Fv. In some embodiments, the antibody is a full-length IgG comprising a heavy chain constant region of the isotype IgG1. IgG2, IgG3, or IgG4.

Accordingly, in some embodiments, provided are methods of treating a subject having anemia associated with a chronic kidney disease by administering an anti-hemojuvelin antibody that comprises a V_{H} comprising an amino acid sequence of SEQ ID NO: 38, and a VL comprising an amino acid sequence of SEQ ID NO: 39.

In some embodiments, the antibody comprises a heavy chain comprising an amino acid sequence of SEQ ID NO: 61, and a light chain comprising an amino acid sequence of SEQ ID NO: 62. In other embodiments, the antibody comprises a heavy chain comprising an amino acid sequence of SEQ ID NO: 63, and a light chain comprising an amino acid sequence of SEQ ID NO: 62.

In some embodiments, the anti-hemojuvelin antibody is administered to a subject in need thereof in an amount between 0.1 and 0.8 mg/kg of subject. In some embodiments, the antibody is administered in a dose of between about 7 mg and 56 mg, such as 56 mg. Any of these doses may be administered monthly or semi-monthly.

In some embodiments, the subject is identified prior to the treatment as having high hepcidin levels. In some embodiments, the subject is identified as having a functional iron deficiency. In some embodiments, the subject is identified as exhibiting inflammation and/or iron-restricted erythropoiesis. In some embodiments, the subject is a human. In some embodiments, the subject has a nutritional iron deficiency. In some embodiments, the subject does not have a nutritional iron deficiency.

In some embodiments, the subject has serum ferritin levels above 100 μg/L (100 ng/ml). In other embodiments, the subject has serum ferritin levels lower than 100 μg/L. In some embodiments, the subject has reticulocyte hemoglobin content less than 26 pg/cell. In some embodiments, the subject has a transferrin saturation level less than 50%, 30%, or 25%. In some embodiments, the subject has hepatic iron levels higher than 2000 μg/g dry weight. In some embodiments, the subject has serum iron levels in a range of less than 50 μg/dL. In some embodiments, the subject has a total iron binding capacity in a range of less than 400 μg/dL. In some embodiments, the subject has hepcidin levels in a range of more than 55 ng/ml. In some embodiments, the subject has IL-6 levels of more than 1.8 pg/mL. In some embodiments, the subject has serum creatinine values of more than 2 mg/dL.

In some embodiments, the subject has been identified as having hemoglobin levels in the range of 1.5 to 2.0 g/dL or 2.0 to 4.0 g/dL or more below normal hemoglobin levels. In some embodiments, the subject presents with a serum hemoglobin level of less than 10 g/dL. In some embodiments, the subject presents with a serum hemoglobin level of less than 8 g/dL. In some embodiments, the administration of the hepcidin antagonist increases hemoglobin level at least 1 g/dL from baseline.

In some embodiments, methods of treating a subject further comprise administering to the subject one or more additional therapeutic agents, such as a growth differentiation factor (GDF) trap, an erythropoiesis stimulating agent (ESA), oral iron, intravenous iron (IV iron), a hypoxia inducible factor prolyl hydroxylase inhibitor (HIF-PHI), or a red blood cell transfusion. In some embodiments, a GDF trap, such as sotatercept or luspatercept, is administered. In some embodiments, an ESA such as erythropoietin (EPO) is administered. In some embodiments, the additional therapeutic agent is oral iron. In particular embodiments, the additional therapeutic agent is oral iron in a dose of about 30 mg, biweekly (twice a week). In some embodiments, the additional therapeutic agent is an HIF-PHI.

In some embodiments, methods provided herein that decrease hepcidin levels or activity may be combined with oral iron therapy to facilitate restoration of iron levels. Thus, in some embodiments, such methods provided herein are useful for treating anemic subjects who are experiencing intolerance to oral iron or an unsatisfactory response to oral iron.

Accordingly, combination therapies are provided comprising any of the disclosed hemojuvelin antagonists and one or more of a growth differentiation factor (GDF) trap, an erythropoiesis stimulating agent (ESA), oral iron, IV iron (such as MonoFerric®), a hypoxia inducible factor prolyl hydroxylase inhibitor (HIF-PHI), and a red blood cell transfusion.

In some embodiments, administration of any of the disclosed anti-hemojuvelin antibodies results in, or provides, an increase in hemoglobin levels in the subject at least 2, 4, 6, 8, 10, 11, 12, 13, 14, 15, 16, 17, 18, 19, 20, or more than 20 g/dL relative to an untreated subject. In some embodiments, the administration results in an increase in hemoglobin level in the subject of about 17 g/dL. Administration may result in an increase in reticulocyte hemoglobin (Ret-HGB) levels in the subject by at least 0.2, 0.3, 0.4, 0.5, 0.6, 0.7, 0.75, 0.8, 0.85, 0.9, 0.95, 1.0, or more than 1.0 pg relative to an untreated subject. In some embodiments, administration results in an increase in serum iron levels in the subject, relative to an untreated subject, of about 25, 27.5 30, 32.5, 35, 38.5, 40, or 45 μmol/L. Administration may result in an increase in red blood cell (RBC) counts in the subject, relative to an untreated subject, of about 1, 2, 2.5, 3, 4, 5, 6, 7, 7.5, 8, 9, or 10×10⁵ cells/μL. Administration may result in a decrease in reticulocyte (Ret) counts in the subject, relative to an untreated subject, of about 90, 100, 100, 120 or 125×10⁹ cells/L.

The disclosed methods may be particularly useful for subcutaneous, intravenous, or intramuscular administration of the hemojuvelin antagonist (e.g., anti-hemojuvelin antibody). Accordingly, the disclosed methods may comprise a step of administration of the antagonist by subcutaneous, intravenous, or intramuscular injection. In exemplary embodiments, the step of administering is by subcutaneous injection. In some embodiments, the subcutaneous injection is performed as a self-administration.

The foregoing and other aspects, implementations, acts, functionalities, features and, embodiments of the present teachings can be more fully understood from the following description in conjunction with the accompanying drawings.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIGS. 1A-1G are graphs showing generation and characterization of anti-HJV antibodies. FIG. 1A shows a schematic process of generation of the anti-hemojuvelin antibody, humanization, and affinity maturation. FIGS. 1B-1G shows the sensorgrams by BIAcore analysis of antibodies HA, hHA-004, hHA-008, hHA-009 and hHA-011.

FIGS. 2A-2C are graphs showing the BMP reporter gene assay for anti-HJV antibodies. FIG. 2A shows the general principle of HJV BMP reporter assay. FIG. 2B shows the effect of anti-HJV antibodies in inhibiting RGMc BMP signaling. FIG. 2C shows the effect of anti-HJV antibodies in inhibiting RGMa BMP signaling.

FIG. 3 is a graph showing anti-HJV antibodies non-specific binding to HEK293 cells. Bars from left to right in each group: 100 μg/ml, 10 μg/ml, and 1 μg/ml.

FIGS. 4A-4C are schematic illustrations showing the structure and designs of hHA-008 and hHA-008-QL. FIG. 4A shows the structure of hHA-008. FIG. 4B shows the structure of hHA-008-QL. FIG. 4C shows a comparison in antibody structure between hHA-008 and hHA-008-QL.

FIGS. 5A-5B are graphs showing the CD4+ T cell response peripheral blood mononuclear cells (PBMCs) challenged with hHA-008 or hHA-008-QL.

FIGS. 6A-6C are graphs showing PK/PD analysis of hHA-008 in rats. FIG. 6A shows maximal effect of hHA-008 measured by TSAT % occurred between 4-8 days post treatment. FIG. 6B shows hHA-008 reached maximum effect as measured by TSAT % about 1-4 days after injection in female cynos. FIG. 6C shows hHA-008 reached maximum effect as measured by TSAT % about 1-4 days after injection in male cynos, but one of the males did not respond to hHA-008 treatment.

FIGS. 7A-7F shows PK/PD correlation in Cynos with single IV dose of 6 mpk. FIG. 7A shows the maximum TSAT % increase occurred 1-4 days after injection (Tₘₐₓ=1-4 days), and one of the animals tested had a drastic decline of TSAT around day 34. FIG. 7B shows plasma hepcidin-25 concentration changes over time after hHA-008 injection. FIG. 7C shows plasma hHA-008 concentration changes over time after hHA-008 injection. FIGS. 7D-7F show that hHA-008 had robust PK/PD correlation of PK (plasma antibody concentration) to TSAT % and plasma hepcidin-25 concentration. The results of each tested Cyno are shown in FIG. 7D (Cyno 1), FIG. 7E (Cyno 2), and FIG. 7F (Cyno 3).

FIGS. 8A-8C show that hHA-008 antibody modulates TSAT % in a dose-dependent manner. FIG. 8A shows TSAT % and hHA008 concentrations after animals were treated with either 0 (vehicle control) or 0.6 mpk hHA-008. FIG. 8B shows TSAT % and hHA-008 concentrations after animals were treated with either 0 (vehicle control) or 3 mpk hHA-008. FIG. 8C shows TSAT % and hHA-008 concentrations after animals were treated with either 0 (vehicle control) or 60 mpk hHA-008.

FIGS. 9A-9C are graphs showing the PK/PD comparison between hHA-008 and hHA-008-QL. FIG. 9A shows TSAT % changes over time in Cynos post treatment of hHA-008 or hHA-008-QL. FIG. 9A shows TSAT % changes over time in Cynos post treatment of hHA-008 or hHA-008-QL. FIG. 9B shows plasma concentration of the antibodies over time in Cynos post treatment of hHA-008 or hHA-008-QL. FIG. 9C shows a time course of decline of plasma concentration of hHA-008 and hHA-008-QL.

FIGS. 10A-10D are graphs showing binding of FcRn of hHA-008 and hHA-008-QL at pH 6.0 or 7.4. FIGS. 10A-10B shows binding of FcRn of hHA-008 and hHA-008-QL at pH 6.0. FIGS. 10C-10D shows binding of FcRn of hHA-008 and hHA-008-QL at pH 7.4. X axis: Time. Y Axis: Response.

FIG. 11 depicts a myeloproliferation cycle characteristic of certain high hepcidin disorders.

FIG. 12 depicts the hepcidin stimulatory pathway and the physiological regulation of iron homeostasis by hepcidin.

FIG. 13 is a graph showing that IL-6 induces hepcidin expression in Cynos, and hHA-008 treatment prevents inflammation-induced (IL 6) hepcidin-25 increase in a dose-dependent manner.

FIG. 14 shows hHA-008 interacts with amino acids 170-183 (SSPMALGANATATR (SEQ ID NO: 121)) on 3720-RG-050. The interaction happens on amino acids 170, 171, 180, 182, 183 on 3720-RG-050.

FIGS. 15A-15J show the interaction of 3720-RG-050 and hHA-008. A 3720-RG-050 PDB structure was generated by homology using Swiss Model software. 3720-RG-050 amino acids 170-183 (SSPMALGANATATR (SEQ ID NO: 121)) are shown in FIGS. 15A, 15B, 15C, 15D, 15E: ribbon/surface representation of front view (FIG. 15A); back view (FIG. 15B), side view 1 (FIG. 15C), side view 2 (FIG. 15D) and top view (FIG. 15E). FIGS. 15F, FIG. 15G, FIG. 15H, FIG. 15I, FIG. 15J: ribbon representation of front view (FIG. 15F); back view (FIG. 15G), side view 1 (FIG. 15H), side view 2 (FIG. 15I) and top view (FIG. 15J).

FIG. 16 shows hHA-008-QL interacts with amino acids 169-182 (TSSPMALGANATAT (SEQ ID NO: 122)) and 289-300 (SQRLSRSERNRR (SEQ ID NO: 127)) of 3720-RG-050. The interaction happens on amino acids 169, 171, 180, 182; 289, 293, 294, 295, 297, 300 on 3720-RG-050.

FIGS. 17A-17J show the interaction 3720-RG-050/hHA-008-QL. A 3720-RG-050 PDB structure was generated by homology using Swiss Model software. 3720-RG-050 amino acids 169-182 (TSSPMALGANATAT (SEQ ID NO: 122)) and 289-291 (SQR) are shown in FIGS. 17A, 17B, 17C, 17D, 17E: ribbon/surface representation of front view (FIG. 17A); back view (FIG. 17B), side view 1 (FIG. 17C), side view 2 (FIG. 17D) and top view (FIG. 17E). FIGS. 17F, 17G, 17H, 17I, 17J: ribbon representation of front view (FIG. 17F); back view (FIG. 17G), side view 1 (FIG. 17H), side view 2 (FIG. 17I) and top view (FIG. 17J).

FIG. 18 shows that hHA-008 was effective in preventing IL-6-induced serum iron suppression in a dose-dependent manner in cynomolgus monkeys.

FIG. 19 shows that decline in PD response (e.g., Hepcidin-25 concentration and TSAT %) was consistent with the decrease of hHA-008 serum concentration (FIG. 19) after subcutaneous administration of hHA-008 to Sprague-Dawley Rats.

FIGS. 20A-20D show PK/PD analysis in cynomolgus monkeys after subcutaneous administration of hHA-008. FIG. 20A shows serum concentration-time profiles became indistinguishable between SC injection and IV injection 4 days after administration. FIGS. 20B-20D show the return of serum iron to baseline levels was consistent with the decline in hHA-008 serum concentrations after 0.3 mpk. 0.6 mpk and 1 mpk injection of hHA-008 either by subcutaneous injection or intravenous injection.

FIGS. 21A and 21B illustrate functional iron deficiency in chronic kidney disease (CKD). FIG. 21A depicts functional iron deficiency in the context of CKD. FIG. 21B shows pathways of CKD and inflammation leading to anemia.

FIG. 22 depicts the hepcidin stimulatory pathway and the physiological regulation of iron homeostasis by hepcidin.

FIGS. 23A-23G illustrate the role of hepcidin in functional iron deficiency (FID) and examples of regulating hepcidin level by hepcidin antagonists. FIG. 23A depicts the mechanism of functional iron deficiency. FIG. 23B shows that functional iron deficiency is a common feature of anemia of inflammation and chronic diseases including chronic kidney disease (CKD). FIG. 23C shows that functional iron deficiency is associated with high iron level and high hepcidin level. FIG. 23D is a schematic illustration of decreasing hepcidin level to normal by using hepcidin antagonists for treatment of iron restriction diseases. FIG. 23E depicts using anti-HJV antibody as one example to inhibit the HJV induced BMP signaling pathway to reduce hepcidin to normal level. FIG. 23F shows that Matriptase-2 negatively regulates hepcidin by cleaving membrane bound HJV. FIG. 23G depicts examples of possible hepcidin antagonists for regulation of hepcidin level.

FIG. 24 shows serum Fe concentrations after subcutaneous (SC) hHA-008 administrations to male and female rats as either a single 6 mg/kg or 30 mg/kg dose.

FIG. 25 shows serum Fe measured after either 6 mg/kg or 30 mg/kg given to rats as an intravenous (IV) or subcutaneous (SC) dose.

FIG. 26 shows hHA-008 pharmacokinetics after IV and SC administrations to cynomolgus monkeys—0.3, 0.6, 1.0, and 6.0 mg/kg dose levels.

FIGS. 27A-27D show serum hepcidin-25 concentrations after IV and SC hHA-008 administrations—0.3, 0.6, 1.0, and 6.0 mg/kg dose levels.

FIG. 28 shows hHA-008 pharmacokinetics after IV and SC administrations to cynomolgus monkeys—0.3, 0.6, 1.0, and 6.0 mg/kg dose levels.

FIGS. 29A-29D show serum Fe concentrations after IV and SC hHA-008 administrations—0.3, 0.6, 1.0, and 6.0 mg/kg dose levels.

FIGS. 30A-30D show transferrin saturation % (TSAT %) after IV and SC hHA-008 administrations—0.3, 0.6, 1.0, and 6.0 mg/kg dose levels.

FIG. 31 is a schematic that shows the design of an in vivo study to evaluate the effects of treatment with a lead anti-hemojuvelin antibody (Anti-HJV Ab) in a rat model of anemia of CKD.

FIG. 32 shows the metabolic effects of the adenine diet-induced kidney injury and anemia in the CKD rat model, following administration of an empty vehicle.

FIG. 33 is a series of line plots showing the variations in levels of HAMP (hepcidin gene) mRNA expression, hepcidin-25 and iron in serum of the rat models following anti-HJV Ab administration. This figure indicates that Anti-HJV Ab decreased hepcidin and increased serum iron levels in CKD rats over the course of the treatment.

FIG. 34 is a series of line plots showing the effects of Anti-HJV Ab on reticulocyte hemoglobin, mean corpuscular hemoglobin (MCH), hemoglobin (HGB), reticulocyte counts, red blood cell (RBC) counts, and mean corpuscular volume (MCV) over the course of the treatment.

### DETAILED DESCRIPTION

According to some aspects, the disclosure provides methods of administering an effective amount of a hemojuvelin antagonist that are effective for inhibiting hepcidin function and/or reducing hepcidin expression. In some embodiments, these methods are also particularly useful for the treatment of anemia in a subject that is identified as having a level of glomerular filtration rate (GFR) of less than 90 mL/min per 1.73 m². In some embodiments, these methods are particularly useful for the treatment of anemia of kidney disease (such as CKD) and/or one or more symptoms or complications thereof. Accordingly, in some embodiments, these methods may be used to treat a subject having kidney disease wherein the disease is chronic or not chronic. Accordingly, in related aspects, the disclosure provides compositions and methods for treating anemias that may be associated with chronic kidney disease.

A frequent complication of CKD is anemia caused by insufficient erythropoietin (EPO) production by the damaged kidneys. As EPO production decreases the bone marrow responds by decreasing RBC counts. Other factors in CKD patients that lead to anemia are iron deficiency, blood loss, and shortened red blood cell (RBC) survival. In some embodiments, to classify severe CKD patients as anemic or non-anemic the following criteria are commonly applied: non-anemic (≥12/≥13 g/dL hemoglobin (Hb) in women/men); anemia grade 1 (10-12/13 g/dL Hb in women/men); grade 2 (8-10 g/dL Hb); and grade 3+(<8 g/dL Hb). See Toft et al., J Nephrol. 2020 February; 33 (1): 147-156, which is herein incorporated by reference.

The etiology of anemia associated with CKD is multifactorial, arising from any one of the following causes: deficiency of EPO, impaired iron absorption (functional iron deficiency), inability to utilize stored iron, or shortened red blood cell survival. Subjects suffering from anemia associated with CKD typically present with reduced endogenous EPO levels and/or functional iron deficiency. Functional iron deficiency, which presents at low transferrin saturation (TSAT) levels often leads to EPO resistance. These subjects also typically present with reduced hepcidin levels. Hepcidin(−25) is a 25-amino acid protein that is filtered from the blood through the kidneys.

CKD patients who are not prescribed dialysis are typically conservatively managed. The current standard of care in treating anemia associated with CKD includes erythropoiesis stimulating agents (ESA), iron supplementation, and RBC blood transfusions. ESAs are effective and have generally shown few adverse side effects. Use of ESAs maintains target Hb levels in the majority of patients and reduce the need for blood transfusions. FDA-approved ESAs include recombinant glycoproteins, such as Epoetin alfa (Epogen/Procrit), Darbepoetin alfa (Aranesp), Methoxy polyethylene glycol-epoetin beta (Mircera), Epoetin alfa-epbx (Retacrit); and biosimilars to Epogen/Procrit. ESAs are typically administered intravenously; they may however be administered subcutaneously. Iron supplementation can be in the form of intravenous (IV) iron or oral iron. In some embodiments, methods provided herein involve the use of hepcidin antagonists (e.g., hemojuvelin antagonists) in combination with erythropoiesis stimulating agents (ESA), iron supplementation, and/or RBC blood transfusions.

In some embodiments, CKD patients who have not been prescribed dialysis are often not in the habit of making frequent hospital visits. In some embodiments, because ESAs are often administered intravenously, prescription of ESAs for patients having a CKD that is non-dialysis dependent (CKD-NDD) often presents a significant burden in that patients must make frequent hospital visits to receive the ESA. Furthermore, patients have reported experiencing pain during subcutaneous administration of ESAs. Additionally, recent concerns about the safety of ESAs, specifically higher cardiovascular risk, cancer progression and increased mortality have decreased the usage of ESAs worldwide. And as with HIF-PHI drugs, a major efficacy concern of ESAs is an overcorrection of hemoglobin levels in serum, which can lead to adverse cardiovascular outcomes.

There are several physiological factors and diagnostic criteria that influence a physician's decision to prescribe dialysis. Exemplary physiological factors include GFR (and in turn the stage of CKD), ACR, calcium levels, and BMI. Patients having GFR values above 15 ml/min are not candidates for dialysis. In contrast, for patients having GFR values below 15 ml/min, dialysis is recommended. Patients having GFR levels below 5 or 6 must be put on dialysis immediately. See Tattersal et al., Nephrol Dial Transplant (2011) 26:2082-2086, which is herein incorporated by reference. When the GFR is above 15, other markers such as ACR, serum calcium levels, BMI, and presence of uremic symptoms such as nausea, anorexia, insomnia, and fatigue, are considered in the decision to recommend dialysis. Dialysis may not be recommended when ACR is below 300. Conversely, dialysis is typically recommended in patients that present with uremic symptoms such as nausea and anorexia. See Wang et al., Renal Failure 2021 43 (31): 216-222, which is herein incorporated by reference.

Additional, non-physiological factors influence the physician's decision to recommend dialysis. Prognosis, anticipated quality of life (with or without dialysis), treatment burden (if dialysis is undertaken), support by family members (including assistance in traveling to and from inpatient or outpatient facilities), and patient preferences, all play a role in the decision. A decision against dialysis is, usually made jointly between physician and patient, according to their preferences and in the light of best evaluation of these factors and the physiological factors above. See Murtagh et al., Nephrol Dial Transplant (2007) 22:1955-1962, which is herein incorporated by reference.

There are two types of dialysis: hemodialysis and peritoneal dialysis. In peritoneal dialysis, a cleansing fluid flows through a tube (catheter) into part of the abdomen. The lining of the abdomen (peritoneum) acts as a filter and removes waste products from the blood. After a set period of time, the fluid with the filtered waste products flows out of the abdomen and is discarded. Peritoneal dialysis can be administered in outpatient facilities of otherwise in the home by a qualified professional. In hemodialysis, blood is removed from the body, filtered through an artificial kidney machine, and then returned to the body. Hemodialysis is typically done in hospital. CKD patients on dialysis who suffer from anemia of CKD typically receive high doses of IV EPO and IV iron supplementation.

In general, about 15 percent of anemic CKD patients who are not on dialysis are treated with oral iron supplementation. (This iron supplementation treatment may be suspended by the physician if dialysis is ordered.) Oral iron may be administered at home or in an outpatient facility. For these patients, IV treatments (e.g., intravenous antibody) treatments are burdensome because outpatient facilities are often not equipped for intravenous antibody delivery.

The present disclosure provides methods of treating anemia of kidney disease with a hepcidin antagonist, such as a hemojuvelin antagonist (e.g., an anti-hemojuvelin antibody). The present disclosure provides anti-hemojuvelin antibodies that have a demonstrated safety profile. For instance, in some embodiments, methods are provided that involve administration of any of the disclosed antibodies to a subject and that do not cause an overcorrection of hemoglobin levels in serum of the subject. In some embodiments, these antibodies may be formulated for subcutaneous delivery. Further, in some embodiments, the present disclosure is directed to methods for subcutaneous delivery of an anti-hemojuvelin antibody to treat anemia of CKD. The present disclosure is further directed to methods for subcutaneous delivery of a hemojuvelin antagonist (e.g., an anti-hemojuvelin antibody) in the home or an outpatient facility or physician's office, or the like, to treat anemia of CKD. The presently disclosed methods of subcutaneous administration of a hemojuvelin antagonist may be particularly suitable for CKD patients not on dialysis because subcutaneous administration may be performed in the home.

In some embodiments, the presently disclosed methods may be particularly suitable for subjects that are not on dialysis, such as CKD patients not on dialysis. These methods may be suitable for CKD-NDD patients. These methods may be suitable for CKD patients on dialysis. These methods may be particularly suitable for the population of anemic CKD patients who are not on dialysis and are candidates for treatment with oral iron supplementation.

The presently disclosed methods may be particularly suitable for treating anemia in CKD patients not on dialysis who are identified as lacking a response (non-responsive) to oral iron (or having a CKD that is refractory to oral iron). The presently disclosed methods may be particularly suitable for treating anemia in CKD patients not on dialysis who are identified as non-responsive to IV iron. These methods may be particularly suitable for treating anemia in CKD patients who have not undergone a nephrectomy (kidney removal). These methods may be particularly suitable for treating anemia in CKD patients who are not candidates for IV iron (having TSAT of less than 20% and serum iron levels of less than 20 ng/ml).

These methods may be particularly suitable for CKD patients who i) have elevated hepcidin levels, ii) reduced endogenous EPO levels, iii) functional iron deficiency, iv) nutritional iron deficiency (deficient uptake of iron from diet), and/or v) reduced hemoglobin levels. In CKD, when kidneys begin failing, hepcidin levels rise, and a concomitant nutritional iron deficiency and EPO resistance is observed. Thus, the disclosure provides methods of administration of any of the disclosed hemojuvelin antagonists (e.g., anti-hemojuvelin antibodies) to a CKD patient not on dialysis who exhibits low iron levels or low TSAT levels. The disclosed antibodies may be administered to patients where any one of the following is observed: a) elevated hepcidin levels than normal (non-anemic) subjects; b) a nutritional iron deficiency (less than 100 ng/ml serum iron); or c) EPO resistance.

In some embodiments, the disclosed methods are directed to treating anemia in CKD patients who exhibit a GFR above 15 ml/min. In some embodiments, the disclosed methods are directed to treating anemia in CKD patients who exhibit a GFR above 15 ml/min but below 90 ml/min.

In some embodiments of the disclosed methods, the subject to which the hemojuvelin antagonist is administered is identified as having a level of GFR of in the range of 15 to less than 90 mL/min per 1.73 m². The subject may be identified as having a level of GFR of in the range of 15 to less than 60 mL/min per 1.73 m². The subject may have a level of GFR of in the range of 15 to less than 30 mL/min per 1.73 m². The subject may have a level of GFR of in the range of less than 30 mL/min per 1.73 m², less than 15 mL/min per 1.73 m², or less than 7 mL/min per 1.73 m².

Accordingly, provided herein are methods of treating subjects suffering from CKD at a stage between stages 1 and 5, as provided in Table 13, below. In some embodiments, the chronic kidney disease is classified as being at a stage in the range of stages 1 to 4. In some embodiments, the CKD is classified as being at a stage in the range of stages 2 to 4. In particular embodiments, the CKD is classified as being at stage 4.

In some embodiments, the disclosed methods are directed to treating anemia in CKD patients who exhibit albuminuria. Albuminuria (or proteinuria) refers to the presence of albumin in the urine and is determined based on the albumin-to-creatinine (ACR) ratio. An ACR of less than 30 is categorized as A1 albuminuria, while an ACR ranging from 30-300 is categorized as A2, and an ACR above 300 is categorized as A3. In some embodiments, the disclosed methods are directed to treating anemia in CKD patients who exhibit a ACR below 300. In some embodiments, the disclosed methods are directed to treating anemia in CKD patients who exhibit a ACR between 30 and 300.

In some embodiments, the disclosed methods are directed to treating anemia in CKD patients who exhibit reduced levels of endogenous EPO. In some embodiments, the disclosed methods are directed to treating anemia in CKD patients who exhibit functional iron deficiency, or nutritional iron deficiency. In some embodiments, the disclosed methods are directed to treating anemia in CKD patients who exhibit shortened red blood cell survival. Subjects suffering from anemia associated with CKD typically present with reduced endogenous EPO levels and/or functional iron deficiency. In some embodiments, the disclosed methods are directed to treating anemia in CKD patients who exhibit reduced hepcidin levels. In some embodiments, the disclosed methods are directed to treating anemia in CKD patients who exhibit non-responsiveness to oral iron or IV iron (such as MonoFerric®).

Further aspects of the disclosure, including a description of defined terms, are provided below.

### I. Definitions

Administering: As used herein, the terms “administering” or “administration” means to provide a complex to a subject in a manner that is physiologically and/or pharmacologically useful (e.g., to treat a condition in the subject).

Anemia of Chronic Disease: As used herein, the term “anemia of chronic disease” (ACD) refers to a haematological disorder arising in the context of an illness or condition that elicits an active immune/inflammatory response resulting in a deficiency in the ability of blood to transport oxygen. Chronic conditions (e.g., lasting 3 months or longer) can give rise to a low level of iron in the blood, despite normal or even increased levels of iron stores in macrophages and hepatocytes. In this context, inflammation may prevent the use of stored iron to product sufficient healthy red blood cells, leading to anemia. In some embodiments, ACD is the result of a deficiency in red blood cells, a deficiency in hemoglobin, and/or a deficiency in total blood volume. In some embodiments, ACD is associated with an alteration of iron metabolism and diversion of body iron (e.g., via macrophage sequestration), haemophagocytosis, reduction in erythropoiesis, and/or diminished response to erythropoietin stimulation. In some embodiments, ACD may be associated with or characterized by one or more of the following: impaired production of erythropoietin (EPO), blunted marrow erythroid response to EPO, iron-restricted erythropoiesis, and a diminished pool of EPO-responsive cells in combination with an associated chronic condition associated with inflammation. Accordingly, in some embodiments, low serum iron levels can provide diagnostic indicia of the presence of ACD in a subject when observed in the presence of an underlying chronic condition or disease. In some embodiments, ACD is an iron-restricted anemia, which may be characterized by a functional iron deficiency, which may present in a subject as a result of iron accumulation in tissue macrophages. Conditions associated with ACD include diseases which share features of immune activation. Examples of conditions associated with ACD include, without limitation, chronic kidney disease and renal disease (e.g., chronic renal failure).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′) 2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (A), epsilon (€), gamma (γ) or mu (u) heavy chain. In some embodiments, the heavy chain of an antibody described herein can comprise a human alpha (a), delta (A), epsilon (€), gamma (γ) or mu (u) heavy chain. In a particular embodiment, an antibody described herein comprises a human gamma 1 CH1, CH2, and/or CH3 domain. In some embodiments, the amino acid sequence of the V_{H} domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the V_{H} domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Affinity Matured Antibody: “Affinity Matured Antibody” is used herein to refer to an antibody with one or more alterations in one or more CDRs, which result in an improvement in the affinity (i.e. KD, kd or ka) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Chronic kidney disease (CKD) is defined by abnormalities of kidney structure or function that present in the patient for more than 3 months. In the US alone, approximately 5.6 million patients have a glomerular filtration rate (GFR) below 45 ml/min. A GFR of 60 mL/min/1.73 m² or higher is in the normal range. The standard way to estimate GFR is an assay of creatinine levels in a blood sample. Creatinine is a waste product from the digestion of dietary protein and normal breakdown of muscle tissue. A substantially reduced GFR over a duration of more than a week suggests some kidney damage. A substantially reduced GFR for more than 3 months is usually diagnosed as CKD. GFR is expressed in units of ml/min. Adjusted for body surface area, GFR is expressed in units of ml/min/1.73 m². Estimated GFR (eGFR) is recommended by clinical practice guidelines for routine evaluation of GFR, whereas measured GFR is recommended as a confirmatory test when a more accurate assessment is required.

CKD comprises a group of pathologies that affect kidney function resulting from damage to renal structures. There are at least three classes of criteria by which doctors identify a patient as having CKD. The first is identifying the cause of CKD; the second is assigning a GFR category; and the third is assigning an albuminuria category. Determining the cause of CKD is based on identifying which renal structure is damaged and is important to establishing whether the patient has a systemic or localized condition. There are three causes of CKD: i) glomerular, ii) tubulointerstitial and iii) vascular disease. Classification into the categories of glomerular, tubulointerstitial and vascular is based on analysis of pathological-anatomic findings from kidney biopsy or imaging. Nephron loss or fibrosis affecting glomeruli or tubules is classified as either glomerular or tubulointerstitial disease. Lastly, findings from kidney biopsy or imaging revealing alterations to renal vasculature are classified as vascular disease.

There are 5 stages of CKD: G1, G2, G3, G4 and G5 (also referred to as stage 1, stage 2, stage 3, stage 4, and stage 5). The G3 stage can be broken further into sub-stages 3a and 3b. The GFR rates that correspond to each stage are provided in Table 13, below:

TABLE 13

| CDK | | GFR (mL/min |
|---|---|---|
| Stage | Description | per 1.73 m²) |
| 1 | Normal kidney function but evidence of disease | ≥90 |
| 2 | Mildly reduced kidney function | 60-89 |
| 3a | Reduced kidney function | 45-59 |
| 3b | Reduced kidney function | 30-44 |
| 4 | Severely reduced kidney function | 15-29 |
| 5 | Severely reduced kidney function; dialysis | <15 |

### Based on KDOQI Disease Definition

See National Kidney Foundation, K/DOQI clinical practice guidelines for chronic kidney disease: Evaluation, classification, and stratification, Am. J. Kidney Dis. 2002:39 (Suppl 1): S1-S266, which is herein incorporated by reference.

CKD-NDD: CKD patients who are not prescribed dialysis may be classified as being “non-dialysis dependent”, or CKD-NDD (or NDD-CKD). These patients are not undergoing dialysis (or dialytic) therapy.

Comorbidity: As used herein, a “comorbidity” refers to one or more conditions or disorders that co-occur with (or are coincident with) a primary condition (such as CKD) in an individual.

Albuminuria (or proteinuria) refers to the presence of albumin in the urine. The albuminuria category is determined based on the albumin-to-creatinine (ACR) ratio. An ACR of less than 30 is categorized as A1 albuminuria, while an ACR ranging from 30-300 is categorized as A2, and an ACR above 300 is categorized as A3.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The V_{H} and VL regions can be further subdivided into regions of hypervariability, also known as “complementarity determining regions” (“CDR”), interspersed with regions that are more conserved, which are known as “framework regions” (“FR”). Each V_{H} and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. Sec, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® (imgt.org), Lefranc, M. P. et al., Nucleic Acids Res., 27:209-212 (1999); Ruiz, M. et al., Nucleic Acids Res., 28:219-221 (2000); Lefranc, M. P., Nucleic Acids Res., 29:207-209 (2001); Lefranc, M. P., Nucleic Acids Res., 31:307-310 (2003); Lefranc, M. P. et al., In Silico Biol., 5, 0006 (2004) [Epub], 5:45-60 (2005); Lefranc, M. P. et al., Nucleic Acids Res., 33: D593-597 (2005); Lefranc, M. P. et al., Nucleic Acids Res., 37: D1006-1012 (2009); Lefranc, M. P. et al., Nucleic Acids Res., 43: D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). Ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

Generally, there are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although exemplary embodiments use Kabat or Chothia defined CDRs.

CDR-grafted antibody: The term “CDR-grafted antibody” refers to antibodies which comprise heavy and light chain variable region sequences from one species but in which the sequences of one or more of the CDR regions of V_{H} and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (c) S, T; (f) Q, N; and (g) E, D.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human hemojuvelin and non-human primate hemojuvelin) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Effective Amount: As used herein, “an effective amount” refers to the amount of each active agent (e.g., hepcidin antagonist, anti-HJV antibody) required to confer therapeutic effect on the subject (such as in treating anemia associated with CKD, or anemia in a subject having a certain level of GFR), cither alone or in combination with one or more other active agents. In some embodiments, the therapeutic effect is reduced hepcidin level or activity, increased level of transferrin saturation (TSAT %), decreased level of circulating transferrin level, and/or alleviated disease conditions (e.g., reduced anemia).

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Glomerular filtration rate: As used herein, “glomerular filtration rate” (GFR) is a measure of kidney function that describes the flow rate of filtered fluid through the kidney. GFR is a measurement of the volume of blood that passes through the glomeruli each minute (glomeruli are clusters of capillaries in the nephron that filter waste from the blood). The standard for calculating a measured GFR is directly measuring the plasma or urinary clearance of an exogenous filtration marker (e.g., inulin or iohexol) in the subject. In other embodiments, an exemplary method for calculating estimated GFR (eGFR) in patients is the mean of urea and creatinine clearance (CC), as calculated from a 24-hour urine collection and normalized to a human body surface area of 1.73 m². Sec Tattersal et al., Nephrol Dial Transplant (2011) 26:2082-2086, herein incorporated by reference. In some embodiments, “glomerular filtration rate” refers to estimated GFR (eGFR). In some embodiments, “glomerular filtration rate” refers to measured GFR.

Hemojuvelin (HJV): As used herein, the term “hemojuvelin (HJV)” (also known as repulsive guidance molecule C (RGMc) or hemochromatosis type 2 protein (HFE2)) refers to a membrane-bound and soluble form protein that regulates hepcidin production through the BMP/SMAD signaling pathway. The HFE2 gene encodes two known classes of GPI-anchored and glycosylated HJV molecules, which are targeted to the membrane and undergo distinct fates. HJV exists in multiple isoforms, including two soluble isoforms and two membrane-associated isoforms. In some embodiments, a predominant membrane-associated isoform is a disulfide-linked two-chain form composed of N- and C-terminal fragments. In some embodiments, a full-length single-chain isoform associates with the membrane, but is released from the cell surface and accumulates in extracellular fluid. In some embodiments, HJV may be of human (NCBI Gene ID 148738), non-human primate (e.g., NCBI Gene ID 698805), or rodent (e.g., NCBI Gene ID 69585 or NCBI Gene ID 310681) origin. In addition to HJV (RGMc), the repulsive guidance molecule family includes repulsive guidance molecule A (RGMa) and repulsive guidance molecule B (RGMb). RGMa and RGMb are expressed in the central nervous system during development and are thought to be involved in controlling axonal patterning and neuronal survival, while HJV is produced in the liver and in cardiac and skeletal muscle.

Hepcidin: As used herein, a “hepcidin” refers to an iron-regulating peptide hormone primarily made in the liver that is encoded by the HAMP gene. In some embodiments, hepcidin controls the delivery of iron to blood plasma from intestinal cells absorbing iron, from erythrocyte-recycling macrophages, and from iron-storing hepatocytes. In some embodiments, hepcidin inhibits iron transport by binding to the iron export channel ferroportin which is located on the basolateral surface of gut enterocytes and the plasma membrane of reticuloendothelial cells (macrophages). In some embodiments, inhibiting ferroportin prevents iron from being exported and the iron is sequestered in the cells. In some embodiments, by inhibiting ferroportin, hepcidin prevents enterocytes from allowing iron into the hepatic portal system, thereby reducing dietary iron absorption. Hepcidin expression involves multiple aspects, including, for example, transcription of the HAMP gene, translation of the transcribed mRNA, and the posttranslational processing of the hepcidin precursor into the bioactive hepcidin-25 peptide

(DTHFPICIFCCGCCHRSKCGMCCKT (SEQ ID NO: 129)). In some embodiments, hepcidin expression is modulated via the hemojuvelin-induced BMP signaling pathway. In some embodiments, hepcidin expression is modulated via the IL-6-JAK-STAT signaling pathway.

Hepcidin Antagonist: As used herein, a “hepcidin antagonist” refers to an agent that reduces hepcidin expression and/or hepcidin activity (directly or indirectly). In some embodiments, a hepcidin antagonist inhibits hepcidin-induced ferroportin degradation. Accordingly, in some embodiments, a hepcidin antagonist targets hepcidin function indirectly through the hepcidin stimulatory pathway to decrease hepcidin expression. In some embodiments, a hepcidin antagonist targets hepcidin function directly, e.g., by binding the hepcidin peptide to sequester free hepcidin or by binding ferroportin to inhibit the hepcidin-ferroportin binding interaction, thereby decreasing hepcidin-induced ferroportin degradation. In some embodiments, a hepcidin antagonist is a ferroportin inhibitor that disrupts ferroportin-hepcidin interactions, such as, for example, as disclosed in Ross S L, et al., Identification of Antibody and Small Molecule Antagonists of Ferroportin-Hepcidin Interaction. Front Pharmacol. 2017 Nov. 21; 8:838; Fung E., et al., High-Throughput Screening of Small Molecules Identifies Hepcidin Antagonists. Molecular Pharmacology March 2013, 83 (3) 681-690; and Angeliki Katsarou and Kostas Pantopoulos, Hepcidin Therapeutics. Pharmaceuticals (Basel). 2018 December; 11 (4): 127, the relevant contents of each of which are incorporated herein by reference.

Hemojuvelin antagonist: As used herein, the term “hemojuvelin antagonist,” refers to a molecule that reduces expression of hemojuvelin or inhibits hemojuvelin, e.g., by binding to hemojuvelin. In some embodiments, the hemojuvelin antagonist is an antisense oligonucleotide (sec. e.g., U.S. Pat. No. 7,534,764; U.S. Patent Publication No. US 2014/127325; and International Publication No. WO 2016/180784, which are incorporated herein by reference). In some embodiments, the hemojuvelin antagonist is an antibody. In other embodiments, the hemojuvelin antagonist is a small molecule compound that inhibits hemojuvelin, e.g., by competitive binding and/or chemical modification of hemojuvelin.

HJV-induced BMP signaling: As used herein, the term “HJV-induced BMP signaling” refers to signaling through BMP receptors that is induced by Hemojuvelin (HJV), which is a membrane bound co-receptor for bone morphogenetic protein (BMP) signaling. As discussed in Xia Y, et al., Hemojuvelin regulates hepcidin expression via a selective subset of BMP ligands and receptors independently of neogenin, Blood. 2008 May 15; 111 (10): 5195-5204, in hepatocytes, HJV-induced BMP signaling positively regulates hepcidin mRNA expression. In some embodiments, HJV binds to BMP2, BMP4, BMP5, or BMP6 to induce BMP signaling, e.g., to positively regulate hepcidin levels in hepatocytes. In some embodiments, cleavage of HJV by matriptase-2 reduces the amount of cell surface HJV available to participate in BMP signaling. In some embodiments, induction of BMP signaling by HJV is independent of neogenin. However, in some embodiments, neogenin facilitates induction of BMP signaling by HJV, as discussed in Zhao et al, Neogenin Facilitates the Induction of Hepcidin Expression by Hemojuvelin in the Liver, J Biol Chem. 2016 Jun. 3; 291(23): 12322-12335. In some embodiments, BMP6 is responsible for iron-dependent activation of the Smad signaling. In some embodiments, BMP6 is secreted from liver sinusoidal endothelial cells and binds to a BMP receptor (BMPR) on hepatocytes and thereby activates the SMAD signaling cascade. In such embodiments, HJV serves as a co-receptor for such BMP6, e.g., to positively regulate hepcidin levels in hepatocytes. In some embodiments, BMPs transduce signals by binding to one or a combination of type I and II serine/threonine kinase receptors. BMP type II receptors include BMPRII, ActRIIA, and ActRIIB. BMP type I receptors include ALK3, ALK6, and ALK2. In some embodiments, upon ligand binding, constitutively active type II receptors phosphorylate type I receptors, and type I receptors then phosphorylate intracellular receptor-activated Smads (R-Smads), namely Smad 1, Smad 5 and/or Smad 8. In such embodiments, activated R-Smads complex with the common partner Smad4 and translocate to the nucleus to regulate gene transcription, e.g., induction of hepcidin expression.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences (e.g., CDRs grafted in a heterologous framework).

Humanized antibody: The term “humanized antibody” refers to antibodies which comprise heavy and light chain variable region sequences from a non-human species (e.g., a mouse) but in which at least a portion of the V_{H} and/or V_{L} sequence has been altered to be more “human-like”, i.e., more similar to human germline variable sequences. One type of humanized antibody is a CDR-grafted antibody, in which human CDR sequences are introduced into non-human V_{H} and V_{L} sequences to replace the corresponding nonhuman CDR sequences. In one embodiment, humanized anti-hemojuvelin antibodies and antigen binding portions are provided. Such antibodies may be generated by obtaining murine anti-hemojuvelin monoclonal antibodies using traditional hybridoma technology followed by humanization using in vitro genetic engineering, such as those disclosed in Kasaian et al PCT publication No. WO 2005/123126 A2.

Isolated antibody: An “isolated antibody”, as used herein, is intended to refer to an antibody that is substantially free of other antibodies having different antigenic specificities (e.g., an isolated antibody that specifically binds hemojuvelin is substantially free of antibodies that specifically bind antigens other than hemojuvelin). An isolated antibody that specifically binds hemojuvelin may, however, have cross-reactivity to other antigens, such as other repulsive guidance molecule (RGM) proteins (e.g., RGMa and/or RGMb). Moreover, an isolated antibody may be substantially free of other cellular material and/or chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Kidney damage: As used herein, the term “kidney damage” refers to structural or functional abnormalities of the kidney, with or without a decreased estimated or measured glomerular filtration rate (GFR), that manifests as pathological abnormalities or markers of kidney disease, including abnormalities in the composition of blood or urine or abnormalities in imaging tests.

Anemia associated with kidney damage, as used herein, is anemia that is coincident with, or directly or indirectly caused by, kidney damage.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

Nephrectomy: As used herein, the term “nephrectomy” refers to the removal of part or all of one or more kidneys.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. One embodiment of the disclosure provides fully human antibodies capable of binding human hemojuvelin which can be generated using techniques well known in the art, such as, but not limited to, using human Ig phage libraries such as those disclosed in Jermutus et al., PCT publication No. WO 2005/007699 A2.

Selective: As used herein, the term “selective” or “selectively” refers to the ability of a molecule to produce an effect in relation to its target molecule compared to a reference molecule. For example, a molecule that selectively inhibits its target molecule means that this molecule is capable of inhibiting its target molecule with a degree that is distinguishable from a reference molecule in an inhibition assay or other inhibitory context. For example, with respect to an inhibitor, the term, “selectively inhibits”, refers to the ability of the inhibitor to inhibit its target molecule with a degree that is distinguishable from a reference molecule that is not substantially inhibited in an inhibition assay, e.g., to an extent that permit selective inhibition of the target molecule, as described herein. For example, the half maximal inhibitory concentration (IC50) for the target molecule and/or the reference molecule can be tested in a kinase potency assay as described in Asshoff, M. et al., Momelotinib inhibits ACVR1/ALK2, decreases hepcidin production, and ameliorates anemia of chronic disease in rodents. Blood. 2017 Mar. 30; 129(13): 1823-1830 (e.g., Kinase potency assay by Carna Biosciences). In this assay, inhibitor solution (e.g., solution containing the selective inhibitor to be tested)/kinase substrate is mixed with target molecule solution (e.g., ALK2) or reference molecule solution (e.g., JAK1 or JAK2), and incubated under room temperature for 1 hour. Once the reaction is terminated, the signal produced by enzymatic activity on the substrate can be measured. The half maximal inhibitor concentration for the target molecule and the reference molecule can be calculated. In some embodiments, a molecule described herein selectively binds to a target molecule. In some embodiments, a molecule described herein selectively inhibits to a target molecule. In some embodiments, a molecule described herein selectively antagonizes to a target molecule. In some embodiments, a molecule described herein selectively neutralizes to a target molecule.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M. 10⁻⁷ M, 10⁻⁸ M, 10⁻⁹ M, 10⁻¹⁰ M, 10⁻¹¹ M. 10⁻¹² M, 10⁻¹³ M, or less. In some embodiments, an antibody specifically binds to hemojuvelin.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having anemia associated with kidney disease and/or one or more conditions which are associated with, or may give rise to, anemia associated with kidney disease and/or a functional iron deficiency.

Treatment: As used herein, the term “treating” or “treatment” refers to the application or administration of a composition including one or more active agents to a subject, who has a target disease or disorder (such as anemia of CKD), a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying or preventing the development or progression of the disease, or reducing disease severity.

### II. Anti-Hemojuvelin (HJV) Antibodies

In some embodiments, the hemojuvelin antagonist binds to one or more proteins of the repulsive guidance molecule (RGM) family, including RGMa, RGMb, and RGMc (HJV). In some embodiments, the hemojuvelin antagonist selectively binds hemojuvelin (RGMc) over RGMa and RGMb. In some embodiments, the hemojuvelin antagonist is an antisense oligonucleotide that reduces expression of hemojuvelin (see, e.g., U.S. Pat. No. 7,534,764; U.S. Patent Publication No. US 2014/127325; and International Publication No. WO 2016/180784, which are incorporated herein by reference). In some embodiments, the hemojuvelin antagonist is a small molecule compound that inhibits hemojuvelin, e.g., by competitive binding and/or chemical modification of hemojuvelin.

In some embodiments, the hemojuvelin antagonist is an antibody (e.g., HA001-HA012) specific for hemojuvelin and/or one or more proteins of the RGM protein family (e.g., RGMa, RGMb). Appropriate antibodies specific for hemojuvelin and/or one or more RGM proteins that may be useful in certain methods provided herein are provided for example, in U.S. Pat. Nos. 10,118,958; and 8,507,435; U.S. Patent Publication Nos. US 2013/330343; US 2015/166672; and US 2017/029499; and International Publication Nos. WO 2015/171691; and WO 2018/009624, which are incorporated herein by reference.

Provided herein, in some aspects, are antibodies that bind to human hemojuvelin (HJV) with high specificity and affinity. In some embodiments, the anti-HJV antibody described herein specifically binds to any extracellular epitope of a HJV or an epitope that becomes exposed to an antibody. In some embodiments, anti-HJV antibodies provided herein bind specifically to HJV from human, non-human primates, mouse, rat, etc. In some embodiments, anti-HJV antibodies provided herein bind to human HJV. In some embodiments, the anti-HJV antibody described herein binds to an amino acid segment of a human or non-human primate HJV.

In some embodiments, the anti-HJV antibody described herein specifically binds to an epitope on human HJV. Human HJV is a 426 amino acid protein with a predicted N-terminal signal peptide of 31 amino acids and a C-terminal GPI-attachment signal of 45 amino acids. An exemplary human HJV amino acid sequence is set forth in SEQ ID NO: 128:

(SEQ ID NO: 128)
MGEPGQSPSPRSSHGSPPTLSTLTLLLLLCGHAHSQCKILRCNAEYVSST
LSLRGGGSSGALRGGGGGGRGGGVGSGGLCRALRSYALCTRRTARTCRGD
LAFHSAVHGIEDLMIQHNCSRQGPTAPPPPRGPALPGAGSGLPAPDPCDY
EGRFSRLHGRPPGFLHCASFGDPHVRSFHHHFHTCRVQGAWPLLDNDFLF
VQATSSPMALGANATATRKLTIIFKNMQECIDQKVYQAEVDNLPVAFEDG
SINGGDRPGGSSLSIQTANPGNHVEIQAAYIGTTIIIRQTAGQLSFSIKV
AEDVAMAFSAEQDLQLCVGGCPPSQRLSRSERNRRGAITIDTARRLCKEG
LPVEDAYFHSCVFDVLISGDPNFTVAAQAALEDARAFLPDLEKLHLFPSD
AGVPLSSATLLAPLLSGLFVLWLCIQ

In some embodiments, the anti-HJV antibody described herein may bind to a fragment of a human HJV. The fragment of HJV may be between about 5 and about 425 amino acids, between about 10 and about 400 amino acids, between about 50 and about 350 amino acids, between about 100 and about 300 amino acids, between about 150 and about 250 amino acids, between about 200 and about 300 amino acids, or between about 75 and about 150 amino acids in length. The fragment may comprise a contiguous number of amino acids from RGMc. An exemplary amino acid of a HJV fragment is set forth in SEQ ID NO: 123:

(SEQ ID NO: 123)
QCKILRCNAEYVSSTLSLRGGGSSGALRGGGGGGRGGGVGSGGLCRALRS
YALCTRRTARTCRGDLAFHSAVHGIEDLMIQHNCSRQGPTAPPPPRGPAL
PGAGSGLPAPDPCDYEGRFSRLHGRPPGFLHCASFGDPHVRSFHHHFHTC
RVQGAWPLLDNDFLFVQATSSPMALGANATATRKLTIIFKNMQECIDQKV
YQAEVDNLPVAFEDGSINGGDRPGGSSLSIQTANPGNHVEIQAAYIGTTI
IIRQTAGQLSFSIKVAEDVAMAFSAEQDLQLCVGGCPPSQRLSRSERNRR
GAITIDTARRLCKEGLPVEDAYFHSCVFDVLISGDPNFTVAAQAALEDAR
AFLPDLEKLHLFPSD

In some embodiments, the anti-HJV antibody described herein binds to different epitopes within a human HJV or a human HJV fragment.

In some embodiments, the anti-HJV antibody interacts with an epitope within amino acids 160-190 of SEQ ID NO: 123. In some embodiments, the anti-HJV antibody interacts with an epitope having an amino acid sequence of amino acids 170-183 of SEQ ID NO: 123. In some embodiments, the anti-HIV antibody interacts with an epitope having the amino acid sequence of SSPMALGANATATR (SEQ ID NO: 121). In some embodiments, the anti-HJV antibody interacts with different segments within SSPMALGANATATR (SEQ ID NO: 121). In some embodiments, the anti-HJV antibody interacts with amino acids 170-171, amino acids 171-180, amino acids 180-182, and amino acids 182-183 of SEQ ID NO: 123. In some embodiments, the antibody interacts with amino acids 170 (S), 171 (S), 180 (T), 182 (T) and 183 I of SEQ ID NO: 123. In some embodiments, hHA-008 interacts with the epitope SSPMALGANATATR (SEQ ID NO: 121). In some embodiments, hHA-008 interacts with amino acids 170 (S), 171 (S), 180 (T), 182 (T) and 183 (R) of SEQ ID NO: 123.

In some embodiments, the anti-HJV antibody interacts with an epitope within amino acids 160-190 of SEQ ID NO: 123 and/or amino acids 280-310 of SEQ ID NO: 123. In some embodiments, the anti-HIV antibody interacts with an epitope within amino acids 169-182 of SEQ ID NO: 123 and/or amino acids 289-300 of SEQ ID NO: 123. In some embodiments, the anti-HJV antibody interacts with an epitope within amino acids 169-182 of SEQ ID NO: 123 and amino acids 289-300 of SEQ ID NO: 123. In some embodiments, the anti-HJV antibody interacts with an epitope having the amino acid sequence of TSSPMALGANATAT (SEQ ID NO: 122) and amino acid sequence SQRLSRSERNRR (SEQ ID NO: 127). In some embodiments, the anti-HJV antibody interacts with different segments within TSSPMALGANATAT (SEQ ID NO: 122) and SQRLSRSERNRR (SEQ ID NO: 127). In some embodiments, the anti-HJV antibody interacts with amino acids 169-171, amino acids 171-180, and amino acids 180-182 of SEQ ID NO: 123, and amino acids 289-293, amino acids 293-294, amino acids 294-295, amino acids 295-297 and amino acids 297-300 of SEQ ID NO: 123. In some embodiments, the antibody interacts with amino acids 169 (T), 170 (S), 171 (S), 180 (T), 182 (T), 289 (S), 293 (S), 294 (R), 295 (S), 297 (R), and 300 (R) of SEQ ID NO: 123. In some embodiments, hHA-008-QL interacts with different segments within TSSPMALGANATAT (SEQ ID NO: 122) and SQRLSRSERNRR (SEQ ID NO: 127). In some embodiments, hHA-008-QL interacts with amino acids 169 (T), 170 (S), 171 (S), 180 (T), 182 (T), 289 (S), 293 (S), 294 (R), 295 (S), 297 (R), and 300 (R) of SEQ ID NO: 123.

In some embodiments, the anti-HJV antibodies described herein are affinity matured clones. In some embodiments, an anti-HJV antibody specifically binds a HJV (e.g., a human or non-human primate HJV) with binding affinity (e.g., as indicated by K_{D}) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. For example, the anti-HJV antibodies of the present disclosure can bind to a hemojuvelin protein (e.g., human hemojuvelin) with an affinity between 5 pM and 500 nM, e.g., between 50 pM and 100 nM, e.g., between 500 pM and 50 nM. The disclosure also includes antibodies that compete with any of the antibodies described herein for binding to a hemojuvelin protein (e.g., human hemojuvelin) and that have an affinity of 100 nM or lower (e.g., 80 nM or lower, 50 nM or lower, 20 nM or lower, 10 nM or lower, 500 pM or lower, 50 pM or lower, or 5 pM or lower). The affinity and binding kinetics of the anti-HJV antibody can be tested using any suitable method including but not limited to biosensor technology (e.g., OCTET or BIACORE). In some embodiments, the anti-HJV antibodies described herein binds to HJV with a K_{D} of sub-nanomolar range. In some embodiments, the anti-HJV antibodies described herein selectively binds to RGMc, but not RGMa or RGMb.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH7.4, 150 mM NaCl, 0.005% (v/v) surfactant P20) and PBS buffer (10 mM PO4-3, 137 mM NaCl, and 2.7 mM KCl). These techniques can be used to measure the concentration of bound proteins as a function of target protein concentration. The concentration of bound protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The heavy chain (HC) and light chain (LC) sequences, heavy chain variable domain (VH) and light chain variable domain (VL), CDR sequences, and heavy chain and light chain constant region sequences of non-limiting examples of anti-HJV antibodies are provided in Table 1.

TABLE 1
Examples of anti-HJV antibodies (CDRs according to the Kabat definition)

| humanized Hemojuvelin | | | SEQ |
|---|---|---|---|
| Antagonist (HA) Antibody | | Sequences | ID NO |
| hHA-001 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLETSDGDTFLE | 4 |
| | LC CDR2 | EVSTRFS | 5 |
| | LC CDR3 | FQVTHDPMT | 6 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLETSDGDTFLEW | 8 |
| | | FQQRPGQSPRLLIYEVSTRFSGVPDRFSGSGSGTDFTLKI | |
| | | SRVEAEDVGVYYCFQVTHDPMTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSFNRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLETSDGDTFLEW | 52 |
| | | FQQRPGQSPRLLIYEVSTRFSGVPDRFSGSGSGTDFTLKI | |
| | | SRVEAEDVGVYYCFQVTHDPMTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-002 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLETSDGDTFLE | 4 |
| | LC CDR2 | EVSSRES | 49 |
| | LC CDR3 | MQVTHDPLT | 24 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLETSDGDTFLEW | 30 |
| | | FQQRPGQSPRLLIYEVSSRFSGVPDRFSGSGSGTDFTLKI | |
| | | SRVEAEDVGVYYCMQVTHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLETSDGDTFLEW | 53 |
| | | FQQRPGQSPRLLIYEVSSRFSGVPDRFSGSGSGTDFTLKI | |
| | | SRVEAEDVGVYYCMQVTHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-003 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLETSDGDTFLE | 4 |
| | LC CDR2 | EVSNRFS | 18 |
| | LC CDR3 | FQVTHDPVT | 25 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLETSDGDTFLEW | 31 |
| | | FQQRPGQSPRLLIYEVSNRFSGVPDRFSGSGSGTDFTLKI | |
| | | SRVEAEDVGVYYCFQVTHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLIVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLETSDGDTFLEW | 54 |
| | | FQQRPGQSPRLLIYEVSNRFSGVPDRFSGSGSGTDFTLKI | |
| | | SRVEAEDVGVYYCFQVTHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-004 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLESSDGDTFLE | 14 |
| | LC CDR2 | DVSTRFS | 19 |
| | LC CDR3 | FQVTHDPVT | 25 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLESSDGDTFLEW | 32 |
| | | FQQRPGQSPRLLIYDVSTRFSGVPDRFSGSGSGTDFTLKI | |
| | | SRVEAEDVGVYYCFQVTHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLIVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLESSDGDTFLEW | 55 |
| | | FQQRPGQSPRLLIYDVSTRFSGVPDRFSGSGSGTDFTLKI | |
| | | SRVEAEDVGVYYCFQVTHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-005 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLEESDGYTFLE | 15 |
| | LC CDR2 | DVSERFS | 20 |
| | LC CDR3 | FQATYDPLT | 26 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEESDGYTFLEW | 33 |
| | | FQQRPGQSPRLLIYDVSERFSGVPDRFSGSGSGTDFTLKI | |
| | | SRVEAEDVGVYYCFQATYDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLIVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLESSDGDTFLEW | 56 |
| | | FQQRPGQSPRLLIYDVSTRFSGVPDRFSGSGSGTDFTLKI | |
| | | SRVEAEDVGVYYCFQVTHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-006 | HC CDR1 | YYGMN | 9 |
| | HC CDR2 | MIYYDSSEKHYADSVKG | 2 |
| | HC CDR3 | GTTPDY | 3 |
| | LC CDR1 | RSSQSLEDSDGGTFLE | 16 |
| | LC CDR2 | DVSSRES | 21 |
| | LC CDR3 | FQATHDPLT | 27 |
| | VH | EVQLVESGGGLVQPGGSLRLSCAASGFTFSYYGMNWIRQA | 34 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEDSDGGTFLEW | 35 |
| | | FQQRPGQSPRLLIYDVSSRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSYYGMNWIRQA | 57 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSYYGMNWIRQA | 115 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLESSDGDTFLEW | 58 |
| | | FQQRPGQSPRLLIYDVSTRFSGVPDRFSGSGSGTDFTLKI | |
| | | SRVEAEDVGVYYCFQVTHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-007 | HC CDR1 | NYGMN | 1 |
| | HC CDR2 | MIYYDSSDKHYADSVKG | 10 |
| | HC CDR3 | GTTPDV | 11 |
| | LC CDR1 | RSSQSLEESDGYTFLH | 17 |
| | LC CDR2 | EVSNRFS | 18 |
| | LC CDR3 | FQATHDPVT | 28 |
| | VH | EVQLVESGGGLVQPGGSLRLSCAASGFTFSNYGMNWIRQA | 36 |
| | | PGKGLEWIGMIYYDSSDKHYADSVKGRFTISRDNSKNTLY | |
| | | LQMNSLRAEDTAVYYCAKGTTPDVWGQGTMVTVSS | |
| | VL | DVVLTQSPLSLPVTLGQPASISCRSSQSLEESDGYTFLHW | 37 |
| | | FQQRPGQSPRLLIYEVSNRFSGVPDRFSGSGSGTDFTLKI | |
| | | SRVEAEDVGVYYCFQATHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSFNRGEC | |
| | Heavy Chain | EVQLVESGGGLVQPGGSLRLSCAASGFTFSNYGMNWIRQA | 59 |
| | (with or without | PGKGLEWIGMIYYDSSDKHYADSVKGRFTISRDNSKNTLY | |
| | the lysine residue | LQMNSLRAEDTAVYYCAKGTTPDVWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 116 |
| | | PGKGLEWIGMIYYDSSDKHYADSVKGRFTISRDNSKNTLY | |
| | | LQMNSLRAEDTAVYYCAKGTTPDVWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEESDGYTFLHW | 60 |
| | | FQQRPGQSPRLLIYEVSNRFSGVPDRFSGSGSGTDFTLKI | |
| | | SRVEAEDVGVYYCFQATHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-008 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLEESDGYTFLH | 17 |
| | LC CDR2 | EVSTRFS | 5 |
| | LC CDR3 | FQATHDPLT | 27 |
| | VH | EVQLVESGGGLVQPGGSLRLSCAASGFTFSNYGMNWVRQA | 38 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEESDGYTFLHW | 39 |
| | | FQQRPGQSPRLLIYEVSTRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 61 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 117 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEESDGYTFLHW | 62 |
| | | FQQRPGQSPRLLIYEVSTRFSGVPDRFSGSGSGTDFTLKI | |
| | | SRVEAEDVGVYYCFQATHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-008- | HC CDR1 | NYGMN | 1 |
| QL | HC CDR2 | MIYYDSSEKHYADSVKG | 2 |
| | HC CDR3 | GTTPDY | 3 |
| | LC CDR1 | RSSQSLEESDGYTFLH | 17 |
| | LC CDR2 | EVSTRFS | 5 |
| | LC CDR3 | FQATHDPLT | 27 |
| | VH | EVQLVESGGGLVQPGGSLRLSCAASGFTFSNYGMNWVRQA | 38 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEESDGYTFLHW | 39 |
| | | FQQRPGQSPRLLIYEVSTRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 48 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDQLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVLHEALHNHYT | |
| | | QKSLSLSPGK | |
| | | ASTKGPSVFPLAPSSKSTSGGTAALGCLVKDYFPEPVTVS | 113 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDQLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVLHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSFNRGEC | |
| | Heavy Chain | EVQLVESGGGLVQPGGSLRLSCAASGFTFSNYGMNWVRQA | 63 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDQLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVLHEALHNHYTQKSLS | |
| | | LSPGK | |
| | | EVQLVESGGGLVQPGGSLRLSCAASGFTFSNYGMNWVRQA | 118 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDQLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVLHEALHNHYTQKSLS | |
| | | LSPG | |
| | Light Chain | DVVLTQSPLSLPVTLGQPASISCRSSQSLEESDGYTFLHW | 62 |
| | | FQQRPGQSPRLLIYEVSTRFSGVPDRFSGSGSGTDFTLKI | |
| | | SRVEAEDVGVYYCFQATHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSFNRGEC | |
| hHA-009 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLADSDGDTFLH | 50 |
| | LC CDR2 | AVSHRFS | 22 |
| | LC CDR3 | FQATHDPVT | 28 |
| | VH | EVQLVESGGGLVQPGGSLRLSCAASGFTFSNYGMNWVRQA | 38 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLADSDGDTFLHW | 41 |
| | | FQQRPGQSPRLLIYAVSHRFSGVPDRFSGSGSGTDFTLKI | |
| | | SRVEAEDVGVYYCFQATHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 61 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 117 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLADSDGDTFLHW | 65 |
| | | FQQRPGQSPRLLIYAVSHRFSGVPDRFSGSGSGTDFTLKI | |
| | | SRVEAEDVGVYYCFQATHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSFNRGEC | |
| hHA-010 | HC CDR1 | NYGMN | 1 |
| | HC CDR2 | MIYYDSSEKHYADSVKG | 2 |
| | HC CDR3 | GTTPDK | 12 |
| | LC CDR1 | RSSQSLEESDGYTFLE | 15 |
| | LC CDR2 | EVSHRFS | 23 |
| | LC CDR3 | FQATHDPLT | 27 |
| | VH | EVQLVESGGGLVQPGGSLRLSCAASGFTFSNYGMNWVRQA | 42 |
| | | PGKGLEWIGMIYYDSSEKHYADSVKGRFTISRDNSKNTLY | |
| | | LQMNSLRAEDTAVYYCAKGTTPDKWGQGTMVTVSS | |
| | VL | DVVLTQSPLSLPVTLGQPASISCRSSQSLEESDGYTFLEW | 43 |
| | | FQQRPGQSPRLLIYEVSHRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 66 |
| | (with or without | PGKGLEWIGMIYYDSSEKHYADSVKGRFTISRDNSKNTLY | |
| | the lysine residue | LQMNSLRAEDTAVYYCAKGTTPDKWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 119 |
| | | PGKGLEWIGMIYYDSSEKHYADSVKGRFTISRDNSKNTLY | |
| | | LQMNSLRAEDTAVYYCAKGTTPDKWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEESDGYTFLEW | 67 |
| | | FQQRPGQSPRLLIYEVSHRFSGVPDRFSGSGSGTDFTLKI | |
| | | SRVEAEDVGVYYCFQATHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-011 | HC CDR1 | NYGMN | 1 |
| | HC CDR2 | MIYYDSSEKHYADSVKG | 2 |
| | HC CDR3 | GSTPDY | 13 |
| | LC CDR1 | RSSQSLEDSDGGTFLE | 16 |
| | LC CDR2 | DVSSRFS | 21 |
| | LC CDR3 | FQATHDPLS | 29 |
| | VH | EVQLVESGGGLVQPGGSLRLSCAASGFTFSNYGMNWVRQA | 44 |
| | | PGKGLEWIGMIYYDSSEKHYADSVKGRFTISRDNSKNTLY | |
| | | LQMNSLRAEDTAVYYCAKGSTPDYWGQGTMVTVSS | |
| | VL | DVVLTQSPLSLPVTLGQPASISCRSSQSLEDSDGGTFLEW | 45 |
| | | FQQRPGQSPRLLIYDVSSRFSGVPDRFSGSGSGTDFTLKI | |
| | | SRVEAEDVGVYYCFQATHDPLSFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at C-terminal of | YVDGVEVHNAKTKPREEQYNSTYRVVSVLTVLHQDWLNGK | |
| | heavy chain | EYKCKVSNKALPAPIEKTISKAKGQPREPQVYTLPPSREE | |
| | constant region) | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 68 |
| | (with or without | PGKGLEWIGMIYYDSSEKHYADSVKGRFTISRDNSKNTLY | |
| | the lysine residue | LQMNSLRAEDTAVYYCAKGSTPDYWGQGTMVTVSSASTKG | |
| | at C-terminal of | PSVFPLAPSSKSTSGGTAALGCLVKDYFPEPVTVSWNSGA | |
| | heavy chain) | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 120 |
| | | PGKGLEWIGMIYYDSSEKHYADSVKGRFTISRDNSKNTLY | |
| | | LQMNSLRAEDTAVYYCAKGSTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEDSDGGTFLEW | 69 |
| | | FQQRPGQSPRLLIYDVSSRFSGVPDRFSGSGSGTDFTLKI | |
| | | SRVEAEDVGVYYCFQATHDPLSFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |

In some embodiments, the N-terminal of the heavy chain of the anti-HJV antibody described herein is glutamic acid (E). In some embodiments, the glutamic acid can cyclize spontaneously to pyroglutamic acid by post-translational modification. Spontaneous cyclization of glutamic acid to pyroglutamic acid has been previously described, e.g., Chelius et al., Formation of Pyroglutamic Acid From N-terminal Glutamic Acid in Immunoglobulin Gamma Antibodies, Anal Chem. 2006; 78 (7): 2370-2376. In some embodiments, the N-terminal of the heavy chain of the anti-HJV antibody described herein is a pyroglutamic acid. In some embodiments, the anti-HJV antibodies having N-terminal pyroglutamic acid are impurities in the population of anti-HJV antibodies (e.g., less than 5%, less than 4%, less than 3%, less than 2%, less than 1%, less than 0.5%, less than 0.1%, less than 0.05%, or less than 0.01%) in the population of anti-HJV antibody. In some embodiments, the population of the anti-HJV antibodies comprises a mixture of anti-HJV antibodies having glutamic acid or pyroglutamic acid at the N-terminal of the heavy chain.

In some embodiments, the anti-HJV antibodies of the present disclosure comprises one or more of the HC CDRs (e.g., HC CDR1, HC CDR2, or HC CDR3) amino acid sequences from any one of the anti-HJV antibodies selected from Table 1. In some embodiments, the anti-HJV antibodies of the present disclosure comprise the HC CDR1, HC CDR2, and HC CDR3 as provided for any one of the antibodies elected from Table 1. In some embodiments, the anti-HJV antibodies of the present disclosure comprises one or more of the LC CDRs (e.g., LC CDR1, LC CDR2, or LC CDR3) amino acid sequences from any one of the anti-HJV antibodies selected from Table 1. In some embodiments, the anti-HJV antibodies of the present disclosure comprise the LC CDR1, LC CDR2, and LC CDR3 as provided for any one of the anti-HJV antibodies selected from Table 1.

In some embodiments, the anti-HIV antibodies of the present disclosure comprises the HC CDR1, HC CDR2, HC CDR3, LC CDR1, LC CDR2, and LC CDR3 as provided for any one of the anti-HJV antibodies selected from Table 1. In some embodiments, antibody heavy and light chain CDR3 domains may play a particularly important role in the binding specificity/affinity of an antibody for an antigen. Accordingly, the anti-HIV antibodies of the disclosure may include at least the heavy and/or light chain CDR3s of any one of the anti-HJV antibodies selected from Table 1.

In some embodiments, the isolated anti-HJV antibody comprises a heavy chain variable region that comprises a heavy chain CDR1 (HC CDR1), a heavy chain CDR2 (HC CDR2), and a heavy chain CDR3 (HC CDR3).

In some embodiments, following the Kabat definition, the HC CDR1 may comprise the amino acid sequence of X₁YGMN (SEQ ID NO: 105), in which X₁ can be N or Y. Alternatively or in addition, the HC CDR2 may comprise the amino acid sequence of MIYYDSSX₂KHYADSVKG (SEQ ID NO: 106), in which X₂ can be E or D. Alternatively or in addition, the HC CDR3 may comprise the amino acid sequence of GX₃TPDX₄ (SEQ ID NO: 107), in which X₃ can be T or S, and X₄ can be Y, V, or K.

In some embodiments, following the Kabat definition, the anti-HJV antibody may comprise a light chain variable region that comprises a light chain CDR1 (LC CDR1), a light chain CDR2 (LC CDR2), and a light chain CDR3 (LC CDR3). In some embodiments, the LC CDR1 may comprise the amino acid sequence of RSSQSLX₅X₆SDGX₇TFLX₈ (SEQ ID NO: 108), in which X₅ can be A or E, X₆ can be T, S, E, or D, X₇ can be D, Y, or G, and X₈ can be E or H. Alternatively or in addition, the LC CDR2 may comprise the amino acid sequence of X₉VSX₁₀RFS (SEQ ID NO: 109), in which X₉ can be E, D or A, and X₁₀ can be N, S, T, E or H. Alternatively or in addition, the LC CDR3 may comprise the amino acid sequence of X₁₁QX₁₂TX₁₃DPX₁₄X₁₅ (SEQ ID NO: 110), in which X₁₁ can be For M, X₁₂ can be V or A, X₁₃ can be H or Y, X₁₄ can be M, L or V, and X₁₅ can be T or S.

Also within the scope of the present disclosure are functional variants of any of the exemplary anti-HJV antibodies as disclosed herein. A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, anti-inflammatory activity, or a combination thereof) as the reference antibody.

In some embodiments, any of the anti-HJV antibodies of the disclosure have one or more CDRs (e.g., HC CDR or LC CDR) sequences substantially similar to any of the HC CDR1, HC CDR2, HC CDR3, LC CDR1, LC CDR2, and/or LC CDR3 sequences from one of the anti-HJV antibodies selected from Table 1. In some embodiments, the position of one or more CDRs along the V_{H} (e.g., HC CDR1, HC CDR2, or HC CDR3) and/or VL (e.g., LC CDR1, LC CDR2, or LC CDR3) region of an antibody described herein can vary by one, two, three, four, five, or six amino acid positions so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the V_{H} (e.g., HC CDR1, HC CDR2, or HC CDR3) and/or VL (e.g., LC CDR1, LC CDR2, or LC CDR3) region of an antibody described herein can vary (e.g., be shorter or longer) by one, two, three, four, five, or more amino acids, so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived).

Accordingly, in some embodiments, a HC CDR1, HC CDR2, HC CDR3, LC CDR1, LC CDR2, and/or LC CDR3 described herein may be one, two, three, four, five or more amino acids shorter than one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, a HC CDR1, HC CDR2, HC CDR3, LC CDR1, LC CDR2, and/or LC CDR3 described herein may be one, two, three, four, five or more amino acids longer than one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to hemojuvelin (e.g., human hemojuvelin) is maintained, for example, using binding assays and conditions described in the art.

In some examples, any of the anti-HJV antibodies of the disclosure have one or more CDR (e.g., HC CDR or LC CDR) sequences substantially similar to any one of the anti-HJV antibodies selected from Table 1. For example, the antibodies may include one or more CDR sequence(s) from any of the anti-HJV antibodies selected from Table 1 containing up to 5, 4, 3, 2, or 1 amino acid residue variations as compared to the corresponding CDR region in any one of the CDRs provided herein (e.g., CDRs from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a hemojuvelin protein (e.g., a human hemojuvelin protein), for example, as determined based on a crystal structure. Some aspects of the disclosure provide anti-HJV antibodies that comprise one or more of the heavy chain variable (VH) and/or light chain variable (VL) domains provided herein. In some embodiments, any of the V_{H} domains provided herein include one or more of the HC CDR sequences (e.g., HC CDR1, HC CDR2, and HC CDR3) provided herein, for example, any of the CDR-H sequences provided in any one of the anti-HIV selected from Table 1. In some embodiments, any of the VL domains provided herein include one or more of the CDR-L sequences (e.g., LC CDR1, LC CDR2, and LC CDR3) provided herein, for example, any of the LC CDR sequences provided in any one of the anti-HJV antibodies selected from Table 1.

In some embodiments, the anti-HJV antibodies of the disclosure include any antibody that includes a heavy chain variable domain and/or a light chain variable domain of any one of the anti-HJV antibodies selected from Table 1, and variants thereof. In some embodiments, anti-HJV antibodies of the disclosure include any antibody that includes the heavy chain variable and light chain variable pairs of any anti-HIV antibodies selected from Table 1.

Aspects of the disclosure provide anti-HIV antibodies having a heavy chain variable (VH) and/or a light chain variable (VL) domain amino acid sequence homologous to any of those described herein. In some embodiments, the anti-HJV antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any one of the anti-HJV antibodies selected from Table 1. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, any of the anti-HJV antibodies provided herein comprise a heavy chain variable sequence and a light chain variable sequence that comprises a framework sequence that is at least 75%, 80%, 85%, 90%, 95%, 98%, or 99% identical to the framework sequence of any anti-HJV antibodies selected from Table 1.

In some embodiments, the anti-HJV antibody of the present disclosure is a humanized antibody (e.g., a humanized variant containing one or more CDRs of Table 1). In some embodiments, the anti-HJV antibody of the present disclosure comprises a HC CDR1, a HC CDR2, a HC CDR3, a LC CDR1, a LC CDR2, and a LC CDR3 that are the same as the HC CDR1, HC CDR2, HC CDR3, LC CDR1, LC CDR2, and LC CDR3 shown in Table 1, and comprises a humanized heavy chain variable region and/or a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 1) into the human variable domains (e.g., IGKV1-NL1*01 and IGHV1-3*01 human variable domain). In some embodiments, the anti-HJV antibody of the present disclosure is a humanized variant comprising one or more amino acid substitutions (e.g., in the V_{H} framework region) as compared with any one of the VHs listed in Table 1, and/or one or more amino acid substitutions (e.g., in the VL framework region) as compared with any one of the VLs listed in Table 1.

In some embodiments, the anti-HJV antibody of the present disclosure is a humanized antibody comprising a V_{H} containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the V_{H} of any of the anti-HJV antibodies listed in Table 1. Alternatively or in addition, the anti-HJV antibody of the present disclosure is a humanized antibody comprising a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-HJV antibodies listed in Table 1.

In some embodiments, the anti-HIV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 8.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HIV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 8.

In some embodiments, the anti-HIV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HIV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 7. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 8.

In some embodiments, the anti-HIV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 30.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 49, and a LC CDR3 having the amino acid sequence of SEQ ID NO: 24.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 49, and LC CDR3 having the amino acid sequence of SEQ ID NO: 24.

In some embodiments, the anti-HIV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 49, and LC CDR3 having the amino acid sequence of SEQ ID NO: 24.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 49; and/or a LC CDR3 having no more than 3 amino acid variations (e.g., no more than 3, 2, or 1 amino acid variation) as compared with the LC CDR3 having the amino acid sequence of SEQ ID NO: 24.

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 30.

In some embodiments, the anti-HIV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 30.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 7. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 30.

In some embodiments, the anti-HJV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 31.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 18, and a LC CDR3 having the amino acid sequence of SEQ ID NO: 25.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 18, and LC CDR3 having the amino acid sequence of SEQ ID NO: 25.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 18, and LC CDR3 having the amino acid sequence of SEQ ID NO: 25.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HIV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 18; and/or a LC CDR3 having no more than 3 amino acid variations (e.g., no more than 3, 2, or 1 amino acid variation) as compared with the LC CDR3 having the amino acid sequence of SEQ ID NO: 25

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 31

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 31

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 7. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 31.

In some embodiments, the anti-HJV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 32.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 14, a LC CDR2 having the amino acid sequence of SEQ ID NO: 19, and a LC CDR3 having the amino acid sequence of SEQ ID NO: 25.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 14, LC CDR2 having the amino acid sequence of SEQ ID NO: 19, and LC CDR3 having the amino acid sequence of SEQ ID NO: 25.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 14, LC CDR2 having the amino acid sequence of SEQ ID NO: 19, and LC CDR3 having the amino acid sequence of SEQ ID NO: 25.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 14; a LC CDR2 having no more than 3 amino acid variations (e.g., no more than 3, 2, or 1 amino acid variation) as compared with the LC CDR2 having the amino acid sequence of SEQ ID NO: 19; and/or a LC CDR3 having no more than 3 amino acid variations (e.g., no more than 3, 2, or 1 amino acid variation) as compared with the LC CDR3 having the amino acid sequence of SEQ ID NO: 25.

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 32.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 32.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 7. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 32.

In some embodiments, the anti-HJV antibody of the present disclosure comprises a HC CDR1, HC CDR2 and HC CDR3 of a heavy chain variable domain having the amino acid sequence of SEQ ID NO: 7. Alternatively or in addition, the anti-HIV antibody of the present disclosure comprises a LC CDR1, LC CDR2 and LC CDR3 of a light chain variable domain having the amino acid sequence of SEQ ID NO: 33.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 15, a LC CDR2 having the amino acid sequence of SEQ ID NO: 20, and a LC CDR3 having the amino acid sequence of SEQ ID NO: 26.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 15, LC CDR2 having the amino acid sequence of SEQ ID NO: 20, and LC CDR3 having the amino acid sequence of SEQ ID NO: 26.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 15, LC CDR2 having the amino acid sequence of SEQ ID NO: 20, and LC CDR3 having the amino acid sequence of SEQ ID NO: 26.

In some embodiments, the anti-HIV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 15; a LC CDR2 having no more than 3 amino acid variations (e.g., no more than 3, 2, or 1 amino acid variation) as compared with the LC CDR2 having the amino acid sequence of SEQ ID NO: 20; and/or a LC CDR3 having no more than 3 amino acid variations (e.g., no more than 3, 2, or 1 amino acid variation) as compared with the LC CDR3 having the amino acid sequence of SEQ ID NO: 26.

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 33.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 33.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 7. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 33.

In some embodiments, the anti-HJV antibody of the present disclosure comprises a HC CDR1, HC CDR2 and HC CDR3 of a heavy chain variable domain having the amino acid sequence of SEQ ID NO: 34. Alternatively or in addition, the anti-HJV antibody of the present disclosure comprises a LC CDR1, LC CDR2 and LC CDR3 of a light chain variable domain having the amino acid sequence of SEQ ID NO: 35.

In some embodiments, according to the Kabat definition system, the anti-HJV antibody of the present disclosure comprises a HC CDR1 having the amino acid sequence of SEQ ID NO: 9, a HC CDR2 having the amino acid sequence of SEQ ID NO: 2, a HC CDR3 having the amino acid sequence of SEQ ID NO: 3, a LC CDR1 having the amino acid sequence of SEQ ID NO: 16, a LC CDR2 having the amino acid sequence of SEQ ID NO: 21, and a LC CDR3 having the amino acid sequence of SEQ ID NO: 27.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 9, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 16, LC CDR2 having the amino acid sequence of SEQ ID NO: 21, and LC CDR3 having the amino acid sequence of SEQ ID NO: 27.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 9, HC CDR2 having the amino acid sequence of SEQ ID NO: 2, and HC CDR3 having the amino acid sequence of SEQ ID NO: 3. Alternatively or in addition, the anti-HIV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 16, LC CDR2 having the amino acid sequence of SEQ ID NO: 21, and LC CDR3 having the amino acid sequence of SEQ ID NO: 27.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 9; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 16; a LC CDR2 having no more than 3 amino acid variations (e.g., no more than 3, 2, or 1 amino acid variation) as compared with the LC CDR2 having the amino acid sequence of SEQ ID NO: 21; and/or a LC CDR3 having no more than 3 amino acid variations (e.g., no more than 3, 2, or 1 amino acid variation) as compared with the LC CDR3 having the amino acid sequence of SEQ ID NO: 27.

In some embodiments, the anti-HJV antibody of the present disclosure comprises a VH comprising the amino acid sequence of SEQ ID NO: 34. Alternatively or in addition, the anti-HJV antibody of the present disclosure comprises a VL comprising the amino acid sequence of SEQ ID NO: 35.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 34. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 35.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 34. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 35.

In some embodiments, the anti-HJV antibody of the present disclosure comprises a HC CDR1, HC CDR2 and HC CDR3 of a heavy chain variable domain having the amino acid sequence of SEQ ID NO: 36. Alternatively or in addition, the anti-HJV antibody of the present disclosure comprises a LC CDR1, LC CDR2 and LC CDR3 of a light chain variable domain having the amino acid sequence of SEQ ID NO: 37.

In some embodiments, according to the Kabat definition system, the anti-HIV antibody of the present disclosure comprises a HC CDR1 having the amino acid sequence of SEQ ID NO: 1, a HC CDR2 having the amino acid sequence of SEQ ID NO: 10, a HC CDR3 having the amino acid sequence of SEQ ID NO: 11, a LC CDR1 having the amino acid sequence of SEQ ID NO: 17, a LC CDR2 having the amino acid sequence of SEQ ID NO: 18, and a LC CDR3 having the amino acid sequence of SEQ ID NO: 28.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 10, and HC CDR3 having the amino acid sequence of SEQ ID NO: 11. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 17, LC CDR2 having the amino acid sequence of SEQ ID NO: 18, and LC CDR3 having the amino acid sequence of SEQ ID NO: 28.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 10, and HC CDR3 having the amino acid sequence of SEQ ID NO: 11. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 17, LC CDR2 having the amino acid sequence of SEQ ID NO: 18, and LC CDR3 having the amino acid sequence of SEQ ID NO: 28.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 10; and/or a HC CDR3 having no more than 3 amino acid variations (e.g., no more than 3, 2, or 1 amino acid variation) as compared with the HC CDR3 having the amino acid sequence of SEQ ID NO: 11. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 17; a LC CDR2 having no more than 3 amino acid variations (e.g., no more than 3, 2, or 1 amino acid variation) as compared with the LC CDR2 having the amino acid sequence of SEQ ID NO: 18; and/or a LC CDR3 having no more than 3 amino acid variations (e.g., no more than 3, 2, or 1 amino acid variation) as compared with the LC CDR3 having the amino acid sequence of SEQ ID NO: 28.

In some embodiments, the anti-HJV antibody of the present disclosure comprises a VH comprising the amino acid sequence of SEQ ID NO: 36. Alternatively or in addition, the anti-HJV antibody of the present disclosure comprises a VL comprising the amino acid sequence of SEQ ID NO: 37.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 36. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 37.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 36. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 37.

In some embodiments, the anti-HJV antibody of the present disclosure comprises a HC CDR1, HC CDR2 and HC CDR3 of a heavy chain variable domain having the amino acid sequence of SEQ ID NO: 38. Alternatively or in addition, the anti-HJV antibody of the present disclosure comprises a LC CDR1, LC CDR2 and LC CDR3 of a light chain variable domain having the amino acid sequence of SEQ ID NO: 39.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 17, a LC CDR2 having the amino acid sequence of SEQ ID NO: 5, and a LC CDR3 having the amino acid sequence of SEQ ID NO: 27.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 17, LC CDR2 having the amino acid sequence of SEQ ID NO: 5, and LC CDR3 having the amino acid sequence of SEQ ID NO: 27.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 17, LC CDR2 having the amino acid sequence of SEQ ID NO: 5, and LC CDR3 having the amino acid sequence of SEQ ID NO: 27.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 17; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 27.

In some embodiments, the anti-HJV antibody of the present disclosure comprises a VH comprising the amino acid sequence of SEQ ID NO: 38. Alternatively or in addition, the anti-HJV antibody of the present disclosure comprises a VL comprising the amino acid sequence of SEQ ID NO: 39.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 38. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 39.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 38. Alternatively or in addition, the anti-HIV antibody of the present disclosure comprises a VL comprising an amino acid sequence that is at least 80% (e.g., 80%, 85%, 90%, 95%, 98%, or 99%) identical to the VL as set forth in SEQ ID NO: 39.

In some embodiments, the anti-HJV antibody of the present disclosure comprises a HC CDR1, HC CDR2 and HC CDR3 of a heavy chain variable domain having the amino acid sequence of SEQ ID NO: 38. Alternatively or in addition, the anti-HJV antibody of the present disclosure comprises a LC CDR1, LC CDR2 and LC CDR3 of a light chain variable domain having the amino acid sequence of SEQ ID NO: 41.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 50, a LC CDR2 having the amino acid sequence of SEQ ID NO: 22, and a LC CDR3 having the amino acid sequence of SEQ ID NO: 28.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 50, LC CDR2 having the amino acid sequence of SEQ ID NO: 22, and LC CDR3 having the amino acid sequence of SEQ ID NO: 28.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HIV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 50, LC CDR2 having the amino acid sequence of SEQ ID NO: 22, and LC CDR3 having the amino acid sequence of SEQ ID NO: 28.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HIV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 50; a LC CDR2 having no more than 3 amino acid variations (e.g., no more than 3, 2, or 1 amino acid variation) as compared with the LC CDR2 having the amino acid sequence of SEQ ID NO: 22; and/or a LC CDR3 having no more than 3 amino acid variations (e.g., no more than 3, 2, or 1 amino acid variation) as compared with the LC CDR3 having the amino acid sequence of SEQ ID NO: 28.

In some embodiments, the anti-HJV antibody of the present disclosure comprises a VH comprising the amino acid sequence of SEQ ID NO: 38. Alternatively or in addition, the anti-HJV antibody of the present disclosure comprises a VL comprising the amino acid sequence of SEQ ID NO: 41.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 38. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 41.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 38. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 41.

In some embodiments, the anti-HJV antibody of the present disclosure comprises a HC CDR1, HC CDR2 and HC CDR3 of a heavy chain variable domain having the amino acid sequence of SEQ ID NO: 42. Alternatively or in addition, the anti-HJV antibody of the present disclosure comprises a LC CDR1, LC CDR2 and LC CDR3 of a light chain variable domain having the amino acid sequence of SEQ ID NO: 43.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 12, a LC CDR1 having the amino acid sequence of SEQ ID NO: 15, a LC CDR2 having the amino acid sequence of SEQ ID NO: 23, and a LC CDR3 having the amino acid sequence of SEQ ID NO: 27.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 12. “Collectively.” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 15, LC CDR2 having the amino acid sequence of SEQ ID NO: 23, and LC CDR3 having the amino acid sequence of SEQ ID NO: 27.

In some embodiments, the anti-HIV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 12. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 15, LC CDR2 having the amino acid sequence of SEQ ID NO: 23, and LC CDR3 having the amino acid sequence of SEQ ID NO: 27.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 12. Alternatively or in addition, the anti-HIV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 15; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 27.

In some embodiments, the anti-HJV antibody of the present disclosure comprises a VH comprising the amino acid sequence of SEQ ID NO: 42. Alternatively or in addition, the anti-HJV antibody of the present disclosure comprises a VL comprising the amino acid sequence of SEQ ID NO: 43.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 42. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 43.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 42. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 43.

In some embodiments, the anti-HIV antibody of the present disclosure comprises a HC CDR1, HC CDR2 and HC CDR3 of a heavy chain variable domain having the amino acid sequence of SEQ ID NO: 44. Alternatively or in addition, the anti-HJV antibody of the present disclosure comprises a LC CDR1, LC CDR2 and LC CDR3 of a light chain variable domain having the amino acid sequence of SEQ ID NO: 45.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 13, a LC CDR1 having the amino acid sequence of SEQ ID NO: 16, a LC CDR2 having the amino acid sequence of SEQ ID NO: 21, and a LC CDR3 having the amino acid sequence of SEQ ID NO: 29.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 13. “Collectively.” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 16, LC CDR2 having the amino acid sequence of SEQ ID NO: 21, and LC CDR3 having the amino acid sequence of SEQ ID NO: 29.

In some embodiments, the anti-HJV antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 13. Alternatively or in addition, the anti-HJV antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 16, LC CDR2 having the amino acid sequence of SEQ ID NO: 21, and LC CDR3 having the amino acid sequence of SEQ ID NO: 29.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 13. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 16; a LC CDR2 having no more than 3 amino acid variations (e.g., no more than 3, 2, or 1 amino acid variation) as compared with the LC CDR2 having the amino acid sequence of SEQ ID NO: 21; and/or a LC CDR3 having no more than 3 amino acid variations (e.g., no more than 3, 2, or 1 amino acid variation) as compared with the LC CDR3 having the amino acid sequence of SEQ ID NO: 29.

In some embodiments, the anti-HJV antibody of the present disclosure comprises a VH comprising the amino acid sequence of SEQ ID NO: 44. Alternatively or in addition, the anti-HIV antibody of the present disclosure comprises a VL comprising the amino acid sequence of SEQ ID NO: 45.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45.

In some embodiments, the anti-HJV antibody of the present disclosure comprises a VH comprising an amino acid sequence that is at least 80% (e.g., 80%, 85%, 90%, 95%, 98%, or 99%) identical to the V_{H} as set forth in SEQ ID NO: 44. Alternatively or in addition, the anti-HJV antibody of the present disclosure comprises a VL comprising an amino acid sequence that is at least 80% (e.g., 80%, 85%, 90%, 95%, 98%, or 99%) identical to the VL as set forth in SEQ ID NO: 45.

The CDRs of an antibody may have different amino acid sequences when different definition systems are used (e.g., the IMGT definition, the Kabat definition, or the Chothia definition). A definition system annotates each amino acid in a given antibody sequence (e.g., V_{H} or VL sequence) with a number, and numbers corresponding to the heavy chain and light chain CDRs are provided in Table 2. The CDRs listed in Table 1 are defined in accordance with the Kabat definition. One skilled in the art is able to derive the CDR sequences using the different numbering systems for the anti-HJV antibodies provided in Table 2.

TABLE 2
CDR Definitions

| | IMGT¹ | Kabat² | Chothia³ |
|---|---|---|---|
| HC CDR1 | 27-38 | 31-35 | 26-32 |
| HC CDR2 | 56-65 | 50-65 | 53-55 |
| HC CDR3 | 105-116/117 | 95-102 | 96-101 |
| LC CDR1 | 27-38 | 24-34 | 26-32 |
| LC CDR2 | 56-65 | 50-56 | 50-52 |
| LC CDR3 | 105-116/117 | 89-97 | 91-96 |

¹IMGT ®, the international ImMunoGeneTics information system ®, imgt.org, Lefranc, M.-P. et al., Nucleic Acids Res., 27:209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196:901-917 (1987))

In some embodiments, the anti-HJV antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the anti-HJV antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the anti-HJV antibody of the present disclosure comprises a VL domain and/or V_{H} domain of any one of the anti-HJV antibodies selected from Table 1, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra. The human IgG1 constant region sequences below (e.g., SEQ ID NO: 48, SEQ ID NO: 112, SEQ ID NO: 113, and SEQ ID NO: 130) are variants of (e.g., having between 1 and 5 amino acids that differ from) the heavy chain constant region sequence set forth in SEQ ID NO: 46. An example of a human IgG1 constant region is given below:

(SEQ ID NO: 103)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the heavy chain of any of the anti-HJV antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcg receptor binding (Bruhns, P., et al. (2009) and Xu, D. et al. (2000)). The mutant human IgG1 constant region is provided below (mutations bonded and underlined):

(SEQ ID NO: 130)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKIKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the heavy chain of any of the anti-HJV antibodies described herein further comprises mutations in human IgG1 constant region. For example, the introduction of T250Q and M248L substitutions. In some embodiments, such substitution may affect FcRn binding and serum half-life (WO2005047307 and WO2013063110). An exemplary IgG1 constant region comprising the LALA mutation and the QL mutation is provided below (mutations bonded and underlined):

(SEQ ID NO: 48)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDQLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVLHEALHNHYTQKSLSLSPGK

In some embodiments, during the production of the antibodies, particularly with Chinese Hamster Ovary Cells (CHO cells), it can be appreciated that the lysine at the C-terminus of the heavy chain is cleaved. Accordingly, a human IgG1 constant region within a secreted antibody can be:

(SEQ ID NO: 111)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

In some embodiments, a mutant human IgG1 comprising the LALA mutations in a secreted antibody can be:

(SEQ ID NO: 112)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

In some embodiments, a mutant human IgG1 comprising the LALA mutations and the QL mutations can be:

(SEQ ID NO: 113)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDQLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVLHEALHNHYTQKSLSLSPG

In some embodiments, the light chain of any of the anti-HIV antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 47)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other antibody heavy and light chain constant regions are well known in the art, e.g., those provided in the IMGT database (imgt.org) or at vbase2.org/vbstat), both of which are incorporated by reference herein.

In some embodiments, the anti-HJV antibody described herein comprises a heavy chain comprising any one of the V_{H} as listed in Table 1 or any variants thereof and a heavy chain constant region that is at least 80%, at least 85%, at least 90%, at least 95%, or at least 99% identical to SEQ ID NO: 46, SEQ ID NO: 48, SEQ ID NO: 112, SEQ ID NO: 113, or SEQ ID NO: 130. In some embodiments, the anti-HJV antibody described herein comprises a heavy chain comprising any one of the V_{H} as listed in Table 1 or any variants thereof and a heavy chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 46, SEQ ID NO: 48, SEQ ID NO: 112, SEQ ID NO: 113, or SEQ ID NO: 130. In some embodiments, the anti-HJV antibody described herein comprises a heavy chain comprising any one of the V_{H} as listed in Table 1 or any variants thereof and a heavy chain constant region as set forth in SEQ ID NO: 46. In some embodiments, the anti-HJV antibody described herein comprises heavy chain comprising any one of the V_{H} as listed in Table 1 or any variants thereof and a heavy chain constant region as set forth in SEQ ID NO: 48. In some embodiments, the anti-HJV antibody described herein comprises heavy chain comprising any one of the V_{H} as listed in Table 1 or any variants thereof and a heavy chain constant region as set forth in SEQ ID NO: 112. In some embodiments, the anti-HJV antibody described herein comprises heavy chain comprising any one of the V_{H} as listed in Table 1 or any variants thereof and a heavy chain constant region as set forth in SEQ ID NO: 113. In some embodiments, the anti-HIV antibody described herein comprises heavy chain comprising any one of the V_{H} as listed in Table 1 or any variants thereof and a heavy chain constant region as set forth in SEQ ID NO: 130.

In some embodiments, the anti-HJV antibody described herein comprises a light chain comprising any one of the VL as listed in Table 1 or any variants thereof and a light chain constant region that is at least 80%, at least 85%, at least 90%, at least 95%, or at least 99% identical to SEQ ID NO: 47. In some embodiments, the anti-HJV antibody described herein comprises a light chain comprising any one of the VL as listed in Table 1 or any variants thereof and a light chain constant region contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 47. In some embodiments, the anti-HJV antibody described herein comprises a light chain comprising any one of the VL as listed in Table 1 or any variants thereof and a light chain constant region set forth in SEQ ID NO: 47.

Examples of IgG heavy chain and light chain amino acid sequences of the anti-HJV antibodies described are provided in Table 1 above.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51, 57, 59, 61, 63, 66, 68, 114, 115, 116, 117, 118, 119 or 120. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 52, 53, 54, 55, 56, 58, 60, 62, 65, 67 or 69. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 51, 57, 59, 61, 63, 66, 68, 114, 115, 116, 117, 118, 119 or 120. Alternatively or in addition, the anti-HIV antibody described herein comprises a light chain comprising an amino acid sequence that is at least 80% (e.g., 80%, 85%, 90%, 95%, 98%, or 99%) identical to any one of SEQ ID NOS: 52, 53, 54, 55, 56, 58, 60, 62, 65, 67 or 69. In some embodiments, the anti-HJV antibody described herein comprises a heavy chain comprising the amino acid sequence of any one of SEQ ID NOs: 51, 57, 59, 61, 63, 66, 68, 114, 115, 116, 117, 118, 119 or 120. Alternatively or in addition, the anti-HJV antibody described herein comprises a light chain comprising the amino acid sequence of any one of SEQ ID NOs: 52, 53, 54, 55, 56, 58, 60, 62, 65, 67 or 69.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 52. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 51 or 114. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 52. In some embodiments, the anti-HIV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 52.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 53. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 51 or 114. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 53. In some embodiments, the anti-HJV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 53.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 54. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 51 or 114. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 54. In some embodiments, the anti-HJV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 54.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 55. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 51 or 114. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 55. In some embodiments, the anti-HJV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 55.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 56. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 51 or 114. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 56. In some embodiments, the anti-HIV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 56.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 57 or 115. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 58. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 57 or 115. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 58. In some embodiments, the anti-HJV antibody described herein comprises a heavy chain comprising the amino acid sequence of any one of SEQ ID NOs: 57 or 115. Alternatively or in addition, the anti-HJV antibody described herein comprises a light chain comprising the amino acid sequence of any one of SEQ ID NOs: 58.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 59 or 116. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 60. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 59 or 116. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 60. In some embodiments, the anti-HJV antibody described herein comprises a heavy chain comprising the amino acid sequence of any one of SEQ ID NOs: 59 or 116. Alternatively or in addition, the anti-HJV antibody described herein comprises a light chain comprising the amino acid sequence of any one of SEQ ID NOs: 60.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 61 or 117. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 62. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 61 or 117. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 62. In some embodiments, the anti-HJV antibody described herein comprises a heavy chain comprising the amino acid sequence of any one of SEQ ID NOs: 61 or 117. Alternatively or in addition, the anti-HJV antibody described herein comprises a light chain comprising the amino acid sequence of any one of SEQ ID NOs: 62.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 63 or 118. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 62. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 63 or 118. Alternatively or in addition, the anti-HIV antibody described herein comprises a light chain comprising an amino acid sequence that is at least 80% (e.g., 80%, 85%, 90%, 95%, 98%, or 99%) identical to any one of SEQ ID NOs: 62. In some embodiments, the anti-HJV antibody described herein comprises a heavy chain comprising the amino acid sequence of any one of SEQ ID NOs: 63 or 118. Alternatively or in addition, the anti-HJV antibody described herein comprises a light chain comprising the amino acid sequence of any one of SEQ ID NOs: 62.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 61 or 117. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 65. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 61 or 117. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 65. In some embodiments, the anti-HJV antibody described herein comprises a heavy chain comprising the amino acid sequence of any one of SEQ ID NOs: 61 or 117. Alternatively or in addition, the anti-HJV antibody described herein comprises a light chain comprising the amino acid sequence of any one of SEQ ID NOs: 65.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 66 or 119. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 67. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 66 or 119. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 67. In some embodiments, the anti-HJV antibody described herein comprises a heavy chain comprising the amino acid sequence of any one of SEQ ID NOs: 66 or 119. Alternatively or in addition, the anti-HJV antibody described herein comprises a light chain comprising the amino acid sequence of any one of SEQ ID NOs: 67.

In some embodiments, the anti-HIV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 68 or 120. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 69. In some embodiments, the anti-HJV antibody described herein comprises a heavy chain comprising an amino acid sequence that is at least 80% (e.g., 80%, 85%, 90%, 95%, 98%, or 99%) identical to any one of SEQ ID NOs: 68 or 120. Alternatively or in addition, the anti-HJV antibody described herein comprises a light chain comprising an amino acid sequence that is at least 80% (e.g., 80%, 85%, 90%, 95%, 98%, or 99%) identical to any one of SEQ ID NOs: 69. In some embodiments, the anti-HJV antibody described herein comprises a heavy chain comprising the amino acid sequence of any one of SEQ ID NOs: 68 or 120. Alternatively or in addition, the anti-HJV antibody described herein comprises a light chain comprising the amino acid sequence of any one of SEQ ID NOs: 69.

The anti-HJV antibodies described herein can be in any antibody form, including, but not limited to, intact (i.e., full-length) antibodies, antigen-binding fragments thereof (such as Fab, F′ab′), F′ab′)2, Fv), single chain antibodies, bi-specific antibodies, or nanobodies. In some embodiments, the anti-HJV antibody described herein is a scFv. In some embodiments, the anti-HJV antibody described herein is a scFv-Fab (e.g., scFv fused to a portion of a constant region).

In some embodiments, conservative mutations can be introduced into antibody sequences (e.g., CDRs or framework sequences) at positions where the residues are not likely to be involved in interacting with a target antigen (e.g., hemojuvelin), for example, as determined based on a crystal structure. In some embodiments, one, two or more mutations (e.g., amino acid substitutions) are introduced into the Fc region of an anti-HJV antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (e.g., an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (e.g., an Fc or hinge-Fc domain fragment) to decrease the half-life of the anti-HJV antibody in vivo. In some embodiments, one, two or more amino acid mutations (i.e., substitutions, insertions or deletions) are introduced into an IgG constant domain, or FcRn-binding fragment thereof (e.g., an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine(S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see D'Il'Acqua W F et al., (2006) J Biol Chem 281:23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-HJV antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. Sec, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276:6591-604).

In some embodiments, one or more amino in the constant region of an anti-HJV antibody described herein can be replaced with a different amino acid residue such that the antibody has altered Clq binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind hemojuvelin, such that the variant, CDR-grafted, chimeric, humanized, or composite antibody has at least 50%, at least 60%, at least 70%, at least 80%, at least 90%, at least 95% or more binding to hemojuvelin relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody.” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-HJV antibodies described herein may comprise a signal peptide in the heavy and/or light chain sequence (e.g., a N-terminal signal peptide). In some embodiments, the anti-HJV antibody described herein comprises any one of the V_{H} and VL sequences, any one of the IgG heavy chain and light chain sequences, or any one of the F′ab′) heavy chain and light chain sequences described herein, and further comprises a signal peptide (e.g., a N-terminal signal peptide). In some embodiments, the signal peptide comprises the amino acid sequence of MEFGLSWLFLVAILKGVQC (SEQ ID NO: 104).

### III. Preparation of the Anti-HJV Antibodies

Antibodies capable of binding hemojuvelin as described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., HJV) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, CA) and HuMAb-Mouse® and TC Mouse™ from Medarex, Inc. (Princeton, NJ) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′) 2 fragments can be produced by pepsin digestion of an antibody molecule, and Fab fragments that can be generated by reducing the disulfide bridges of F(ab′) 2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as an exemplary source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv clones specific to HJV can be identified from the library following routine procedures. Positive clones can be subjected to further screening to identify those that has high HJV binding affinity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. Alternatively, competition assays can be performed using other antibodies known to bind to the same antigen to determine whether an antibody binds to the same epitope as the other antibodies. Competition assays are well known to those of skill in the art.

In some examples, an anti-HJV antibody is prepared by recombinant technology as exemplified below. Nucleic acids encoding the heavy and light chain of an anti-HJV antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA. 89:5547-5551 (1992)] combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and Co1E1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies (e.g., the nucleic acid coding sequence listed in Table 3) may be introduced into suitable host cells for producing the antibodies. Non-limiting examples of the host cells include Chinese hamster ovary (CHO) cells, dhfr-CHO cell, human embryonic kidney (HEK)-293 cells, verda reno (VERO) cells, nonsecreting null (NS0) cells, human embryonic retinal (PER.C6) cells. Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells. In some embodiments, the host cell expressing the anti-HIV antibodies are CHO cells. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody. In some embodiments, the host cell comprises the nucleic acid encoding the heavy chain of the anti-HJV antibody. In some embodiments, the host cell comprises the nucleic acid encoding the light chain of the anti-HJV antibody. In some embodiments, the host cell comprises the nucleic acid encoding the heavy chain and the nucleic acid encoding the light chain.

In some embodiments, methods for preparing an antibody described herein involve a recombinant expression vector that encodes both the heavy chain and the light chain of an anti-HJV antibody, as also described herein. The recombinant expression vector can be introduced into a suitable host cell (e.g., a dhfr-CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-HJV antibody and the other encoding the light chain of the anti-HJV antibody. Both of the two recombinant expression vectors can be introduced into a suitable host cell (e.g., dhfr-CHO cell) by a conventional method, e.g., calcium phosphate-mediated transfection.

Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-HJV antibody as described herein (e.g., as provided in Table 3), vectors (e.g., expression vectors) containing such; and host cells comprising the vectors are within the scope of the present disclosure.

TABLE 3
Nucleic acids Sequences encoding the VH/VL of anti-HJV
antibodies listed in Table 1

| humanized HA | | | SEQ ID |
|---|---|---|---|
| Antibody | | Nucleic Acid Sequences | NO: |
| hHA-001 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 72 |
| | | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAACGAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCACGACCCC | |
| | | ATGACGTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 73 |
| | Chain | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAACGAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCACGACCCC | |
| | | ATGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-002 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 74 |
| | | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTATGCAAGTCACCCACGACCCC | |
| | | CTGACCTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 75 |
| | Chain | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTATGCAAGTCACCCACGACCCC | |
| | | CTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-003 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 76 |
| | | ACCAGCCTCAATTTCATGCAGATCATCACAATCACTTGAGACCAGCGACG | |
| | | GAGATACTTTTCTTGAGTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGTCACCCACGACCCC | |
| | | GTCACGTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 77 |
| | Chain | ACCAGCCTCAATTTCATGCAGATCATCACAATCACTTGAGACCAGCGACG | |
| | | GAGATACTTTTCTTGAGTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGTCACCCACGACCCC | |
| | | GTCACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-004 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 78 |
| | | ACCAGCCTCAATTTCATGCAGATCATCACAATCACTTGAGTCCAGCGACG | |
| | | GAGACACTTTTCTTGAGTGGTTCCAACAAAGACCCGGACAAAGCCCACGC | |
| | | CTGCTTATTTACGATGTATCAACTAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCATGACCCC | |
| | | GTGACCTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 79 |
| | Chain | ACCAGCCTCAATTTCATGCAGATCATCACAATCACTTGAGTCCAGCGACG | |
| | | GAGACACTTTTCTTGAGTGGTTCCAACAAAGACCCGGACAAAGCCCACGC | |
| | | CTGCTTATTTACGATGTATCAACTAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCATGACCCC | |
| | | GTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-005 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 80 |
| | | ACCAGCCTCAATTTCATGCAGATCATCACAATCACTTGAGGAAAGCGACG | |
| | | GATACACTTTTCTTGAATGGTTCCAACAAAGACCCGGACAAAGCCCACGC | |
| | | CTGCTTATTTACGATGTATCAGAAAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCTACGACCCC | |
| | | CTCACCTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 81 |
| | Chain | ACCAGCCTCAATTTCATGCAGATCATCACAATCACTTGAGGAAAGCGACG | |
| | | GATACACTTTTCTTGAATGGTTCCAACAAAGACCCGGACAAAGCCCACGC | |
| | | CTGCTTATTTACGATGTATCAGAAAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCTACGACCCC | |
| | | CTCACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-006 | VH | GAAGTACAGTTGGTAGAAAGCGGCGGAGGACTTGTGCAGCCAGGTGGATC | 82 |
| | | TCTCAGACTTTCATGTGCCGCAAGCGGTTTTACTTTTAGTTACTATGGAA | |
| | | TGAACTGGATTAGACAAGCGCCCGGAAAAGGATTGGAATGGATAGGCATG | |
| | | ATATACTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 83 |
| | | ACCAGCCTCAATTTCATGCAGATCATCACAATCACTTGAGGACAGCGACG | |
| | | GAGGAACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGCGACCCACGACCCC | |
| | | CTGACGTTCGGACAAGGAACTAAGCTCGAAATCAAA | |
| | Heavy | GAAGTACAGTTGGTAGAAAGCGGCGGAGGACTTGTGCAGCCAGGTGGATC | 84 |
| | Chain | TCTCAGACTTTCATGTGCCGCAAGCGGTTTTACTTTTAGTTACTATGGAA | |
| | | TGAACTGGATTAGACAAGCGCCCGGAAAAGGATTGGAATGGATAGGCATG | |
| | | ATATACTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 85 |
| | Chain | ACCAGCCTCAATTTCATGCAGATCATCACAATCACTTGAGGACAGCGACG | |
| | | GAGGAACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGCGACCCACGACCCC | |
| | | CTGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-007 | VH | GAAGTACAGTTGGTAGAAAGCGGCGGAGGACTTGTGCAGCCAGGTGGATC | 86 |
| | | TCTCAGACTTTCATGTGCCGCAAGCGGTTTTACTTTTAGTAACTATGGAA | |
| | | TGAACTGGATAAGACAAGCGCCCGGAAAAGGATTGGAATGGATAGGCATG | |
| | | ATATACTACGACAGCTCGGACAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCGGATGTATGGGGTCAAGGAACAATGGTAACCGTGTCAAGC | |
| | VL | GACGTAGTACTGACCCAAAGCCCCCTTTCTCTCCCAGTAACCCTCGGACA | 87 |
| | | ACCAGCCTCAATTTCATGCAGATCATCACAATCACTTGAAGAGAGCGACG | |
| | | GATACACTTTTCTTCATTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | GTGACGTTCGGACAAGGAACTAAGCTCGAAATCAAA | |
| | Heavy | GAAGTACAGTTGGTAGAAAGCGGCGGAGGACTTGTGCAGCCAGGTGGATC | 88 |
| | Chain | TCTCAGACTTTCATGTGCCGCAAGCGGTTTTACTTTTAGTAACTATGGAA | |
| | | TGAACTGGATAAGACAAGCGCCCGGAAAAGGATTGGAATGGATAGGCATG | |
| | | ATATACTACGACAGCTCGGACAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCGGATGTATGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 89 |
| | Chain | ACCAGCCTCAATTTCATGCAGATCATCACAATCACTTGAAGAGAGCGACG | |
| | | GATACACTTTTCTTCATTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | GTGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-008 | VH | GAAGTACAGTTGGTAGAAAGCGGCGGAGGACTTGTGCAACCAGGTGGATC | 90 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 91 |
| | | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAA | |
| | Heavy | GAAGTACAGTTGGTAGAAAGCGGCGGAGGACTTGTGCAACCAGGTGGATC | 92 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 93 |
| | Chain | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-008- | VH | GAAGTACAGTTGGTAGAAAGCGGCGGAGGACTTGTGCAACCAGGTGGATC | 90 |
| QL | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 91 |
| | | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAA | |
| | Heavy | GAAGTACAGTTGGTAGAAAGCGGCGGAGGACTTGTGCAACCAGGTGGATC | 94 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACCAACTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGCTGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 93 |
| | Chain | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-009 | VH | GAAGTACAGTTGGTAGAAAGCGGCGGAGGACTTGTGCAACCAGGTGGATC | 90 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 95 |
| | | ACCAGCCTCAATTTCATGCAGATCATCACAATCACTTGCGGACAGCGACG | |
| | | GAGATACTTTTCTTCACTGGTTCCAACAAAGACCCGGACAAAGCCCACGC | |
| | | CTGCTTATTTACGCGGTATCACACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCATGACCCC | |
| | | GTCACGTTCGGACAAGGAACTAAGCTCGAAATCAAA | |
| | Heavy | GAAGTACAGTTGGTAGAAAGCGGCGGAGGACTTGTGCAACCAGGTGGATC | 92 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 96 |
| | Chain | ACCAGCCTCAATTTCATGCAGATCATCACAATCACTTGCGGACAGCGACG | |
| | | GAGATACTTTTCTTCACTGGTTCCAACAAAGACCCGGACAAAGCCCACGC | |
| | | CTGCTTATTTACGCGGTATCACACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCATGACCCC | |
| | | GTCACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-010 | VH | GAAGTACAGTTGGTAGAAAGCGGCGGAGGACTTGTGCAGCCAGGTGGATC | 97 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCACG | |
| | | ACGCCCGATAAATGGGGTCAAGGAACAATGGTAACCGTGTCAAGC | |
| | VL | GACGTAGTACTGACCCAAAGCCCCCTTTCTCTCCCAGTAACCCTCGGACA | 98 |
| | | ACCAGCCTCAATTTCATGCAGATCATCACAATCACTTGAGGAGAGCGACG | |
| | | GATACACTTTTCTTGAGTGGTTCCAACAAAGACCCGGACAAAGCCCACGC | |
| | | CTGCTTATTTACGAGGTATCACATAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCCACGACCCC | |
| | | CTGACGTTCGGACAAGGAACTAAGCTCGAAATCAAA | |
| | Heavy | GAAGTACAGTTGGTAGAAAGCGGCGGAGGACTTGTGCAGCCAGGTGGATC | 99 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCACG | |
| | | ACGCCCGATAAATGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 100 |
| | Chain | ACCAGCCTCAATTTCATGCAGATCATCACAATCACTTGAGGAGAGCGACG | |
| | | GATACACTTTTCTTGAGTGGTTCCAACAAAGACCCGGACAAAGCCCACGC | |
| | | CTGCTTATTTACGAGGTATCACATAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCCACGACCCC | |
| | | CTGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-011 | VH | GAAGTACAGTTGGTAGAAAGCGGCGGAGGACTTGTGCAGCCAGGTGGATC | 101 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGAATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCTCG | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 102 |
| | | ACCAGCCTCAATTTCATGCAGATCATCACAATCACTTGAGGACAGCGACG | |
| | | GAGGCACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTCAGCTTCGGACAAGGAACTAAGCTCGAAATCAAA | |
| | Heavy | GAAGTACAGTTGGTAGAAAGCGGCGGAGGACTTGTGCAGCCAGGTGGATC | 40 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGAATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCTCG | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 64 |
| | Chain | ACCAGCCTCAATTTCATGCAGATCATCACAATCACTTGAGGACAGCGACG | |
| | | GAGGCACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTCAGCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |

In some embodiments, the anti-HIV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 73.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 75.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 77.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 79.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 81.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 84, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 85.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 88, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 89.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 92, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 93.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 94, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 93.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 92, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 96.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 99, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 100.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 40, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 64.

In some embodiments, the anti-HIV antibodies described herein can be used for delivering a molecular payload to a target cell or a target tissue (e.g., a cell or tissue that expresses HJV). Accordingly, the anti-HJV antibody described herein can be linked to a molecular payload. The complexes described herein may be used in various applications, e.g., diagnostic or therapeutic applications.

In some embodiments, the complex described herein is used to modulate the activity or function of at least one gene, protein, and/or nucleic acid. In some embodiments, the molecular payload is responsible for the modulation of a gene, protein, and/or nucleic acids. A molecular payload may be a small molecule, protein, nucleic acid, oligonucleotide, or any molecular entity capable of modulating the activity or function of a gene, protein, and/or nucleic acid in a cell. In some embodiments, a molecular payload is an oligonucleotide that targets a disease-associated repeat in muscle cells.

### IV. Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The anti-HIV antibody containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to a HEPES (4-(2-hydroxyethyl)-1-piperazineethanesulfonic acid) buffer, Dulbeco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

In some embodiments, the buffer agent in the pharmaceutical composition described herein may maintain a pH value of about 5-8. For example, the pH of the pharmaceutical composition can be about 7.1, 7.2, 7.3, 7.4, 7.5, 7.6, 7.7, 7.8, 7.9, or 8.0. In other examples, the pharmaceutical composition may have a pH value lower than 7, for example, about 7, 6.8, 6.5, 6.3, 6, 5.8, 5.5, 5.3, or 5.

The pharmaceutical composition described herein comprises one or more suitable salts. A salt is an ionic compound that can be formed by the neutralization reaction of an acid and a base. (Skoog, D. A; West, D. M.; Holler, J. F.; Crouch, S. R. (2004). “chapters 14-16”. Fundamentals of Analytical Chemistry (8th ed.)). Salts are composed of related numbers of cations (positively charged ions) and anions (negative ions) so that the product is electrically neutral (without a net charge).

In some embodiments, the pharmaceutical compositions can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). In some embodiments, the pharmaceutical composition can be formulated for intravenous injection. In some embodiments, the pharmaceutical composition can be formulated for subcutaneous injection.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous or subcutaneous solution bag or vial having a stopper pierceable by a hypodermic injection needle.

### V. Combination Therapies

In some embodiments, an immunomodulatory agent or erythropoietin stimulating agent provided herein for the treatment of anemia, e.g., as associated with chronic kidney disease. Such agents include erythropoietin stimulating agents (ESAs). Accordingly, in some embodiments, erythropoietin (EPO) is administered as an additional therapeutic agent in the methods described herein. In some embodiments, the EPO is selected from Epoetin alfa (Epogen/Procrit), Darbepoetin alfa (Aranesp), Methoxy polyethylene glycol-epoetin beta (Mircera), Epoetin alfa-epbx (Retacrit); and biosimilars to Epogen/Procrit.

In some embodiments, any of the disclosed hemojuvelin antagonists may be administered in a combination therapy with a therapeutic agent selected from a growth differentiation factor (GDF) trap, oral iron, IV iron, a HIF-PHI, and a red blood cell transfusion. Exemplary GDF traps include sotatercept and luspatercept.

In some embodiments, the additional therapeutic agent is an IV iron therapy, such as Iron Isomaltoside (MonoFerric®). Additional exemplary IV iron therapies include, but are not limited to, Iron Sucrose (Venofer®), Ferric Carboylmaltose (Ferrinject® or Injectofer®), Ferumoxytol (Ferraheme®), Iron Dextran (Imferon®), and sodium ferric gluconate in iron sucrose solution (Ferrlecit®).

In some embodiments, the additional therapeutic agent is an oral iron therapy. In some embodiments, the hemojuvelin antagonists provided herein may be combined with oral iron therapy to facilitate restoration of iron levels and/or treat anemic subjects who are experiencing intolerance to oral iron or an unsatisfactory response to oral iron. In some embodiments, a combination therapy of anti-hemojuvelin antibody and oral iron is administered to a subject that presents with serum ferritin levels lower than 100 ng/ml and a TSAT lower than 30%. Examples of oral iron therapies include, but are not limited to, ferrous sulfate, ferric maltol (Accrufer®), ferrous gluconate, ferrous succinate, iron polymaltose, polysaccharide-iron complex, and ferrous sulfate. In other cases, iron may be administered intramuscularly (e.g., as iron sorbitol citrate).

In some embodiments, the additional therapeutic agent is an HIF-PHI, such as daprodustat, roxadustat and vadadustat. In some embodiments, the additional therapeutic agent is roxadustat.

Other immunomodulatory agents that may be used in combination therapies of the disclosure include, for example, corticosteroids. In some embodiments, the immunomodulatory agents are advantageous in that they have beneficial effects in reducing inflammation and in promoting erythropoiesis. Danazol, for example, is a steroid compound having hematopoietic stimulatory and immunomodulatory effects. For example, in some embodiments, danazol has antagonistic effects on glucocorticoid receptors, resulting in upregulating effects on erythropoiesis (see, e.g., Chai K Y, et al., Danazol: An Effective and Underutilized Treatment Option in Diamond-Blackfan Anaemia. Case Reports in Hematology. Volume 2019, Article ID 4684156). Other useful immunomodulatory agents include thalidomide and derivatives or analogs thereof, such as lenalidomide, and pomalidomide.

### VI. Methods of Treatment

Aspects of the disclosure relate to methods for treating anemia of kidney disease and/or one or more conditions arising as a result of anemia of kidney disease in a subject. Particular aspects of the disclosure relate to methods for treating anemia of CKD. Additional aspects of the disclosure relate to methods for treating anemia in a subject is identified as having a level of glomerular filtration rate (GFR) of less than 90 mL/min per 1.73 m², less than 60 mL/min per 1.73 m², less than 30 mL/min per 1.73 m², less than 15 mL/min per 1.73 m², or less than 7 mL/min per 1.73 m². In some embodiments, the subject has a GFR level between 15 and 59 ml/min. Additional aspects relate to methods for treating anemia from iron deficiency associated with, or coincident with, CKD.

In some embodiments, methods provided herein are useful for treating subjects having anemia associated with kidney disease so as to decrease hepcidin levels or activity. In some embodiments, the subject may experience an improvement in iron uptake from the gastrointestinal system (i.e., from diet). In some embodiments, the subject may experience a restoration, either partial or complete, of iron levels. In some embodiments, the subject may have had an unsatisfactory response to oral iron treatment. In some embodiments, the subject may have a CKD that is non-dialysis dependent (CKD-NDD). In some embodiments, the subject has a non-hemodialysis dependent chronic kidney disease.

In exemplary embodiments, the subject has a transferrin saturation (TSAT) level less than 50%, less than 40%, less than 30%, or less than 20%. In some embodiments, the subject has been identified as having hemoglobin levels in the range of 1.5 to 2.0 g/dL or 2.0 to 4.0 g/dL or more below normal hemoglobin levels. In some embodiments, the subject presents with a serum hemoglobin level of less than 11 g/dL. 10 g/dL, 9 g/dL, or 8 g/dL. In some embodiments, the subject has serum ferritin levels lower than 300 ng/ml, 200 ng/ml or 100 ng/mL. In some embodiments, the subject presents with serum ferritin levels lower than 100 ng/ml and a TSAT lower than 30%.

In some embodiments, the administration of the hepcidin antagonist (e.g., the anti-HJV antibody) increases hemoglobin (HGB) levels at least 1 g/dL from baseline. In some embodiments, the administration of the hepcidin antagonist increases hemoglobin level in a subject by at least 1 g/dL relative to an untreated subject. In some embodiments, any of the disclosed methods of administration of the hepcidin antagonist results in an increase in hemoglobin levels in a subject at least 2, 4, 6, 8, 10, 11, 12, 13, 14, 15, 16, 17, 18, 19, 20, or more than 20 g/dL relative to an untreated subject. In particular embodiments, any of the disclosed methods result in increased hemoglobin levels of about 17 g/dL (or 170 g/L). In some embodiments, any of the disclosed methods result in increased hemoglobin levels of about 10%, 11%, 12%, 13%, 14%, 15%, 16%, 17%, 18%, 19%, 20% relative to an untreated subject. These increased HGB levels may be observed within 10, 20, 30, 40, or more than 40 days of treatment. In particular, these increased HGB levels may be observed after about 42 days of treatment.

In some embodiments, the administration of the hepcidin antagonist (e.g., the anti-HJV antibody) increases reticulocyte hemoglobin (Ret-HGB) levels in a subject by at least 0.2, 0.3, 0.4, 0.5, 0.6, 0.7, 0.75, 0.8, 0.85, 0.9, 0.95, 1.0, or more than 1.0 pg relative to an untreated subject. Ret-HGB is a measure of cellular hemoglobinization. In particular embodiments, any of the disclosed methods result in increased Ret-HGB levels of about 0.85 pg. In some embodiments, any of the disclosed methods result in increased hemoglobin levels of about 1%, 1.5%, 2%, 2.5%, 3%, 3.5%, 4%, 4.5%, 5%, or more than 5% relative to an untreated subject. These increased Ret-HGB levels may be observed within 10, 20, 30, 40, or more than 40 days of treatment. In particular, these increased Ret-HGB levels may be observed after about 42 days of treatment.

In some aspects, the disclosure relates to compositions and methods for treating CKD in a subject. In some embodiments, a subject to be treated in accordance with the disclosure may be identified based on an appropriate diagnostic methodology, as described, for example, in National Kidney Foundation, K/DOQI clinical practice guidelines for chronic kidney disease: Evaluation, classification, and stratification, Am. J. Kidney Dis. 2002:39 (Suppl 1): S1-S266; Cullis, J O, Diagnosis and management of anaemia of chronic disease: current status British Journal of Haematology, Volume 154, Issue 3, August 2011 pages 289-300; and Madua A J and Ughasoro M D, Anaemia of Chronic Disease: An In-Depth Review Med Princ Pract. 2017 January; 26 (1): 1-9, the contents of each of which are incorporated herein by reference. Typically, diagnosis of anemia involves an evaluation of signs and symptoms of the underlying chronic condition combined with an assessment of indicia of anemia and/or defects in iron metabolism, including, for example, through an analysis of complete blood count (CBC), serum iron, ferritin, transferrin, reticulocyte count, and other markers.

In some embodiments, a subject in need of treatment in accordance with the disclosure may be identified based on a reduced EPO production. For example, under normal physiological conditions, levels of EPO are inversely correlated with hemoglobin levels and tissue oxygenation, but in chronic inflammatory conditions the EPO response is blunted, leading to inadequate levels of EPO for the degree of anemia, and this is thought to be mediated via inflammatory cytokines such as IL-1 and tumor necrosis factor-α (TNF-α). Accordingly, in some embodiments, a blunted EPO response may be diagnostic of anemia of chronic disease (ACD), such as anemia of chronic kidney disease, in a subject.

In some embodiments, a subject in need of treatment in accordance with the disclosure may be identified based on a reduced erythroid responsiveness. For example, anemia may be characterized by a reduced proliferation and differentiation of erythroid progenitor cells. It has been shown that macrophages from patients with anemia suppress colony formation in vitro due to inhibitory effects of inflammatory cytokines (e.g., interferon-γ) on growth of erythroid burst-forming units (BFU-E) and erythroid colony-forming units (CFU-E), and that this effect could be overcome by addition of high concentrations of EPO to the culture systems. It has also been shown that bone marrow cultures from patients with active rheumatoid arthritis showed defective growth when compared to normal controls, and that there was an inverse correlation between colony growth and levels of TNF-α in the culture supernatant. Moreover, these effects were reversed both in vitro and in vivo following treatment with infliximab, an antibody against TNF-α. Accordingly, in some embodiments, a reduced erythroid responsiveness in a subject can be identified using these or similar such assays for evaluating erythroid responsiveness.

In some embodiments, a subject in need of treatment in accordance with the disclosure may be identified based on an anemic state that is mild to moderate and/or normochromic and normocytic (although anemia may become microcytic as disease progresses). In some embodiments, a subject is identified based on a low reticulocyte count. Inflammation in a subject may be inferred from other features of the blood count, such as neutrophilia, monocytosis or thrombocytosis, and through measurement of non-specific inflammatory markers, such as C-reactive protein (CRP) or erythrocyte sedimentation rate (ESR).

In some embodiments, a subject is identified by determining a ratio of serum transferrin receptor (sTFR) to ferritin. The measurement of sTFR, the truncated fragment of the membrane receptor, has been described as an indicator for differentiating between anemia and IDA in a subject. The transferrin receptor is found on virtually all cells in the body, but is present at high levels on erythroid progenitors. sTFR levels increase in IDA as the availability of iron for erythropoiesis decreases, whereas in anemia levels may not differ from steady state because transferrin receptor expression is negatively affected by inflammatory cytokines. The ratio of sTFR to the log of the serum ferritin may be used in the diagnosis of anemia in a subject, and in some cases may be used for differentiating anemia from IDA. A ratio of less than 1 makes anemia likely, whereas ratios of greater than 2 suggest that iron stores are deficient, with or without anemia.

In some embodiments, a subject in need of treatment in accordance with the disclosure may be identified using red cell indices. For example, the reticulocyte hemoglobin content (CHr) and the percentage hypochromic red cells (% HYPO) can provide information about iron supply to the erythron, and may be useful in guiding the management of anemia (e.g., ACD (e.g., anemic of chronic kidney disease)). CHr is a measure of hemoglobin in the most recently formed erythrocytes, while the % HYPO indicates the percentage of cells with hemoglobin content of <280 g/l. The former gives a relatively acute evaluation of recent bone marrow activity (e.g., 48 hours), whereas the latter gives a time-averaged picture (e.g., 20-120 days). Similar indices can be reported by the Sysmex XE-2100 analyser (Sysmex, Mundelein, IL, USA), which derives RET-Y (equivalent to CHr) and RBC-Y (equivalent to HYPO %). CHr has been shown to be a useful tool in the detection of early iron deficiency, as well as in monitoring early response to iron therapy.

In some embodiments, a subject has previously received an erythropoietin stimulating agent. In some embodiments, the erythropoietin stimulating agent is selected from the group consisting of danazol, prednisone, thalidomide, lenalidomide, and pomalidomide.

Determination of whether an amount of the HJV antagonist achieved the therapeutic effect would be evident to one of skill in the art based on the teachings provided herein. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. The particular dosage regimen, i.e., dose, timing and repetition, used in the method described herein will depend on the particular subject and that subject's medical history, as discussed herein.

Empirical considerations, such as time to maximum effect, half-life, and/or time above a specific concentration generally will contribute to the determination of the dosage.

In some embodiments, dosages for a HJV antagonist as described herein may be determined empirically in individuals who have been given one or more administration(s) of the antibody. Individuals are given incremental dosages of the antagonist. To assess efficacy of the antagonist, an indicator of the disease/disorder can be followed.

Dosing frequencies may vary in accordance with the claimed methods. In some embodiments, a composition will be administered once. In some embodiments, a treatment will be administered on multiple occasions. In some embodiments, dosing frequency is every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. In some embodiments, a composition will be administered daily, biweekly, weekly, bimonthly, monthly, semi-monthly, quarterly, or at any time interval that provide suitable (e.g., maximal) efficacy while minimizing safety risks to the subject. In some embodiments, dosing frequency is over a period of about 45 days, 50 days, 55, days, 60 days, or 65 days. In some embodiments, the dosing period is between 40 and 60 days, 40 and 55 days, 50 and 60 days, or 50 and 55 days. In some embodiments, the dosing period is between 50 and 55 days. Generally, the efficacy and the treatment and safety risks may be monitored throughout the course of treatment. In some embodiments, the hemojuvelin antagonist is administered once monthly. In some embodiments, the hemojuvelin antagonist is administered once quarterly.

In some embodiments, administration of HJV antagonist results in a decrease in serum hepcidin-25 concentration and/or increase serum TSAT %, and in some embodiments, these effects persist for a period of time (e.g., one month or more). Accordingly, in some embodiments, timing and frequency of administration of HJV antagonist can be determined by monitoring one or more biomarkers, e.g., criteria to assess iron availability or flag possible iron overload. For example, in some embodiments, HJV antagonist is administered intermittently or in accordance with the level of a particular biomarker such as serum hepcidin-25 levels or transferrin saturation percentage (TSAT %). In some embodiments, a biomarker level described herein can be used to determine whether a subject is a candidate for treatment. However, in some embodiments, a biomarker may be used to determine whether to continue treatment or to resume a treatment or to halt a treatment, e.g., with a HJV antagonist.

In some embodiments, administration of HJV antagonist results in a decrease in urine hepcidin concentration and/or an increase in serum TSAT %.

For example, in some embodiments, a subject may be considered as not being a candidate for treatment if TSAT % of the subject is at or above 70%, at or above 75%, at or above 80%, at or above 85%, at or above 90%, or at or above 95%. In some cases, if TSAT % of the subject is at or above 70%, at or above 75%, at or above 80%, at or above 85%, at or above 90%, or at or above 95%, an ongoing treatment with a HJV antagonist may be stopped or temporarily stopped, e.g., to prevent iron overload. In other embodiments, administration of an anti-HJV antibody may be performed when a TSAT % of a subject is at or below 95%, at or below 90%, at or below 80%, at or below 70%, at or below 65%, at or below 60%, at or below 55%, at or below 50%, at or below 45%, at or below 40%, at or below 35%, or at or below 30%. Thus, in some embodiments, TSAT % of a subject can be monitored, e.g., continuously or periodically, while a patient is receiving a treatment or under care of a treating physician, e.g., for anemia, to prevent iron overload or otherwise to assess whether further treatments are appropriate. It should be appreciated, however, that other suitable markers may be monitored to determine dosage and dosage frequency (including, for example, ferritin levels, serum iron levels, creatinine levels, etc.) in accordance with the methods provided herein.

In some embodiments, a subject may be administered a composition provided herein (e.g., HJV antagonist) at one or more intervals during a set period of time. In some cases, periods of time during which a subject is administered a composition at one or more intervals may be separated by periods of time in which the subject is not administered the composition. In some embodiments, the relative durations of respective periods of time may depend on the subject's response to treatment or severity of disease or both and/or may be determined based on the judgment of a treating physician. For example, in some embodiments, during the course of a year a subject may be administered a composition weekly, biweekly, monthly or semi-monthly for two months, and optionally then the administration is stopped for ten months. In some embodiments, during the course of a year a subject may be administered a composition weekly, biweekly, monthly or semi-monthly for three months, and optionally then the administration is stopped for nine months. In some embodiments, during the course of a year a subject may be administered a composition weekly, biweekly, monthly or semi-monthly for four months, and optionally then the administration is stopped for eight months. In some embodiments, during the course of a year a subject may be administered a composition weekly, biweekly, monthly or semi-monthly for five months, and optionally then the administration is stopped for seven months. In some embodiments, during the course of a year a subject may be administered a composition weekly, biweekly, monthly or semi-monthly for six months and then the administration is stopped for six months. In some embodiments, during the course of a year a subject may be administered a composition weekly, biweekly, monthly or semi-monthly for seven months and then the administration is stopped for five months. In some embodiments, during the course of a year a subject may be administered a composition weekly, biweekly, monthly or semi-monthly for eight months and then the administration is stopped for four months. In some embodiments, during the course of a year a subject may be administered a composition weekly, biweekly, monthly or semi-monthly for nine months and then the administration is stopped for three months. In some embodiments, during the course of a year a subject may be administered a composition weekly, biweekly, monthly or semi-monthly for ten months and then the administration is stopped for two months. In some embodiments, during the course of a year a subject may be administered a composition weekly, biweekly, monthly or semi-monthly for two months on, two months off; or for three months on, three months off; or for four months on, four months off. In some embodiments, during the course of a year, a subject may be administered a composition quarterly for the entire duration of the year (i.e., four times). In some embodiments, during the course of a year, a subject may be administered a composition quarterly twice, or three times, but not four times.

Generally, for administration of any of the antibodies described herein, a dose may be about 0.01 mg/kg. 0.05 mg/kg. 0.1 mg/kg, 0.2 mg/kg, 0.3 mg/kg, 0.4 mg/kg, 0.5 mg/kg, 0.6 mg/kg, 0.8 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg or 100 mg/kg.

In some embodiments, the dosage the anti-HJV antibody is up to 0.01 mg/kg, up to 0.05 mg/kg, up to 0.1 mg/kg, up to 0.2 mg/kg, up to 0.3 mg/kg, up to 0.4 mg/kg, up to 0.5 mg/kg, up to 0.6 mg/kg, up to 0.8 mg/kg, up to 1 mg/kg, mg/kg, up to 2 mg/kg, up to 3 mg/kg, up to 4 mg/kg, up to 5 g/kg, up to 6 mg/kg, up to 7 mg/kg, up to 8 mg/kg, up to 9 mg/kg, up to 10 mg/kg, up to 20 mg/kg, up to 30 mg/kg, up to 40 mg/kg, up to 50 mg/kg, up to 60 mg/kg, up to 70 mg/kg, 80 mg/kg, up to 90 mg/kg, up to 100 mg/kg or more.

However, in some embodiments, the dose of the anti-HJV antibody can be in a range of 0.01 mg/kg to 100 mg/kg, 0.01 mg/kg to 10 mg/kg, 0.5 mg/kg to 15 mg/kg, 0.6 mg/kg to 15 mg/kg. 0.8 mg/kg to 15 mg/kg, 1 mg/kg to 15 mg/kg, 5 mg/kg to 25 mg/kg, 10 mg/kg to 30 mg/kg, 20 mg/kg to 40 mg/kg, 30 mg/kg to 50 mg/kg, 40 mg/kg to 60 mg/kg, 50 mg/kg to 75 mg/kg, or 50 mg/kg to 100 mg/kg.

In some embodiments, the antibodies described herein are administered to a subject in need of the treatment at an amount sufficient to inhibit the activity of the target antigen (e.g., an amount sufficient to inhibit HJV-induced BMP signaling) by at least 20% (e.g., 30%, 40%, 50%, 60%, 70%, 80%, 90% or greater) in vivo. In other embodiments, the antibody is administered in an amount effective in reducing the activity level of a target antigen by at least 20% (e.g., 30%, 40%, 50%, 60%, 70%, 80%, 90% or greater).

In some embodiments, an antibody can be administered parenterally. For example, a parenterally administered composition may be administered by subcutaneous, intracutaneous, intravenous, intraperitoneal, intratumor, intramuscular, intraarticular, intraarterial, or infusion techniques. In addition, it can be administered to the subject via injectable depot routes of administration such as using 1-, 3-, or 6-month depot injectable or biodegradable materials and methods.

In some embodiments, an antibody (e.g., an anti-HJV antibody) is administered intravenously. In some embodiments, an antibody (e.g., an anti-HJV antibody) is administered subcutaneously. In some embodiments, subcutaneous administration of an anti-HJV antibody results in similar bioavailability compared to intravenous administration of the same antibody at the same dose.

In some embodiments, subcutaneous administration of the anti-HIV antibody yields comparable pharmacodynamics effects (e.g., decreased circulating hepcidin-25 levels, increased TSAT %, and/or increased serum iron levels) at lower maximum concentrations (Cₘₐₓ) of the anti-HJV antibody compared to intravenous administration of the same antibody. Cₘₐₓ is the maximum (or peak) serum concentration that a drug (e.g., an anti-HJV antibody) after the drug has been administered and before the administration of a second dose. In some embodiments, achieving a low Cₘₐₓ within a short period of time (e.g., within 12 hours, within 24 hours, etc) after administration of an anti-HJV antibody minimizes undesirable increases in serum iron response, and/or minimizes chances of off-target effects of the antibody (e.g., binding to RGMa). In some embodiments, blunting Cₘₐₓ by subcutaneous administration of an anti-HJV antibody avoids an undesirably sharp increase in serum iron response. In some embodiments, blunting Cₘₐₓ by subcutaneous administration of an anti-HJV antibody reduces the off-target effects of the antibody. In some embodiments, the Cₘₐₓ reached by subcutaneous administration is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than the Cₘₐₓ reached by intravenous administration of an anti-HJV antibody.

In some embodiments, any of the disclosed methods (such as any of the disclosed methods of subcutaneous administration) result in increased serum iron levels in the subject, relative to an untreated subject, of about 25, 27.5 30, 32.5, 35, 38.5, 40, or 45 μmol/L. In particular embodiments, any of the disclosed methods result in increased serum iron levels of about 35 μmol/L. In some embodiments, any of the disclosed methods result in increased serum iron levels of about 95%, 97.5%, 100%, 102.5%, 105%, 110%, 115%, or 120% relative to an untreated subject. In particular embodiments, any of the disclosed methods result in increased serum iron levels of about 100-110%, and in particular about 109% or 119% on average. These increased serum levels may be observed within 10, 20, 30, 40, or more than 40 days of treatment. In particular, these increased serum levels may be observed after about 42 days of treatment.

In some embodiments, any of the disclosed methods (such as any of the disclosed methods of subcutaneous administration) result in increased red blood cell (RBC) counts (e.g., counts of mature RBCs) in the subject, relative to an untreated subject, of about 1, 2, 2.5, 3, 4, 5, 6, 7, 7.5, 8, 9, or 10×10⁵ cells/μL. In particular embodiments, any of the disclosed methods result in increased RBC counts of about 4 or 5×10⁵ cells/μL. In some embodiments, any of the disclosed methods result in increased RBC counts of about 5%, 6%, 6.5%, 7%, 7.25%, 7.5%, 7.75%, 8%, 8.5%, 9%, 9.25%, 9.5%, 10%, or more than 10% relative to an untreated subject. In particular embodiments, any of the disclosed methods result in increased RBC counts of greater than about 7% (such as 7.25%). In some embodiments, RBC counts are raised by between 7% and 9.5%. These increased RBC counts may be observed within 10, 20, 30, 40, or more than 40 days of treatment. In particular, these RBC counts may be observed after about 42 days of treatment.

In some embodiments, any of the disclosed methods (such as any of the disclosed methods of subcutaneous administration) result in decreased reticulocyte (Ret) counts in the subject, relative to an untreated subject, of about 90, 100, 100, 120 or 125×10⁹ cells/L. In particular embodiments, any of the disclosed methods result in decreased Ret counts of about 100×10⁹ cells/μL. In some embodiments, any of the disclosed methods result in decreased Ret counts of about 45%, 50%, or 55% relative to an untreated subject. These decreased Ret counts may be observed within 10, 20, 30, 40, or more than 40 days of treatment. In particular, these Ret counts may be observed after about 42 days of treatment.

For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline. Ringer's solution or other suitable excipients. Other injectable compositions may contain various carriers such as vegetable oils, dimethyllactamide, dimethyformamide, ethyl lactate, ethyl carbonate, isopropyl myristate, ethanol, and polyols (glycerol, propylene glycol, liquid polyethylene glycol, and the like). In some cases, preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. Sec, e.g., PCT Publication No. WO 2000/53211 and U.S. Pat. No. 5,981,568.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents. Treatment efficacy for a target disease/disorder can be assessed by methods well-known in the art.

The anti-HJV antibody and treatment methods involving such as described in the present disclosure may be utilized in combination with other types of therapy for the target disease or disorder disclosed herein. In this context, an antibody composition and a therapeutic agent may be given either simultaneously or sequentially. Examples include chemotherapy, immune therapy (e.g. therapies involving other HJV antagonists), surgery, radiation, gene therapy, and so forth, or anti-infection therapy. Such therapies can be administered simultaneously or sequentially (in any order) with the treatment according to the present disclosure.

For example, the combination therapy can include the anti-HIV antibody and pharmaceutical composition described herein, co-formulated with and/or co-administered with, at least one additional therapeutic agent described herein. Such combination therapies may advantageously utilize lower dosages of the administered therapeutic agents, thus preventing possible toxicities or complications associated with the various monotherapies. Moreover, the additional therapeutic agents disclosed herein may act on pathways in addition to or distinct from the hepcidin/BMP pathway, and thus may enhance and/or synergize with the effects of the anti-HJV antibodies.

In some embodiments, a subject has previously received an erythropoietin stimulating agent. In some embodiments, the erythropoietin stimulating agent is selected from the group consisting of danazol, prednisone, thalidomide, lenalidomide, and pomalidomide.

In some embodiments, a subject has previously received a JAK-STAT pathway inhibitor. In some embodiments, the JAK-STAT pathway inhibitor is a JAK inhibitor or a STAT inhibitor. In some embodiments, the JAK inhibitor is selective for one or both of subtypes JAK1 and JAK2 (e.g., a JAK1/2 inhibitor). In some embodiments, the STAT inhibitor is a STAT3 inhibitor. In some embodiments, the JAK1/2 or STAT3 inhibitor is selected from the group consisting of ruxolitinib, fedratinib, momelotinib, pacritinib, INCB039110, AG490, and PpYLKTK (where “pY” represents a phosphorylated tyrosine (Y) residue (PY*LKTK); SEQ ID NO: 131).

In some embodiments, a subject has previously received a growth factor ligand trap. In some embodiments, the growth factor ligand trap is a transforming growth factor beta (TGF-β) ligand trap. In some embodiments, the TGF-β ligand trap is a GDF trap such as sotatercept or luspatercept. See U.S. Pat. No. 8,216,997, the contents of each of which are herein incorporated by reference. In some embodiments, a subject has previously received an anti-fibrotic agent. In some embodiments, the anti-fibrotic agent is PRM-151.

In some embodiments, a subject in need of treatment in accordance with the disclosure continues to receive a therapeutic treatment for a hematologic disorder. The disclosure therefore provides, in some aspects, compositions and methods for treating anemia (e.g., ACD (e.g., anemic of chronic kidney disease)) and/or one or more conditions arising as a result of the anemia by administering to a subject in need thereof an anti-HJV antibody in combination with one or more therapeutic treatments for a hematologic disorder.

In some embodiments, the anemia is characterized based on Reticulocyte Hemoglobin Content (RET-He or CHr). Reticulocyte hemoglobin content measures the amount of hemoglobin in reticulocytes. A normal range of CHr is about 28 to 36 pg/cell. In some embodiments, the subject has a CHr lower than the normal range. In some embodiments, the subject has a CHr less than 36 pg/cell, less than 35 pg/cell, less than 34 pg/cell, less than 33 pg/cell. less than 32 pg/cell, 31 pg/cell, 30 pg/cell, 29 pg/cell, less than 28 pg/cell, less than 27 pg/cell, less than 26 pg/cell, less than 25 pg/cell, less than 24 pg/cell, less than 23 pg/cell, less than 21 pg/cell, or less than 20 pg/cell. It should be appreciated, however, that other suitable markers (e.g., TSAT %, serum iron levels, total iron binding capacity (TIBC), ferritin levels, hemoglobin levels, hepatic iron content, hepcidin levels, IL-6 levels, creatinine levels, etc) may be evaluated to determine if the subject is suitable for method of treatment described herein.

In some embodiments, the anemia is characterized by hepatic iron levels. In some embodiments, a normal range of hepatic iron level is 200-2,400 μg/g dry weight in males and 400-1,600 μg/g dry weight in female. In some embodiments, the subject has a higher than normal of hepatic iron level. In some embodiments, the patient has hepatic iron levels higher than 1000 μg/g dry weight (e.g., between about 1000 μg/g to 1200 μg/g dry weight, between about 1000 μg/g to 1500 μg/g dry weight, or between about 1200 μg/g to 1500 μg/g dry weight), higher than 1500 μg/g dry weight (e.g., between about 1500 μg/g to 1800 μg/g dry weight, between about 1500 μg/g to 2000 μg/g dry weight, or between about 1800 μg/g to 2000 μg/g dry weight), higher than 2000 μg/g dry weight (e.g., between about 2000 μg/g to 2200 μg/g dry weight, between about 2000 μg/g to 2500 μg/g dry weight, or between about 2200 μg/g to 2500 μg/g dry weight), higher than 2500 μg/g dry weight (e.g., between about 2500 μg/g to 2800 μg/g dry weight, between about 2500 μg/g to 3000 μg/g dry weight, or between about 2800 μg/g to 3000 μg/g dry weight), or higher than 3000 μg/g dry weight.

In some embodiments, the anemia is characterized by low Total Iron Binding Capacity (TIBC). In some embodiments, normal range of TIBC is 250-400 μg/dL. In some embodiments, the subject has a lower than normal TIBC. In some embodiments, the subject has a TIBC of less than 400 μg/dL, less than 350 μg/dL, less than 300 μg/dL, less than 250 μg/dL, less than 200 μg/dL, less than 150 μg/dL, less than 100 μg/dL, less than 90 μg/dL, less than 80 μg/dL, less than 70 μg/dL, less than 60 μg/dL, less than 50 μg/dL, less than 40 μg/dL, less than 30 μg/dL less than 20 μg/dL, or less than 10 μg/dL.

In some embodiments, a subject is administered a hemojuvelin antagonist (e.g., anti-HJV antibodies and compositions thereof) in combination with an erythropoietin stimulating agent. In some embodiments, the erythropoietin stimulating agent is EPO. In some embodiments, the erythropoietin stimulating agent is selected from an immunomodulatory agenda selected from the group consisting of danazol, prednisone, thalidomide, lenalidomide, and pomalidomide.

In some embodiments, a subject is administered a hemojuvelin antagonist (e.g., anti-HJV antibodies and compositions thereof) in combination with a JAK-STAT pathway inhibitor. In some embodiments, the JAK-STAT pathway inhibitor is a JAK inhibitor or a STAT inhibitor. In some embodiments, the JAK inhibitor is selective for one or both of subtypes JAK1 and JAK2 (e.g., a JAK1/2 inhibitor). In some embodiments, the STAT inhibitor is a STAT3 inhibitor. In some embodiments, the JAK1/2 or STAT3 inhibitor is selected from the group consisting of ruxolitinib, fedratinib, momelotinib, pacritinib, INCB039110, AG490, and PpYLKTK (SEQ ID NO: 131). In some embodiments, a subject is administered a hemojuvelin antagonist (e.g., anti-HJV antibodies and compositions thereof) in combination with ruxolitinib.

In some embodiments, the hemojuvelin antagonist (e.g., anti-HJV antibodies and compositions thereof) reduces the extent to which a subject exhibits an anemic response to the JAK-STAT pathway inhibitor. For example, in some embodiments, a subject treated with a JAK-STAT pathway inhibitor as a monotherapy may be characterized as having a deficiency in the ability of blood to transport oxygen as compared to the subject's pretreatment state, a deficiency in red blood cells as compared to the subject's pretreatment state, a deficiency in hemoglobin as compared to the subject's pretreatment state, an/or a deficiency in total blood volume as compared to the subject's pretreatment state. Accordingly, in some embodiments, the hemojuvelin antagonist (e.g., anti-HIV antibody) reduces the extent to which a subject exhibits an anemic response to a JAK-STAT pathway inhibitor selected from the group consisting of ruxolitinib, fedratinib, momelotinib, pacritinib, INCB039110, AG490, and PpYLKTK (SEQ ID NO: 131). In some embodiments, the hemojuvelin antagonist (e.g., anti-HJV antibodies and compositions thereof) reduces the extent to which a subject exhibits an anemic response to ruxolitinib administration.

In some embodiments, a subject is administered a hemojuvelin antagonist (e.g., anti-HJV antibodies and compositions thereof) in combination with a growth factor ligand trap. In some embodiments, the growth factor ligand trap is a transforming growth factor beta (TGF-β) ligand trap. In some embodiments, the TGF-β ligand trap is sotatercept or luspatercept. In some embodiments, a subject is administered a hemojuvelin antagonist (e.g., anti-HJV antibodies and compositions thereof) in combination with an anti-fibrotic agent. In some embodiments, the anti-fibrotic agent is PRM-151.

Successful treatment of a subject in accordance with the disclosure may be determined by methods known in the art or by a skilled practitioner. In some embodiments, HJV antagonist (e.g., anti-HJV antibodies and compositions thereof) treatment is evaluated based on hepcidin (e.g., circulating hepcidin-25 levels) levels in a subject. For example, in some embodiments, baseline hepcidin (e.g., circulating hepcidin-25 levels) levels in a subject are determined (e.g., before treatment with a HJV antagonist (e.g., anti-HJV antibodies and compositions thereof) or otherwise in absence of HJV antagonist (e.g., anti-HJV antibodies and compositions thereof) treatment at the time of determining) and compared to post-treatment hepcidin (e.g., circulating hepcidin-25 levels) levels in the subject. In some embodiments, a subject is successfully treated where a HJV antagonist (e.g., anti-HJV antibodies and compositions thereof) decreases hepcidin (e.g., circulating hepcidin-25 levels) levels in the subject by between about 1 ng/ml and about 300 ng/mL. In some embodiments, the HJV antagonist (e.g., anti-HJV antibodies and compositions thereof) decreases hepcidin (e.g., circulating hepcidin-25 levels) levels in a subject by between about 1 ng/ml and about 200 ng/mL, between about 1 ng/mL and about 100 ng/mL, between about 1 ng/ml and about 50 ng/ml, between about 1 ng/mL and about 10 ng/mL, between about 10 ng/ml and about 100 ng/mL, or between about 10 ng/ml and about 50 ng/mL.

In some embodiments, the present disclosure provides a method for reducing hepcidin (e.g., circulating hepcidin-25 levels) in a subject having anemia (e.g., ACD (e.g., anemia of chronic kidney disease)). In some embodiments, the disclosed methods of administration reduce hepcidin-25 within 4 hours, 6 hours, 8 hours, 12 hours, 28 hours, 24 hours, 2 days, 3 days, 4 days, 5 days, 6 days, 1 week, or two weeks of administration. In some embodiments, the disclosed methods of administration reduce hepcidin (e.g., circulating hepcidin-25 levels) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 99%, or 100% of hepcidin compared to the hepcidin (e.g., circulating hepcidin-25 levels) level in the subject prior to administration. In some embodiments, the disclosed methods of administration reduce hepcidin levels by about 96%.

In some embodiments, the present disclosure provides methods for reducing hepcidin expression (e.g., Hamp gene expression) in the liver of a subject having anemia (e.g., ACD (e.g., anemia of chronic kidney disease)). In some embodiments, the disclosed methods of administration reduce Hamp mRNA expression within 4 hours, 6 hours, 8 hours, 12 hours, 28 hours, 24 hours, 2 days, 3 days, 4 days, 5 days, 6 days, 1 week, or two weeks of administration. In some embodiments, the disclosed methods of administration reduce Hamp mRNA expression within 7, 10, 14, 20-21, 24, 28, 30, 35, 40, or 42 days of administration. In some embodiments, the disclosed methods of administration reduce Hamp mRNA expression by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 99%, or 100% of hepcidin compared to expression levels in the subject prior to administration. In some embodiments, the disclosed methods of administration reduce Hamp expression levels in the subject's liver by about 96%.

In some embodiments, HJV antagonist (e.g., anti-HJV antibodies and compositions thereof) treatment is evaluated based on serum ferritin levels in a subject. For example, in some embodiments, baseline serum ferritin levels in a subject are determined (e.g., before treatment with a HJV antagonist (e.g., anti-HJV antibodies and compositions thereof) or otherwise in absence of HJV antagonist (e.g., anti-HJV antibodies and compositions thereof) treatment at the time of determining) and compared to post-treatment serum ferritin levels in the subject.

In some embodiments, a subject is successfully treated where a HJV antagonist (e.g., anti-HJV antibodies and compositions thereof) decreases serum ferritin levels in the subject by between about 1 ng/mL and about 200 ng/ml. In some embodiments, the HJV antagonist (e.g., anti-HJV antibodies and compositions thereof) decreases serum ferritin levels in a subject by between about 1 ng/mL and about 100 ng/ml, between about 1 ng/ml and about 50 ng/ml, between about 1 ng/mL and about 25 ng/mL, between about 1 ng/ml and about 10 ng/mL, between about 10 ng/mL and about 100 ng/ml, or between about 10 ng/mL and about 50 ng/mL.

In some embodiments, HJV antagonist (e.g., anti-HJV antibodies and compositions thereof) treatment is evaluated based on serum hemoglobin levels in a subject. For example, in some embodiments, baseline serum hemoglobin levels in a subject are determined (e.g., before treatment with a HJV antagonist (e.g., anti-HJV antibodies and compositions thereof) or otherwise in absence of HJV antagonist (e.g., anti-HJV antibodies and compositions thereof) treatment at the time of determining) and compared to post-treatment serum hemoglobin levels in the subject.

In some embodiments, a subject is successfully treated where a HJV antagonist (e.g., anti-HJV antibodies and compositions thereof) increases serum hemoglobin levels in the subject by between about 0.01 g/dL and about 5 g/dL.

In some embodiments, the HJV antagonist (e.g., anti-HJV antibodies and compositions thereof) decreases serum ferritin levels in a subject by between about 0.01 g/dL and about 1 g/dL, between about 0.1 g/dL and about 5 g/dL, between about 1 g/dL and about 5 g/dL, between about 0.01 g/dL and about 0.1 g/dL, between about 0.5 g/dL and about 2.5 g/dL, or between about 0.1 g/dL and about 1 g/dL.

### VII. Comorbidities and Risk Factors

The disclosed methods may be particularly suitable for CKD patients who exhibit a comorbidity of CKD. In particular, these methods may be useful for treating an anemia of CKD in a subject having any of the following comorbidities: diabetes, hypertension, autoimmune disease (such as lupus or another autoimmune condition), complications due to certain medications such as non-steroidal anti-inflammatory drugs (NSAIDs), or congenital heart failure. These and other comorbidities may be a major consideration for physicians when advising patients for or against dialysis.

These methods may be useful for treating an anemia of CKD in a subject having certain other comorbidities or risk factors, such as a birth defect, fatigue, nausea, insomnia, anorexia, or a high BMI; or where the kidney damage is associated with polycystic kidney disease or a glomerular disease, such as acute glomerulonephritis. Several rare diseases and birth defects are coincident with CKD including: Diabetes (such as Type II diabetes), bacterial endocarditis, granulomatosis with polyangiitis, hepatitis B and C, human immunodeficiency virus (HIV) infection, hyperkalemia, light-chain deposition disease, mixed connective tissue disease, mixed cryoglobulinemia, neoplasia, non-alcoholic fatty liver disease (NAFLD), parasitic infection, reflux nephropathy, rheumatoid arthritis, scleroderma, Shiga-toxin- or Streptococcus pneumoniae-related HUS, some cancers, systemic lupus erythematosus, syphilis, thrombotic thrombocytopenia purpura (TTP). Further, the usage of prescription drugs captopril and penicillamine (a chelating agent) has been identified as risk factor for CKD. Recreational use of heroin is another risk factor for CKD. See Zipfel P F, et al. Front Immunol. 2019; 10:2166; and Alwahaibi N Y, et al. Saudi J Kidney Dis Transpl. 2018 November-December; 29 (6): 1256-1266, each of which are herein incorporated by reference.

Accordingly, provided herein are methods of treating anemia of a chronic kidney disease that is associated with one or more of the following comorbidities: diabetes, hypertension, an autoimmune disease, such as lupus, congenital heart failure, complications related to administration of one or more NSAIDs (such as Aleve, Tylenol, Advil or Motrin), a rare disease, and a birth defect.

Further provided herein are methods of treating anemia of a chronic kidney disease that is associated with polycystic kidney disease. Further provided herein are methods of treating anemia of a chronic kidney disease that is associated with a primary or secondary human glomerular disease, such as acute glomerulonephritis, Alport syndrome, amyloidosis (Amyl of hereditary), anti-GBM (Goodpasture's) disease, anti-neutrophil cytoplasmic antibody mediated vasculitis (ANCA), atypical hemolytic uremic syndrome (aHUS), C3 glomerulopathy, chronic transplant mediated glomerulopathy, diabetic nephropathy (DN), Focal and segmental glomerulosclerosis (FSGS), glomerulosclerosis, Henoch-Schönlein purpura (HSP), hypertension (HTN), IgA nephropathy (IgAN), immune complex membranoproliferative glomerulonephritis, renal ischemic reperfusion injury, lupus nephritis (LN), membranous nephropathy, membranoproliferative glomerulonephritis (MPGN), mesangioproliferative glomerulonephritis (MesGN), membranous glomerulopathy (MG), minimal change disease (MCD), post-streptococcal glomerulonephritis (PSGN), Rapidly progressive (crescentic) glomerulonephritis (RPGN).

These methods may be useful for subjects who have previously been administered medications to treat certain conditions, such as diabetes. In some embodiments, provided herein are methods for treating anemia in a subject having CKD that has been previously administered, or is currently administered, a Type II diabetes therapy. Further provided are methods for treating anemia in a subject having CKD that has been previously administered, or is currently administered, a hypertension medication such as an ACE inhibitor.

In some embodiments of the disclosed methods, the subject has not previously undergone a nephrectomy or a kidney transplantation.

### EXAMPLES

### Example 1: Anti-HJV Antibodies Generation and Characterization

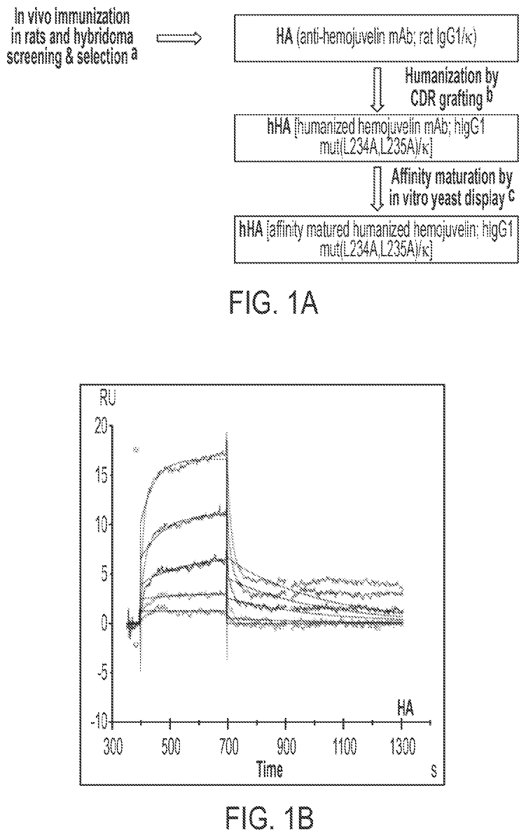

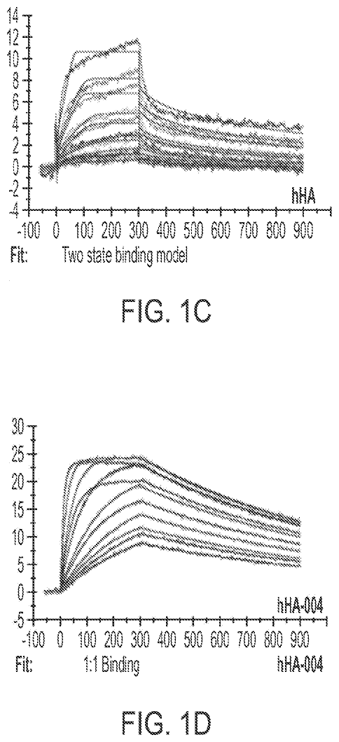

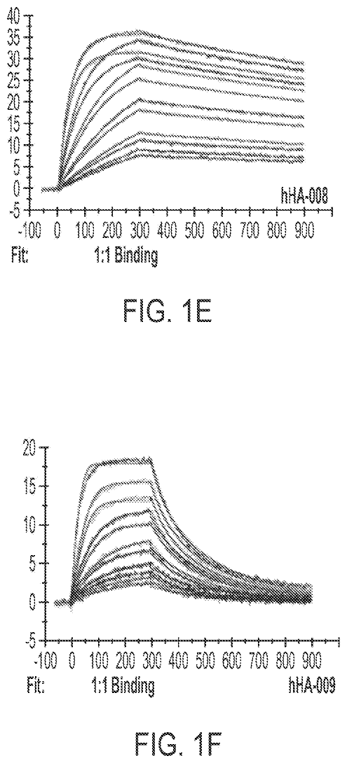

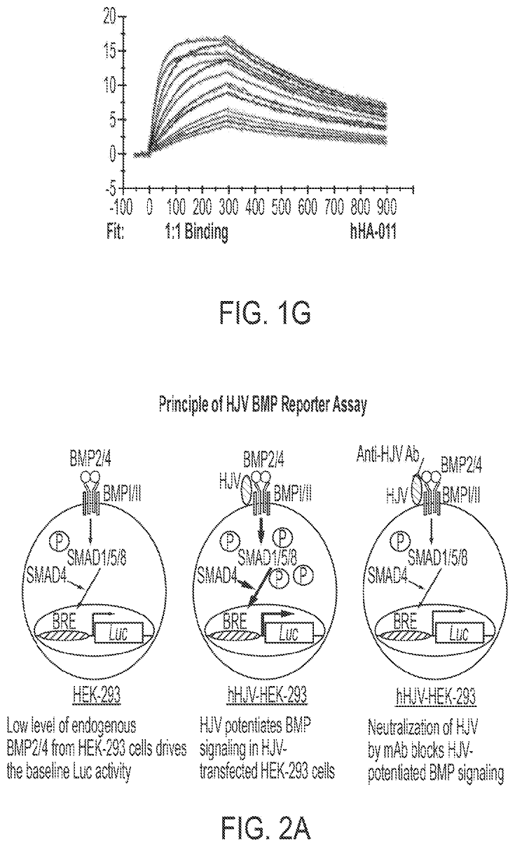

From rats immunized with human hemojuvelin, hybridoma clones capable of binding to human hemojuvelin were identified. The anti-hemojuvelin monoclonal antibodies (mAb with rat IgG1/κ) were humanized by CDR grafting (hHA antibodies with hIgG1 constant region carrying the L234A, L235A mutations). Affinity matured anti-HJV (e.g., HA-001 to HA-011) in vitro yeast display assay. A general process for generation of humanized affinity matured anti-HJV antibodies is illustrated in FIG. 1A.

Binding affinities of the anti-HJV antibodies to soluble human RGMa, Rat RGMa and human RGMc were measured by BIAcore analysis. Table 14 shows the affinity of the anti-hemojuvelin antibodies to human RGMa. Table 15 shows the affinity of the anti-hemojuvelin antibodies to rat RGMa. Table 16 shows the affinity of the anti-hemojuvelin antibodies to human RGMc.

TABLE 14
Binding Affinity (by BIAcore) of
anti-HJV antibodies to Human RGMa

| Ab Name | Kₐ (1/MS) | K_{d} (1/s) | KD (M) |
|---|---|---|---|
| hHA-001 | 1.8E+06 | 6.7E−05 | 3.8E−11 |
| hHA-002 | 1.4E+06 | 5.0E−05 | 3.7E−11 |
| hHA-003 | 1.1E+06 | 1.4E−04 | 1.2E−10 |
| hHA-004 | 1.4E+06 | 2.2E−05 | 1.5E−11 |
| hHA-005 | 1.9E+06 | 2.5E−04 | 1.3E−10 |
| hHA-006 | 1.2E+06 | 1.4E−04 | 1.2E−10 |
| hHA-007 | 5.9E+05 | 8.9E−05 | 1.5E−10 |
| hHA-008 | 8.7E+05 | 2.7E−05 | 3.1E−11 |
| hHA-009 | 8.6E+05 | 4.0E−04 | 4.7E−10 |
| hHA-010 | 8.5E+05 | 1.0E−04 | 1.2E−10 |
| hHA-011 | 9.3E+05 | 9.9E−05 | 1.1E−10 |
| hHA | 6.0E+06 | 2.2E−03 | 3.7E−09 |
| HA | 1.2E+06 | 2.9E−03 | 2.4E−09 |

TABLE 15
Binding Affinity (by BIAcore) of anti-HJV antibodies to rat RGMa

| Ab Name | Kₐ (1/MS) | K_{d} (1/s) | KD (M) |
|---|---|---|---|
| hHA-001 | 1.2E+06 | 3.6E−05 | 3.1E−11 |
| hHA-002 | 1.0E+06 | 5.5E−05 | 5.3E−11 |
| hHA-003 | 8.7E+05 | 3.1E−05 | 3.6E−11 |
| hHA-004 | 1.1E+06 | <1E−6 | <9.4E−13 |
| hHA-005 | 1.4E+06 | 2.1E−04 | 1.5E−10 |
| hHA-006 | 9.4E+05 | 3.0E−05 | 3.2E−11 |
| hHA-007 | 5.1E+05 | <1E−6 | <2.0E−12 |
| hHA-008 | 6.6E+05 | <1E−6 | <1.5E−12 |
| hHA-009 | 6.8E+05 | 2.1E−04 | 3.1E−10 |
| hHA-010 | 6.6E+05 | 2.6E−05 | 3.9E−11 |
| hHA-011 | 7.0E+05 | 5.2E−05 | 7.4E−11 |
| hHA | 8.3E+05 | 1.7E−03 | 2.1E−09 |
| HA | 8.3E+05 | 2.5E−03 | 3.0E−09 |

TABLE 16
Binding Affinity (by BIAcore) of
anti-HJV antibodies to Human RGMc

| Ab Name | Kₐ (1/MS) | K_{d} (1/s) | KD (M) |
|---|---|---|---|
| hHA-001 | 1.08E+07 | 1.65E−03 | 1.6E−10 |
| hHA-002 | 7.65E+06 | 1.70E−03 | 2.25E−10 |
| hHA-003 | 8.10E+06 | 2.40E−03 | 3E−10 |
| hHA-004 | 7.85E+06 | 1.04E−03 | 1.3E−10 |
| hHA-005 | 4.15E+07 | 1.36E−02 | 3.2E−10 |
| hHA-006 | 6.65E+06 | 2.55E−03 | 3.85E−10 |
| hHA-007 | 5.05E+06 | 2.15E−03 | 4.25E−10 |
| hHA-008 | 3.95E+06 | 3.90E−04 | 1.05E−10 |
| hHA-009 | 1.55E+07 | 1.07E−02 | 7.1E−10 |
| hHA-010 | 9.90E+06 | 4.40E−03 | 4.45E−10 |
| hHA-011 | 5.05E+06 | 1.40E−03 | 2.85E−10 |
| hHA | 8.3E+05 | | 2.1E−08 |

Within these antibodies, at lease hHA-004, hHA-008, hHA-009 and hHA-011 showed strong binding to human RGMc, and were selected for further testing. Sensorgrams by BIAcore analysis of antibodies HA, hHA-004, hHA-008, hHA-009 and hHA-011 are shown in FIGS. 1B-1G. hHA-008 was further tested for its binding affinity to human RGMa, Cyno RGMa, Rat RGMa, human RGMc, Cyno RGMc, Rat RGMc, and human RGMb, and the respective binding affinity is shown in Table 7. hHA-008 showed high affinity binding to human RGMa and RGMc with strong cross-reactivity to cyno and rodent species.

TABLE 17
Binding affinities of hHA-008 to human RGMa, Cyno RGMa, Rat
RGMa, human RGMc, Cyno RGMc, Rat RGMc, and Human RGMb.

| RGM antigen in | hHA-008 | | | hHA | | |
|---|---|---|---|---|---|---|
| solution | ka (1/Ms) | kd (1/s) | KD (M) | ka (1/Ms) | kd (1/s) | KD (M) |
| Human RGMa | 4.9E+05 | 3.6E−05 | 7.4E−11 | 2.7E+05 | 2.3E−03 | 8.3E−09 |
| Cyno RGMa | 5.5E+05 | 3.5E−04 | 6.4E−10 | 2.1E+05 | 6.6E−04 | 3.1E−09 |
| Rat RGMa | 3.1E+05 | 3.6E−05 | 1.2E−10 | 2.3E+05 | 2.1E−03 | 9.1E−09 |
| Human RGMc | 2.5E+06 | 2.9E−04 | 1.2E−10 | 1.4E+06 | n/a | 4.0E−08* |
| cyno RGMc | 2.6E+06 | 3.1E−04 | 1.2E−10 | 1.8E+06 | n/a | 3.2E−08* |
| Rat RGMc | 1.4E+06 | 3.2E−04 | 2.3E−10 | | | 6.6E−08** |

| | | |
|---|---|---|
| Human RGMb | no significant binding | no significant binding |

*Data for the hHA with human and cyno RGMc was fitted to a two-state model. kₐ₁ represents the Ab Ag association in a two-state binding model
**Data for the hHA with Rat RGMc was fitted to a steady state affinity model

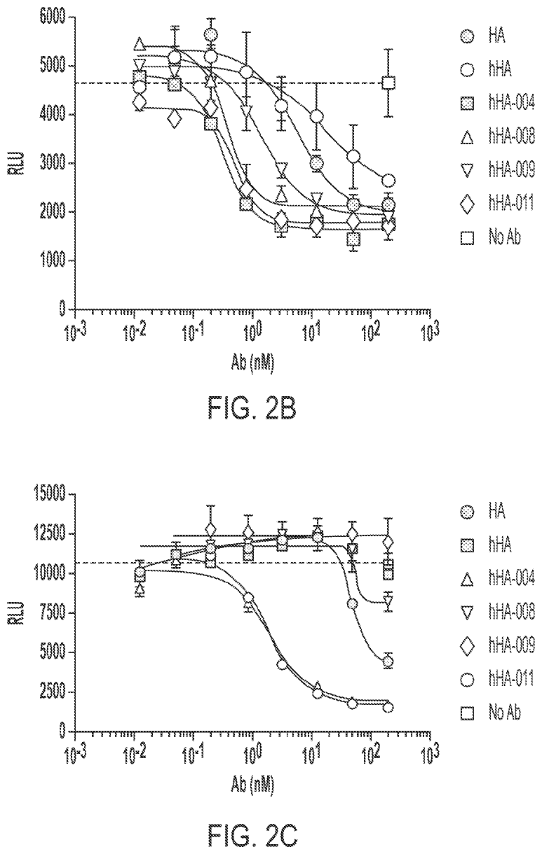

The RGMc (HJV) BMP reporter gene assay was used for screening and characterization potency of anti-HIV antibodies in blocking membrane-bound RGMc induced BMP/Smad1/5/8 signaling. The assay is directly related to the mechanism of action of the anti-HJV mAbs in inhibiting RGMc/BMP/Smad1/5/8 signaling pathway that is responsible for induction of iron hormone hepcidin gene expression. The principle of HJV BMP reporter assay is illustrated in FIG. 2A.

The BRE-Luc reporter gene vector initially described by Korchynskyi and Dijke (J. Biol. Chem. 2002; 277: 4883c) was used to transiently transfect porcine kidney epithelial cell line LLC-PK1 with or without co-transfection of an RGMa expression vector by Babbit et al (J. Biol. Chem. 2005; 280:29820) to determine if RGMa expression modulates BMP signaling. In BRE-Luc transiently transfected cells, RGMa was demonstrated to enhance BMP signaling through the Smad1/5/8 signaling pathway, consistent with a role for RGMa as a BMP co-receptor. All RGM members (RGMa, RGMb, and RGMc) act as BMP co-receptor and enhance BMP signaling. The BRE-Luc reporter vector was constructed and established the RGM BMP reporter gene assay in HEK293 cells. Human RGMc expression vector used in the assay was pcDNA-hRGMc.

HEK293 cells were cultured in growth media (base media DMEM (Invitrogen catalog #11965-092) containing 10% fetal bovine serum (Gibco #10438-026) and 1% sodium pyruvate (Invitrogen, catalog #11360-070)). To prepare cells for transfection, 9×10⁶ cells HEK293 cells were plated in 10 cm dishes and incubated at 37° C., 5% CO₂ for 6 hours. The cells were then transfected using PolyJet (μL):DNA (μg) ratio of 2:1, specifically 20 μl PolyJet (SignaGen, cat #SL100688) with 5 μg pGL[luc2P/BRE/Hygro] DNA (Abbvie) and 5 μg pcDNA-hRGMc (Abbvie) for 16 hours at 37° C., 5% CO₂. The media was replaced with fresh HEK293 growth media for 6 hours. The cells were then trypsinized with TrpLE (ThermoFisher, cat #12605010), counted, and plated in 96-well white assay plates (Thermo Scientific Nunclon delta F96, cat #136102) at 1×10⁵ cells per well. The cells were treated with anti-RGMc mAb, anti-BMP2/4 mAb (R&D Systems, cat #MAB3552) (as a positive control), or an isotype control mAb for 16 hours at 37° C., 5% CO₂. Luciferase activity was detected using the One-Glo Luciferase kit (Promega, cat #E6120) and measurement on a TopCount luminescence counter. The results showed that anti-HJV antibodies dose-dependently inhibit RGMc potentiated BMP signaling (FIG. 2B). The IC50 (nM) of each of the antibodies tested in inhibiting RGMc signaling in BMP reporter assay are shown in Table 18.

TABLE 18
IC50 of the anti-HJV antibodies in BMP reporter assay for RGMc.

| | HA | hHA | hHA-004 | hHA-008 | hHA-009 | hHA-011 |
|---|---|---|---|---|---|---|
| IC50 (nM) | 5.713 | 17.08 | 0.3124 | 0.3772 | 1.362 | 0.0554 |

Further, the anti-HJV antibodies were tested for their ability to inhibit RGMa signaling in BMP reporter assays. The hHA-004 and hHA-011 showed potent inhibition on membrane-bound human RGMa in RGMa BMP reporter gene assay, whereas hHA-008 and hHA-009 showed no to minimal inhibition on RGMa activity. The hHA antibody showed no inhibition to membrane bound RGMa. The rat mAb HA showed inhibition on RGMa but to much less extent as compared to hHA-004 and hHA-011. FIG. 2C. The IC50 (nM) of each of the antibodies tested in inhibiting RGMa signaling in BMP reporter assay are shown in Table 9.

TABLE 19
IC50 of the anti-HJV antibodies in BMP reporter assay for RGMa

| | HA | hHA-004 | hHA-008 | hHA-009 | hHA-011 |
|---|---|---|---|---|---|
| IC50 (nM) | 47.63 | 2.014 | ~56.54 | N/A | 1.657 |

The above data showed that the potency of the anti-HIV antibodies in neutralizing membrane RGMc in BMP reporter assay correlates with binding affinity on soluble RGMc. A summary of the anti-HJV antibody binding affinity to soluble human RGMc, and their capability to inhibit membrane bound RGMc and RGMa signaling are shown in Table 20.

TABLE 20
Anti-HJV antibody binding affinity to soluble human RGMc, and their
capability to inhibit membrane bound RGMc and RGMa signaling.

| | | | | Potency on membrane | Potency on membrane |
|---|---|---|---|---|---|
| | Binding Affinity (BIAcore) on soluble | | | hRGMc (RGMc/BMP | hRGMa (RGMa/BMP |
| | hRGMc | | | reporter assay) | reporter assay) |
| IgG | Ka (1/MS) | Kd (1/s) | K_{D} (M) | IC₅₀ (nM) | IC₅₀ (nM) |
| hHA-004 | 7.85E+06 | 1.04E−03 | 1.30E−10 | 0.31 | 2 |
| hHA-008 | 3.95E+06 | 3.90E−04 | 1.05E−10 | 0.37 | Minimal inhibition |
| hHA-009 | 1.55E+07 | 1.07E−02 | 7.10E−10 | 1.36 | No inhibition |
| hHA-011 | 5.05E+06 | 1.40E−03 | 2.85E−10 | 0.52 | 1.7 |
| hHA | 8.3E+05 | n/a | 2.9E−08 | 17 | No inhibition |
| | (kₐₗ)* | | | | |
| HA | 3.40E+06 | 5.00E−03 | 1.60E−10 | 5.7 | 48 |

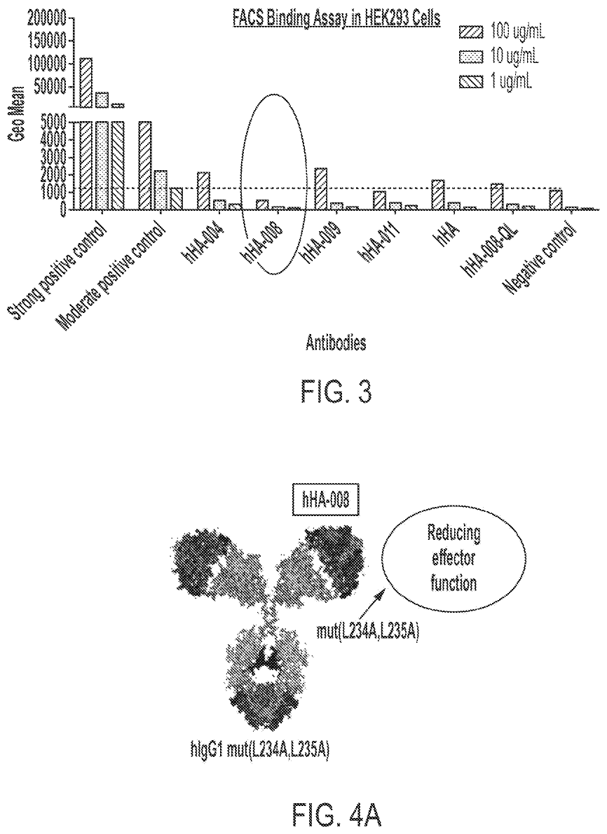

The antibodies, including hHA, hHA-004, hHA-008, hHA-008-QL, hHA-009, and hHA-011 were tested for non-specific cell binding to HEK293 cells by FACS analysis. The data showed hHA and its hHA-008, hHA-008-QL and hHA-011 showed no to minimal non-specific cell binding on HEK293 cells at conc. up to 100 μg/ml. hHA-004 and hHA-009 showed some non-specific cell binding at higher concentrations, but to a much less extent compared to positive control IgGs (FIG. 3).

### Example 2: Generation of hHA-008-QL

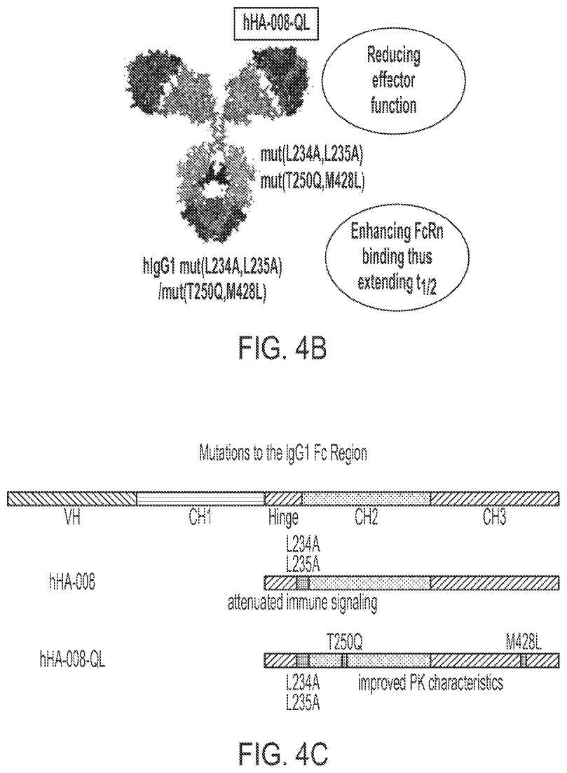

hHA-008-QL was developed to prolong IgG serum half-line (t_{1/2}). Previous studies have shown that neonatal Fc receptor (FcRn) protects IgG from catabolism thereby increasing IgG serum half-life. Accordingly, the Fc portion of hHA-008-QL was engineered to have T250Q and M428L mutations (QL mutations) such that it has enhanced binding to FcRn. FIGS. 4A and 4B illustrates the structure of hHA-008 and hHA-008-QL respectively. A further illustration of hHA-008 and hHA-008-QL is shown in FIG. 4C.

First, hHA-008-QL was tested for its RGMa and RGMc binding capabilities, and the data showed that hHA-008-QL had binding affinities to RGMa and RGMc comparable to that of hHA-008 (Table 21).

TABLE 21
hHA-008-QL and hHA-008 binding affinities to RGMa and RGMc

| | huRGMa | | | huRGMc | | |
|---|---|---|---|---|---|---|
| IgG | ka (1/Ms) | kd (1/s) | KD (M) | ka (1/Ms) | kd (1/s) | KD (M) |
| hHA-008-QL | 4.3E+05 | 3.3E−05 | 7.7E−11 | 2.7E+06 | 3.3E−04 | 1.2E−10 |
| (hIgG1mut(LALA)/QL/k) | | | | | | |
| hHA-008 | 5.4E+05 | 4.2E−05 | 7.8E−11 | 3.0E+06 | 3.2E−04 | 1.1E−10 |
| (hIgG1mut(LALA)/k | | | | | | |

### Example 3: Immunogenicity Studies of hHA-008 and hHA-008-QL

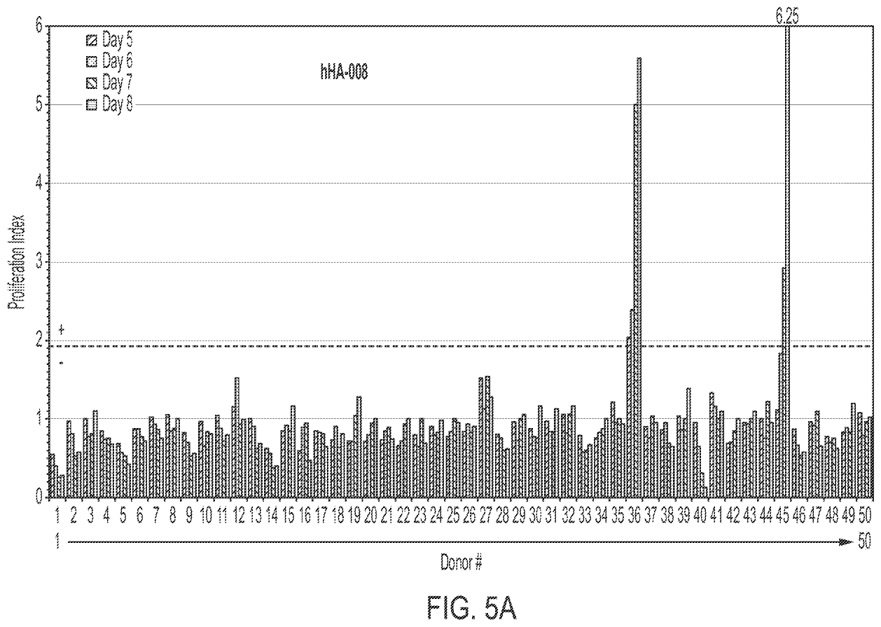

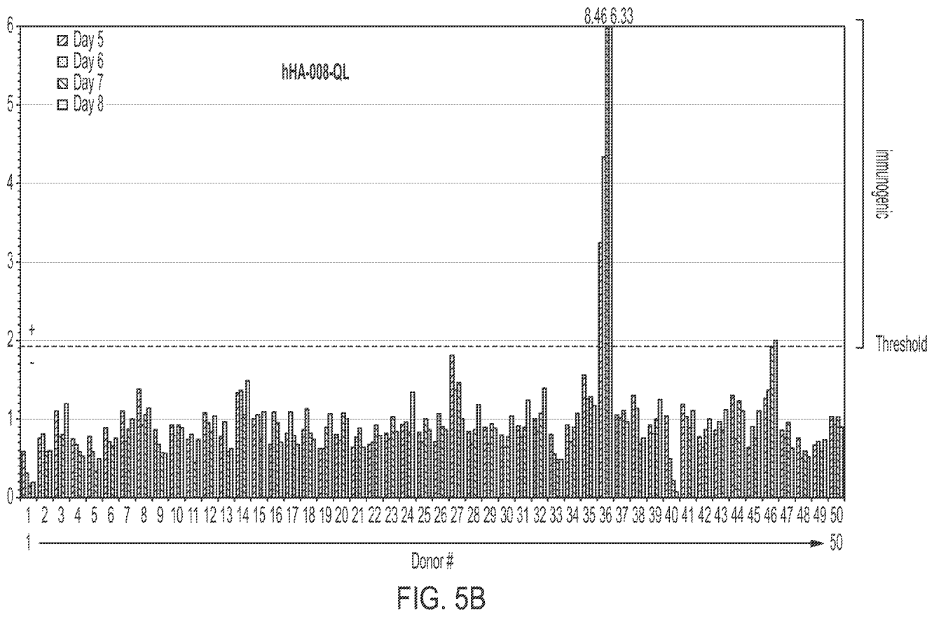

hHA-008 and hHA-008-QL were tested for their immunogenicity using peripheral blood mononuclear cells (PBMCs) from 50 donors (representing all major HLA-DR and HLA-DQ haplotypes) challenged with hHA-008 or hHA-008-QL in a CD4+ T cell response assay. The results showed only 4% of the 50 donors (2/50) showed a T cell response suggesting that both antibodies have low risk of immunogenicity. In this assay, Herceptin were used as negative control, to which 8% of the donors had a T cell response. Bydureon and keyhole limpet haemocyanin (KLH) were used as positive control. 32% of the donors showed T cell response to Bydureon and 100% donor had a T cell response to KLH. FIG. 5 showed T cell response in 50 donors for hHA-008 and hHA-008-QL.

Further, FcγR binding was tested for hHA-008 and hHA-008-QL as another parameter for immunogenicity. The control group, wild-type irrelevant IgG1 had significant binding to both the high and low affinity Fc gamma receptors. As expected, the binding of the wild-type irrelevant IgG4 is significantly lower than that of wild-type irrelevant IgG1. Binding to the high affinity and low affinity receptors is significantly reduced for both hHA-008 and hHA-008-QL compared to wild-type IgG1, suggesting low immunogenicity for both antibodies.

### Example 4: Pharmacokinetics/Pharmacodynamics (PK/PD) Modeling of hHA-008 and hHA-008-QL

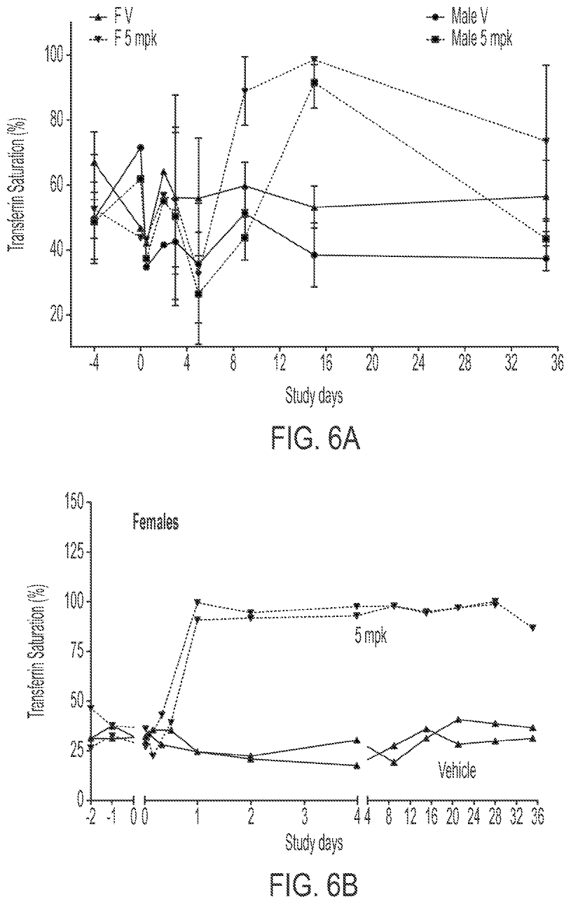

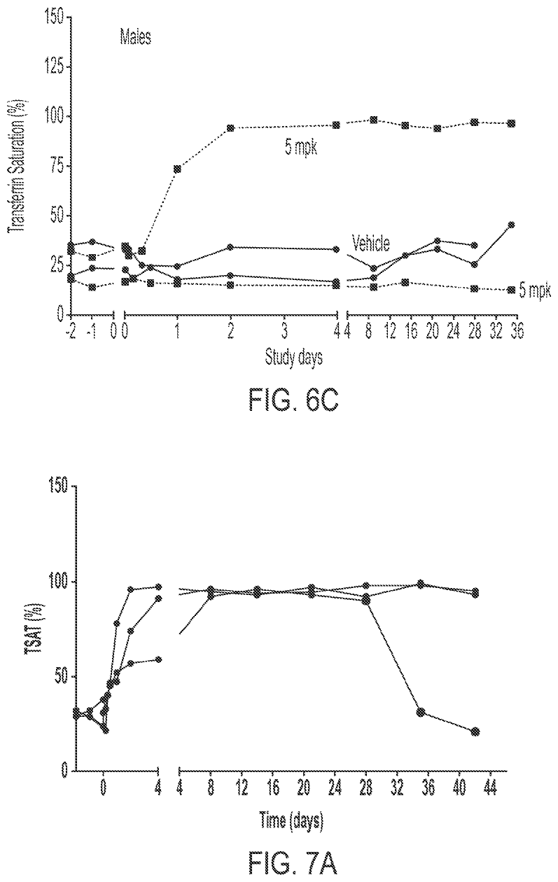

In an initial study for PK/PD of hHA-008, male rats were treated with a single dose of hHA-008 at 5 mg per kilogram of body weight (mpk) by intravenous injection. Transferrin saturation (TSAT %; determined as the percentage of serum iron over serum total iron binding capacity) were tested over time. The data showed that maximal effect of increased TSAT occurred between 4-8 days post treatment (FIG. 6A).

Further, a similar study was conducted in non-human primate Cynomolgus macaque (cyno). Both female (n=2) and male (n=2) cynos received a single dose of hHA-008 at 5 mg per kilogram of body weight (mpk) by intravenous injection. Transferrin saturation (TSAT %) were tested over time. Both female cynos showed maximum effect of increased TSAT % about 1-4 days after injection (FIG. 6B). One of the males showed the same effect, while the other one didn't respond to hHA-008 treatment (FIG. 6C). It was subsequently determined by examining the hematological profile of this male, that the lack of response was the result of this cyno having plasma Hepcidin-25 level that was below the limit of detection (below 2 ng/mL). Further, before the injection, this male cyno had a lower baseline TSAT % and serum iron level than other cynos in the group: two days before the injection, it had a baseline TSAT % level at 18% and a serum iron level at 77 μg/dL; one day before the injection, it had a baseline TSAT % level at 14% and a serum iron level at 61 μg/dL. The low baseline TSAT % and serum iron levels, in combination with the low Hepcidin-25 levels, was indicative that this male cyno had absolute iron deficiency instead of functional iron deficiency.

Further studies examining the effects of hHA-008 administration in more than 70 cynos revealed that animals having normal baseline serum iron levels (e.g., in the range of 80 μg/dL to 180 μg/dL) and hepcidin-25 levels greater than 2 ng/mL were responsive to hHA-008 treatment in that they exhibited pronounced increase in TSAT % levels shortly after injection (e.g., within 1-2 days post treatment) in response to reduced hepcidin-25 expression.

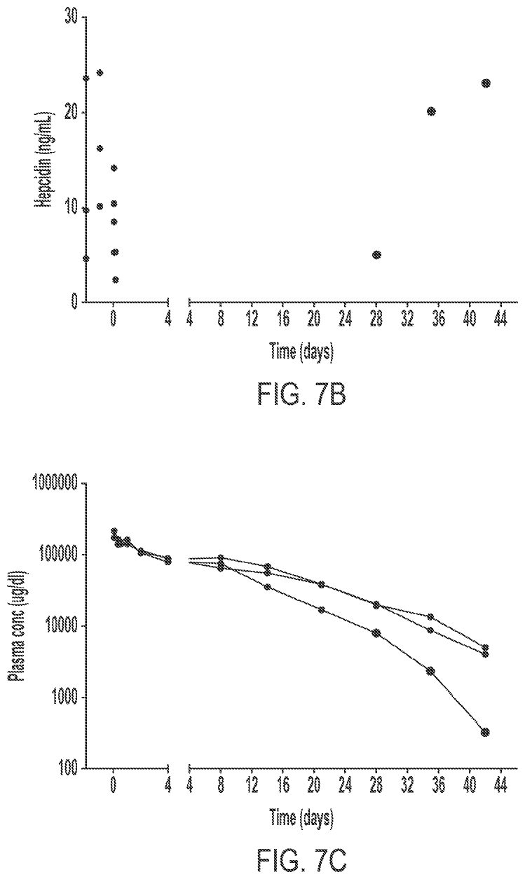

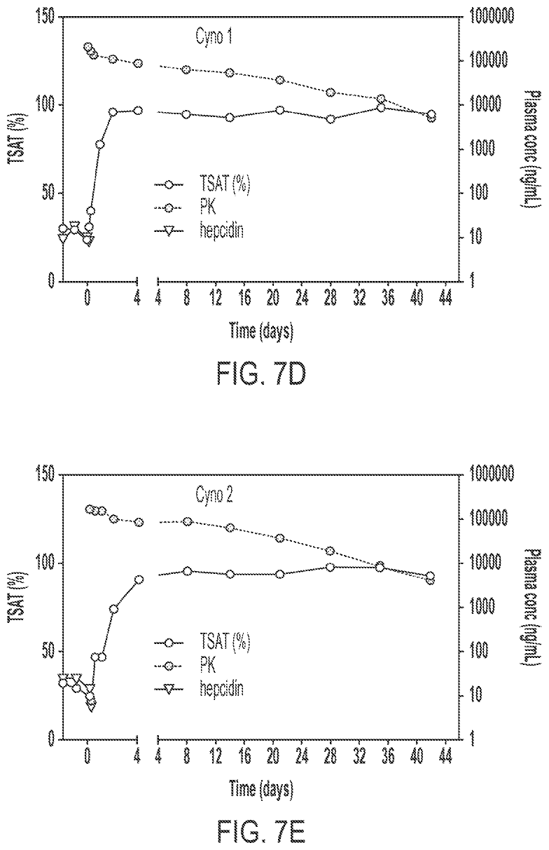

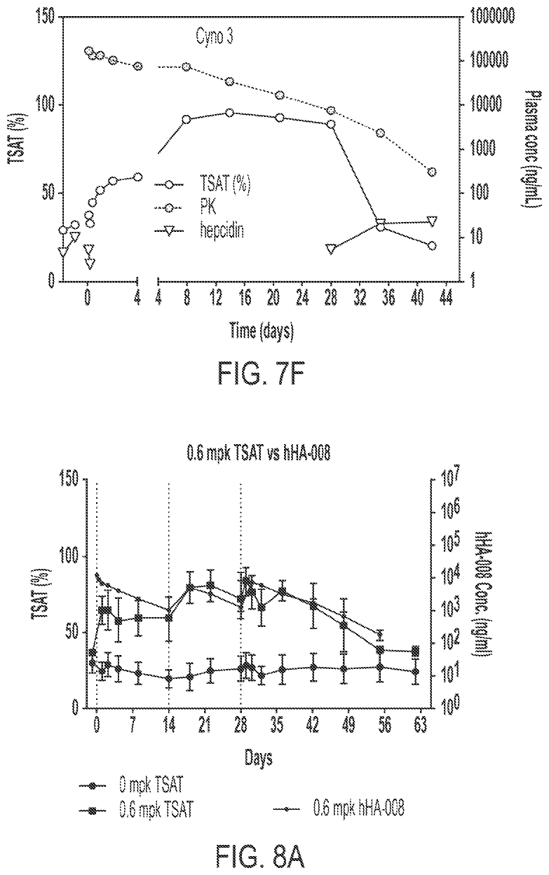

Additional experiments were conducted in cynos. Single dose of 6 mpk of hHA-008 were administered to three cynos via intravenous injection, and TSAT % (determined as serum iron over total iron binding capacity), plasma hepcidin-25 concentration, and plasma hHA-008 concentrations were tested for each animal and time point. Similarly, the data showed that the maximum effect of increased TSAT % occurred 1-4 days after injection (Tₘₐₓ=1-4 days), which was consistent with the increase of hHA-008 concentrations. One of the animals had a drastic decline of TSAT % around day 34 (FIG. 7A), which was consistent with the declining of plasma hHA-008 concentration around that time. Plasma hepcidin-25 concentration correlated inversely with the concentrations of hHA-008, in that hepcidin-25 was undetectable after antibody injection, and for the animal which had the drastic decline of TSAT %, hepcidin-25 level increased around the same time (FIG. 7B). In this cyno, hHA-008 had a plasma tin of about 5 days. The animal showed decline of TSAT % and increased hepcidin-25 levels at approximately day 34, which correlated well with the decrease of hHA-008 from plasma (FIG. 7C). Tin of ˜7 days in Cyno supports at least one/month dosing frequency in human.

Moreover, hHA-008 had showed robust PK/PD correlation of PK (plasma antibody concentration) to TSAT % and plasma hepcidin-25 concentrations. The results of each tested Cyno are shown in FIG. 7D (Cyno 1), FIG. 7E (Cyno 2), and FIG. 7F (Cyno 3). hHA-008 showed a t_{1/2} of 10.3 days in Cyno 1, t_{1/2} of 8.8 days in Cyno 2, and tin of 5.1 days in Cyno 3. Hepcidin-25 level drops to undetectable (<2 ng/ml) after hHA-008 treatment. Interestingly, return of hepcidin-25 level to circulation was well correlated with the tin of hHA-008 in Cyno 3 (FIG. 7F).

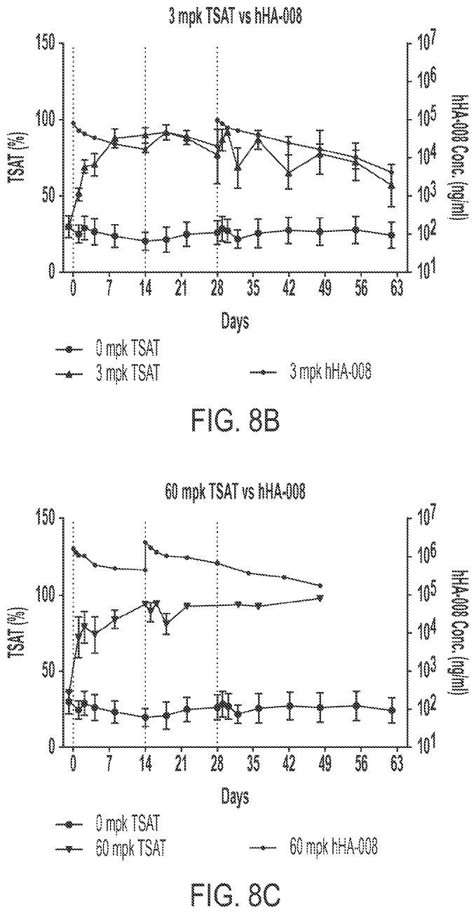

hHA-008 antibody modulates TSAT % in a dose-dependent manner. Multiple dose studies were conducted in Cynos. The cynos (n=4 per dose level with 2 males and 2 females) were treated with either 0 (vehicle control), 0.6 mpk hHA-008, 3 mpk hHA-008, or 60 mpk hHA-008. The resulting concentrations of hHA-008 and the corresponding TSAT % response are presented in FIG. 8A, FIG. 8B and FIG. 8C for the 0.6, 3 and 60 mpk treatments respectively, all plotted vs the vehicle control. Cynos were dosed every 14 days. Dotted lines represent dose day. TSAT % increased after dosing, and the percent modulation was consistent with the dose levels: at 0.6 mpk. TSAT reached ˜60%, and at 3 mpk and 60 mpk. TSAT % was saturated, indicating that hHA-008 modulates TSAT % in a dose dependent manner. Further, after the first dose at 0.6 mpk, TSAT % levels were maintained at ˜60%, while at higher dose levels, TSAT % reached 100%, suggesting that TSAT can be modulated by selection of appropriate dose-level/regimen (FIGS. 8A-8C).

### Example 5: hHA-008-QL Confers Longer Serum Half-Life and Takes Longer Time to Reach Maximal Effect Compared to hHA-008

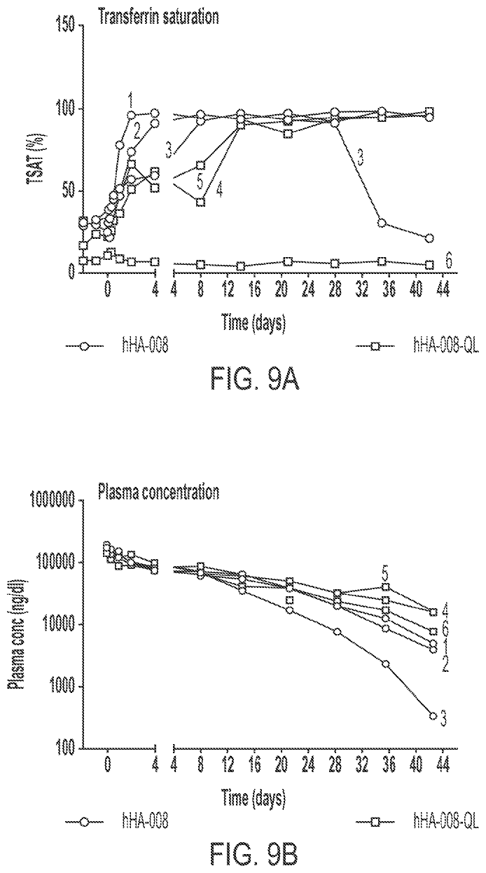

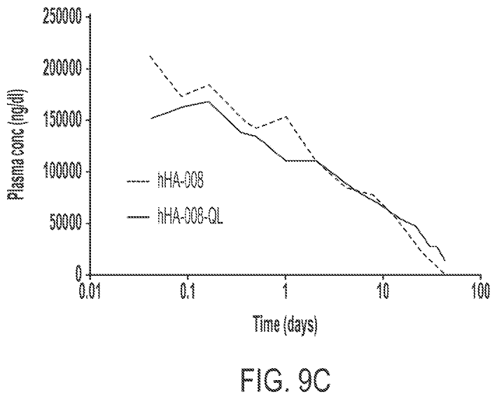

This study was designed to administer hHA-008 or hHA-008-QL at 6 mpk intravenously to Cynos (n=3). Samples were collected from each animal at 48 and 24 hr prior to treatment, and at 0, 0.04, 0.08, 0.167, 0.333, 0.5, 1, 2, 4, 8, 14, 21, 28, 35 and 42 days after treatment. TSAT % and plasma hepcidin-25 level were measured in each sample. The data showed that hHA-008 took shorter time to reach maximal effect (Tₘₐₓ) of increased TSAT % as compared to hHA-008-QL (FIG. 9A). Note that there was a non-responder in animals treated with hHA-008-QL. As discussed above, the non-responder had low hepcidin-25 levels and low serum iron at base line prior to antibody treatment. On the other hand, hHA-008-QL had longer serum half-life (T1/2=12.1 days) compared to hHA-008 (T1/2=6.76 days) (FIG. 9B). Plasma concentrations of both antibodies were measured over time, as shown in FIG. 9C. Peak plasma concentrations were determined. A summary of the data between hHA-008 and hHA-008-QL are shown in Table 22.

TABLE 22
hHA-008 v. hHA-008-QL

| Parameter | Units | hHA-008 | hHA-008-QL |
|---|---|---|---|
| Cₘₐₓ | μg/mL | 211.15 | 168.4 |
| Tₘₐₓ | days | 0.083 | 2 |
| T½ | days | 6.76 | 12.1 |

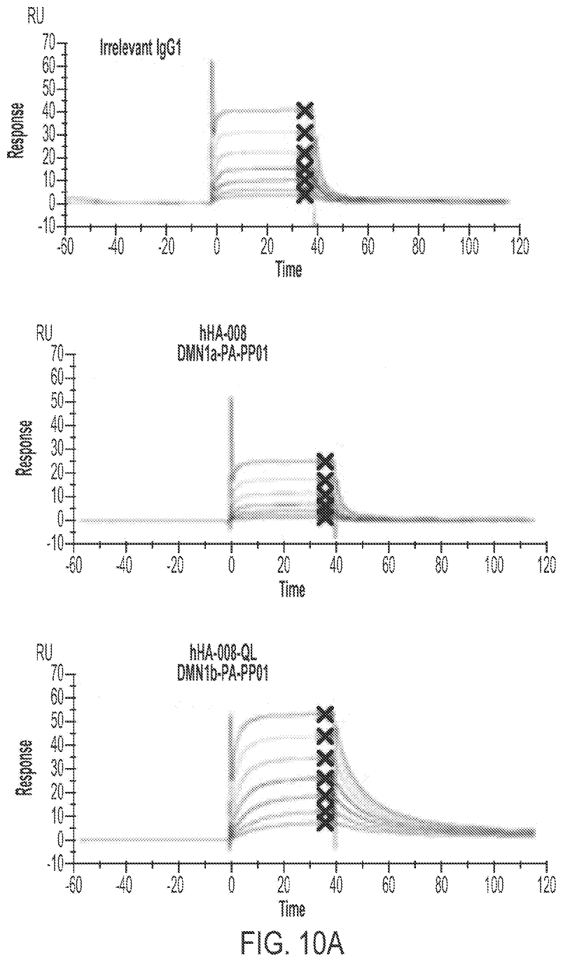

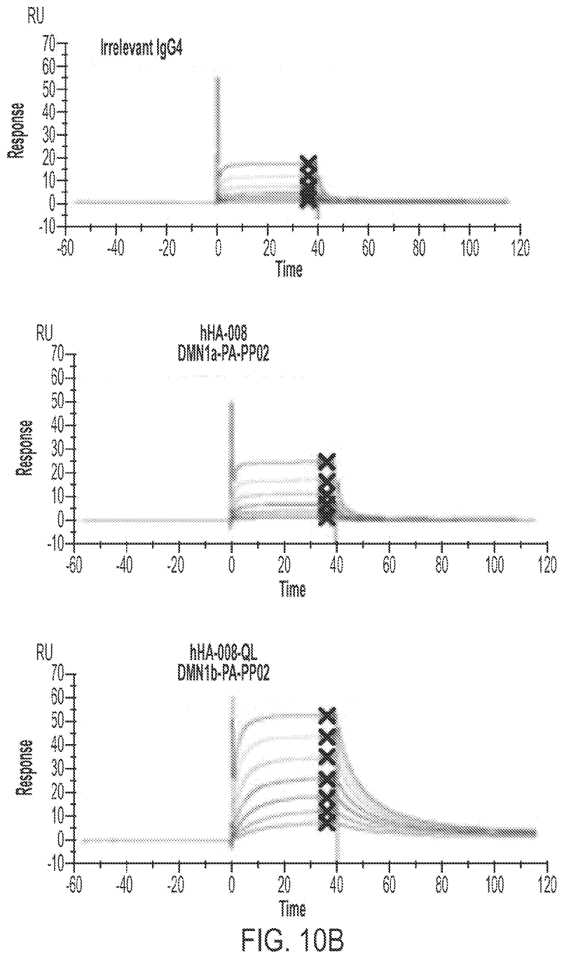

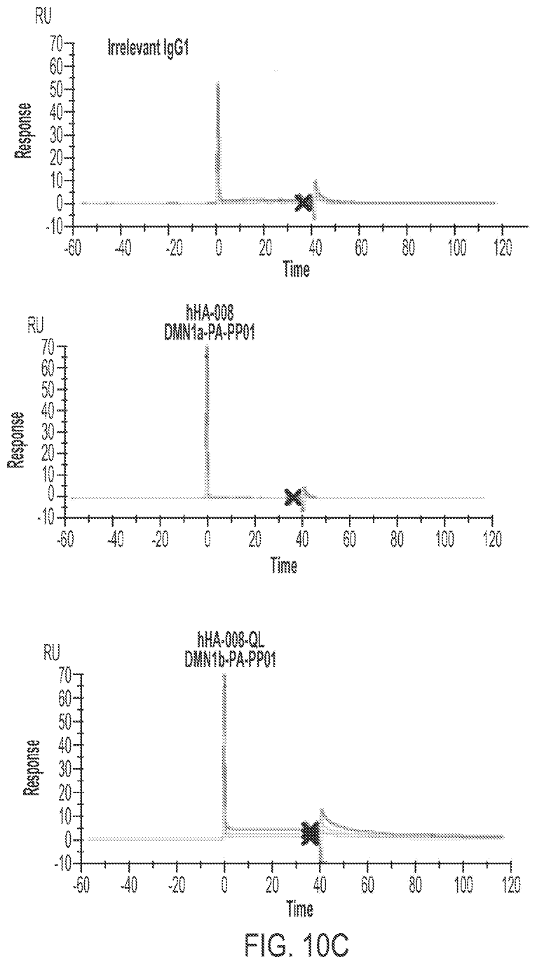

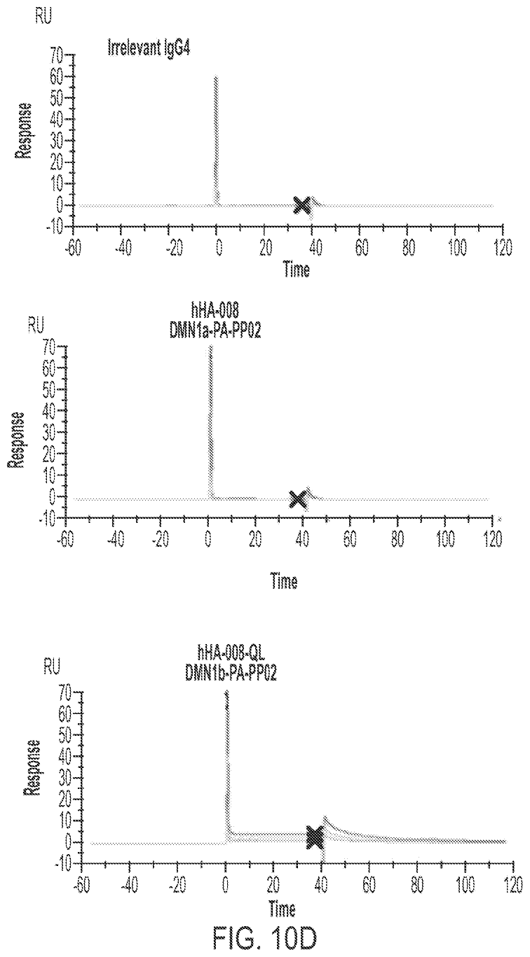

To investigate whether the longer serum life for hHA-008-QL was attributed to higher affinity to FcRn, both antibodies were analyzed for binding to FcRn at pH 6.0 and pH 7.4 using BIAcore. Non-specific IgG1 and IgG4 were used as controls. The dissociation constant (KD) of each of the antibody and the controls to FcRn at pH 6.0 and 7.4 were shown in Table 23, and the response curve were shown in FIGS. 10A-10B. No binding was observed of neither antibodies at pH 7.4 (FIG. 10B).

TABLE 23
KD for FcRn at pH 6.0 and pH 7.4

| | KD (M) at pH 6.0 | KD (M) at pH 7.4 |
|---|---|---|
| IgG1 | 1.64E−06 | no binding |
| IgG4 | 3.64E−06 | no binding |
| hHA-008 | 2.95E−06 | no binding |
| hHA-008 | 2.96E−06 | no binding |
| hHA-008-QL | 9.00E−07 | no binding |
| hHA-008-QL | 8.82E−07 | no binding |

The mutations L247A and L248A (Kabat) (L234A and L235A, EU Numbering) do not appear to significantly affect FcRn binding. The overall affinity and response (RMax) for binding of FcRn at pH 6.0 is increased for hHA-008-QL compared to hHA-008, suggesting that the QL mutation confers the longer t1/2 via the binding to the receptor.

### Example 6: hHA-008 Decreases IL-6 Induced Hepcidin-25 Expression in Non-Human Primates

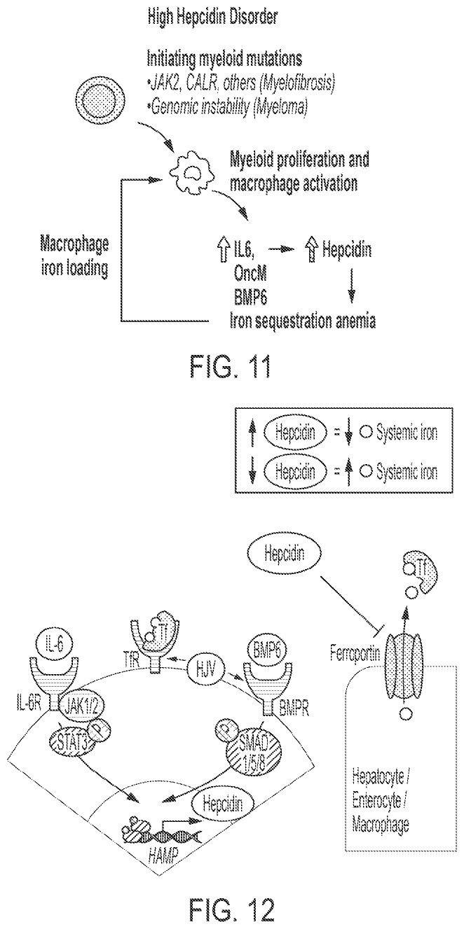

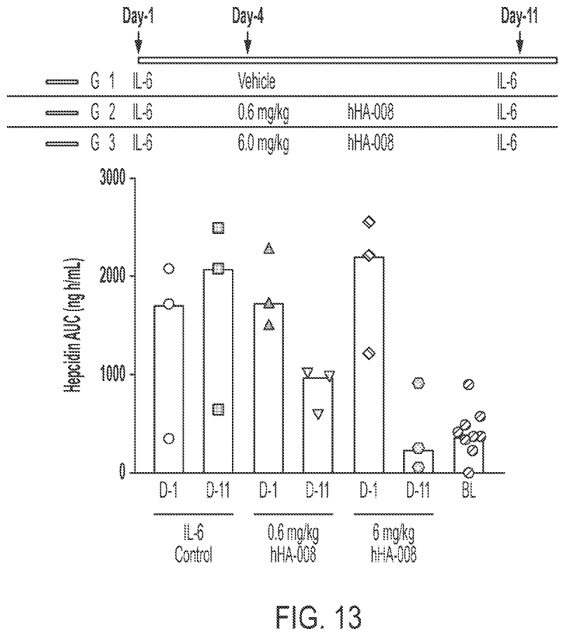

As depict in FIG. 11, in iron sequestration anemia, pro-inflammatory cytokines that induce hepcidin synthesis, such as IL-6 and oncostatin-M, are typically increased and associated with iron sequestration, macrophage iron loading, as well as myeloid proliferation and macrophage activation. To test whether IL-6 indeed increases hepcidin expression and whether anti-HJV antibody is capable of inhibiting hepcidin expression induced by IL-6 in non-human primates, cynos were challenged with IL-6 on day 1, and divided into three groups. On day 4, cynos in Group 1 received vehicle control, cynos in Group 2 received hHA-008 antibody at 0.6 mg/kg, and cynos in Group 3 received hHA-008 antibody at 6.0 mg/kg. On day 11, cynos in all three groups were challenged with IL-6 again, and plasma hepcidin-25 in all cynos was measured. As shown in FIG. 13, IL-6 challenge increased plasma hepcidin-25 concentrations on Day 1, compared to pre-challenge baseline (BL) in all three groups of cynos. After the second IL-6 challenge on Day 11, cynos in group 1 showed an increase in plasma hepcidin-25 similar to that observed on Day 1. However, for cynos in groups 2 (0.6 mg/kg hHA-008) and 3 (6 mg/kg hHA-008), the presence hHA-008 prevented the IL-6 induced increase in plasma hepcidin-25 on Day 11 in a dose-dependent manner. That is, hHA-008 was effective in preventing inflammation-induced (IL 6) hepcidin increase in a dose-dependent manner in cynos. These results suggest that anti-HJV antibody are capable of inhibiting hepcidin expression induced by the IL-6 signaling pathway.

### Example 7: Epitope Mapping

Epitope mapping was performed on hHA-008 and hHA-008-QL, using 3720-RG-050 (Hemojuvelin (HJV) fragment, SEQ ID NO: 123).

Before epitope mapping, High-Mass MALDI mass spectrometry and chemical cross-linking was used to confirm no non-covalent aggregates of hHA-008 and hHA-008-QL and multimers of the 3720-RG-050 were detected in sample preparation.

In order to determine the epitopes of hHA-008 and hHA-008-QL on 3720-RG-050 with high resolution, 3720-RG-050/hHA-008 and 3720-RG-050/hHA-008-QL complexes were incubated with deuterated cross-linkers and subjected to multi-enzymatic cleavage. After enrichment of the cross-linked peptides, the samples were analyzed by high resolution mass spectrometry (nLC-LTQ-Orbitrap MS) and the data generated were analyzed using XQuest and Stavrox software.

### 3720-RG-050/hHA-008 (HJV Fragment/hHA-008)

After Trypsin, Chymotrypsin, ASP-N, Elastase and Thermolysin proteolysis of the protein complex 3720-RG-050/hHA-008 with deuterated d0d12, the nLC-orbitrap MS/MS analysis detected 8 cross-linked peptides between 3720-RG-050 and hHA008.

The sequences and positions of cross-links are presented in Table 8 below.

TABLE 8
Cross-linked peptides detected between 3720-RG-050 and hHA-008.
hHA-008 - Chymotrypsin and Thermolysin results in interlink between
hHA-008 complementarity determining regions and 3720-RG-050

| | | | Amino Acid | Amino acid | | | |
|---|---|---|---|---|---|---|---|
| | hHA-008 | 3720- | Residues of hHA- | Residues of | | | Identified |
| Enzyme | HC/hHA-008 LC | RG-050 | 008 HC/hHA-008 LC | 3720-RG-050 | nAA1 | nAA2 | on StavroX |
| Chymotrypsin | hHA-008_HC | SEQ ID | 48-53 | 166-189 | 52 | 170 | YES |
| | (SEQ ID NO: 38) | NO: 123 | | | | | |
| Chymotrypsin | hHA-008_LC | SEQ ID | 95-101 | 165-185 | 97 | 170 | YES |
| | (SEQ ID NO: 39) | NO: 123 | | | | | |
| Chymotrypsin | hHA-008_LC | SEQ ID | 95-101 | 165-185 | 98 | 171 | YES |
| | (SEQ ID NO: 39) | NO: 123 | | | | | |
| Chymotrypsin | hHA-008_LC | SEQ ID | 95-101 | 165-185 | 98 | 180 | YES |
| | (SEQ ID NO: 39) | NO: 123 | | | | | |
| Chymotrypsin | hHA-008_LC | SEQ ID | 95-101 | 165-185 | 98 | 182 | YES |
| | (SEQ ID NO: 39) | NO: 123 | | | | | |
| Thermolysin | hHA-008_HC | SEQ ID | 50-60 | 177-184 | 53 | 182 | YES |
| | (SEQ ID NO: 38) | NO: 123 | | | | | |
| Chymotrypsin | hHA-008_LC | SEQ ID | 95-101 | 165-185 | 98 | 183 | YES |
| | (SEQ ID NO: 39) | NO: 123 | | | | | |
| Thermolysin | hHA-008_HC | SEQ ID | 51-63 | 181-186 | 58 | 183 | YES |
| | (SEQ ID NO: 38) | NO: 123 | | | | | |

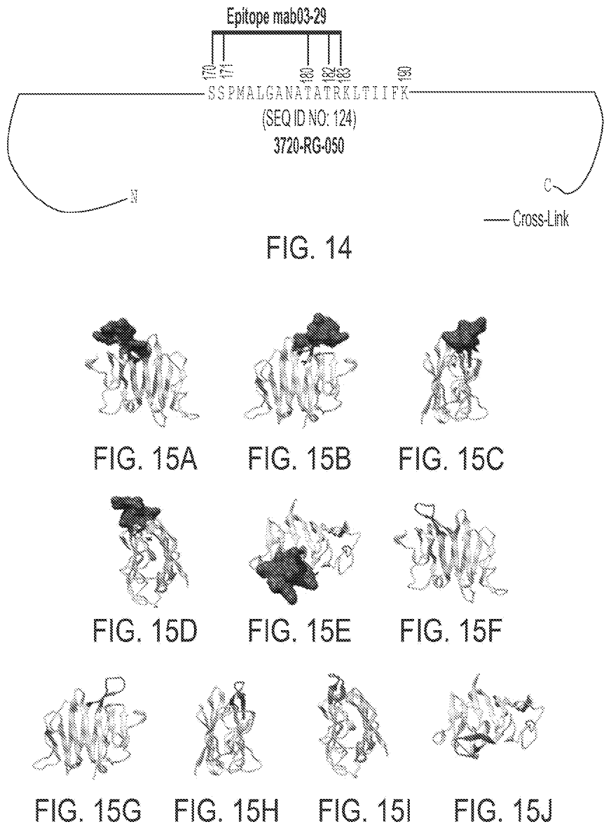

Using chemical cross-linking, High-Mass MALDI mass spectrometry and nLC-Orbitrap mass spectrometry, molecular interface between 3720-RG-050 and hHA-008 was characterized. FIG. 14 shows hHA-008 interacts with amino acids 170-183 (SSPMALGANATATR (SEQ ID NO: 121)) on 3720-RG-050. The interaction happens on amino acids 170, 171, 180, 182, 183 on 3720-RG-050. FIG. 15 shows the interaction of 3720-RG-050 and hHA-008. A 3720-RG-050 PDB structure was generated by homology using Swiss Model software. 3720-RG-050 amino acids 170-183 (SSPMALGANATATR (SEQ ID NO: 121)) of 3720-RG-050 sequence are shown in FIG. 15: ribbon/surface representation of front view (A); back view (B), side view 1 (C), side view 2 (D) and top view (E). F, G, H, I, J: ribbon representation of front view (F); back view (G), side view 1 (H), side view 2 (I) and top view (J).

### 3720-RG-050/hHA-008-QL (HJV Fragment/hHA-008-QL)

After Trypsin, Chymotrypsin, ASP-N, Elastase and Thermolysin proteolysis of the protein complex 3720-RG-050/hHA-008-QL with deuterated d0d12, the nLC-orbitrap MS/MS analysis detected 16 cross-linked peptides between 3720-RG-050 and hHA-008-QL.

The sequences and positions of cross-links are presented in Table 24 below.

TABLE 24
Cross-linked peptides detected between 3720-RG-050 and HA-008-QL.
hHA-008-QL- Trypsin, Chymotrypsin, AspN, Elastase and Thermolysin results
Interlink between hHA-008-QLcomplementarity determining regions and 3720-RG-050

| | | | Amino Acid | | | | |
|---|---|---|---|---|---|---|---|
| | | | Residues of | Amino acid | | | |
| | hHA-008-QL HC/ | | hHA-008-QL HC/ | Residues of | | | Identified |
| Enzyme | hHA-008-QL LC | 3720-RG-050 | hHA-008-QL LC | 3720-RG-050 | nAA1 | nAA2 | on StavroX |
| Chymotrypsin | hHA-008-QL_LC | SEQ ID NO: | 95-101 | 165-185 | 97 | 169 | YES |
| | (SEQ ID NO: 39) | 123 | | | | | |
| Chymotrypsin | hHA-008-QL_LC | SEQ ID NO: | 95-101 | 165-185 | 98 | 169 | YES |
| | (SEQ ID NO: 39) | 123 | | | | | |
| Chymotrypsin | hHA-008-QL_LC | SEQ ID NO: | 95-101 | 165-185 | 98 | 171 | YES |
| | (SEQ ID NO: 39) | 123 | | | | | |
| Chymotrypsin | hHA-008-QL_LC | SEQ ID NO: | 95-101 | 165-185 | 98 | 180 | YES |
| | (SEQ ID NO: 39) | 123 | | | | | |
| Chymotrypsin | hHA-008-QL_LC | SEQ ID NO: | 53-67 | 176-185 | 59 | 180 | YES |
| | (SEQ ID NO: 39) | 123 | | | | | |
| Chymotrypsin | hHA-008-QL_LC | SEQ ID NO: | 53-67 | 176-185 | 61 | 180 | YES |
| | (SEQ ID NO: 39) | 123 | | | | | |
| Chymotrypsin | hHA-008-QL_LC | SEQ ID NO: | 95-101 | 165-185 | 98 | 182 | YES |
| | (SEQ ID NO: 39) | 123 | | | | | |
| Chymotrypsin | hHA-008-QL_HC | SEQ ID NO: | 96-104 | 280-292 | 100 | 289 | YES |
| | (SEQ ID NO: 38) | 123 | | | | | |
| Thermolysin | hHA-008-QL_HC | SEQ ID NO: | 29-44 | 283-302 | 32 | 293 | YES |
| | (SEQ ID NO: 38) | 123 | | | | | |
| Elastase | hHA-008-QL_HC | SEQ ID NO: | 31-42 | 290-295 | 32 | 294 | YES |
| | (SEQ ID NO: 38) | 123 | | | | | |
| Thermolysin | hHA-008-QL_HC | SEQ ID NO: | 29-44 | 283-302 | 32 | 295 | YES |
| | (SEQ ID NO: 38) | 123 | | | | | |
| Trypsin | hHA-008-QL_HC | SEQ ID NO: | 39-58 | 292-299 | 52 | 295 | YES |
| | (SEQ ID NO: 38) | 123 | | | | | |
| Trypsin | hHA-008-QL HC | SEQ ID NO: | 20-43 | 295-300 | 32 | 297 | YES |
| | (SEQ ID NO: 38) | 123 | | | | | |
| Thermolysin | hHA-008-QL_HC | SEQ ID NO: | 29-44 | 283-302 | 30 | 300 | YES |
| | (SEQ ID NO: 38) | 123 | | | | | |
| Elastase | hHA-008-QL_HC | SEQ ID NO: | 31-42 | 294-302 | 32 | 300 | YES |
| | (SEQ ID NO: 38) | 123 | | | | | |
| Elastase | hHA-008-QL_HC | SEQ ID NO: | 31-42 | 294-302 | 38 | 300 | YES |
| | (SEQ ID NO: 38) | 123 | | | | | |

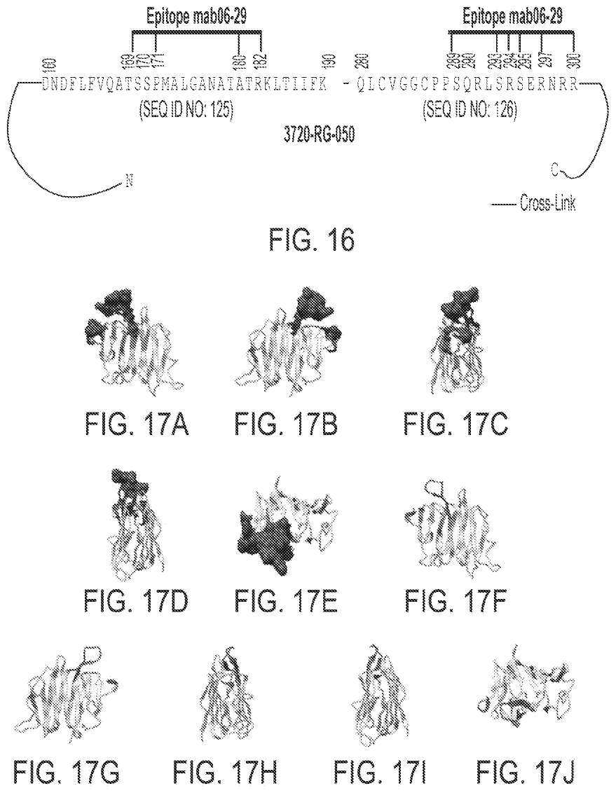

Using chemical cross-linking, High-Mass MALDI mass spectrometry and nLC-Orbitrap mass spectrometry, molecular interface between 3720-RG-050 and hHA-008-QL was characterized. FIG. 16 shows hHA-008-QL interacts with amino acids 169-182 (TSSPMALGANATAT (SEQ ID NO: 122)) and 289-300 (SQRLSRSERNRR (SEQ ID NO: 127)) of 3720-RG-050. The interaction happens on amino acids 169, 171, 180, 182; 289, 293, 294, 295, 297, 300 on 3720-RG-050. FIG. 17 shows the interaction 3720-RG-050/hHA-008-QL. A 3720-RG-050 PDB structure was generated by homology using Swiss Model software. 3720-RG-050 amino acids 169-182 (TSSPMALGANATAT (SEQ ID NO: 122) and 289-291 (SQR) are shown as FIG. 17: ribbon/surface representation of front view (A); back view (B), side view 1 (C), side view 2 (D) and top view (E). F, G, H, I, J: ribbon representation of front view (F); back view (G), side view 1 (H), side view 2 (I) and top view (J).

### Example 8: hHA-008-QL Decreases Circulating Transferrin Level

Circulating transferrin level can be used as an indicator of the iron level in the body along with other markers (e.g., total iron binding capacity, serum ferritin level, etc.). Circulating transferrin is an iron-transport protein that reflects both protein and iron status. Transferrin increases with iron deficiency and decreases when iron status improves (see, e.g., Litchford et al., NUTRITIONAL ISSUES IN THE PATIENT WITH DIABETES AND FOOT ULCERS, Levin and O'Neal's The Diabetic Foot (Seventh Edition), 2008; Ogun et al., Biochemistry, Transferrin, Treasure Island (FL): StatPearls Publishing; 2021 January).

To test whether circulating transferrin level decreases after hHA-008-QL treatment, baseline transferrin level was established for each health Cynomolgus macaque (cynos) in the experiment 11 days before hHA-008-QL dosing on Day 1. Cynos were dosed with 0 mg/kg, 3 mg/kg. 30 mg/kg or 160 mg/kg of hHA-008-QL on day 1, day 15 and day 29 (n=5 in each group). Blood from each cyno was collected 96 hours, 192 hours, and 336 hours after the dosing on day 1; 48 hours, 96 hours, 192 hours, and 336 hours after the dosing on day 15; and 48 hours after the dosing on day 29. Note that blood for the 336-hour timepoint after day 1 dosing was collected 2 hours before the day 15 dosing, and blood for the 336-hour time point after day 15 dosing was collect 2 hours before the day 29 dosing. Transferrin levels were measured in all samples for each cyno, and was compared to its own baseline to calculate the percent change at each time point. The data in Table 25 show that treatment of hHA-008-QL reduced circulating transferrin levels compared to the baseline level.

TABLE 25
Circulating transferrin levels and changes over baseline in cynos treated with hHA-008-QL.

| Day(s) | Males | | | | Females | | | |
|---|---|---|---|---|---|---|---|---|
| Relative to | 0 mg/ | 3 mg/ | 30 mg/ | 160 mg/ | 0 mg/ | 3 mg/ | 30 mg/ | 160 mg/ |
| Start Date | kg/dose | kg/dose | kg/dose | kg/dose | kg/dose | kg/dose | kg/dose | kg/dose |
| Transferrin | 241.96 ± | 227.82 ± | 239.14 ± | 227.58 ± | 241.96 ± | 227.82 ± | 239.14 ± | 227.58 ± |
| Day −11 | 14.74 | 22.69 | 18.75 | 10.21 | 14.74 | 22.69 | 18.75 | 10.21 |
| (mg/dL; | | | | | | | | |
| Baseline) | | | | | | | | |
| Transferrin | 259.28 ± | 223.74 ± | 226.74 ± | 217.74 ± | 259.28 ± | 223.74 ± | 226.74 ± | 217.74 ± |
| Day 1, 96 h | 10.46 | 20.49 ** | 13.64 ** | 9.88 ** | 10.46 | 20.49 ** | 13.64 ** | 9.88 ** |
| (mg/dL) | | | | | | | | |
| Day 1, 96 h | — | 0.86 | 0.87 | 0.84 | — | 0.86 | 0.87 | 0.84 |
| tCtrl | | | | | | | | |
| Transferrin | 263.06 ± | 224.92 ± | 226.62 ± | 221.64 ± | 263.06 ± | 224.92 ± | 226.62 ± | 221.64 ± |
| Day 1, 192 h | 19.77 | 21.07 ** | 11.34 ** | 10.96 ** | 19.77 | 21.70 ** | 11.34 ** | 10.96 ** |
| (mg/dL) | | | | | | | | |
| Day 1, 192 h | — | 0.86 | 0.86 | 0.84 | — | 0.86 | 0.86 | 0.84 |
| tCtrl | | | | | | | | |
| Transferrin | 260.68 ± | 223.36 ± | 228.82 ± | 225.72 ± | 260.68 ± | 223.36 ± | 228.82 ± | 225.72 ± |
| Day 1, 336 h | 20.70 | 24.75 * | 8.80 | 8.08 * | 20.70 | 24.75 * | 8.80 | 8.08 * |
| (mg/dL) | | | | | | | | |
| Day 1, 336 h | — | 0.86 | 0.88 | 0.87 | — | 0.86 | 0.88 | 0.87 |
| tCtrl | | | | | | | | |
| Transferrin | 259.76 ± | 218.06 ± | 225.86 ± | 221.78 ± | 259.76 ± | 218.06 ± | 225.86 ± | 221.78 ± |
| Day 15, 48 h | 18.52 | 28.28 * | 10.73 | 9.85 * | 18.52 | 28.28 * | 10.73 | 9.85 * |
| (mg/dL) | | | | | | | | |
| Day 15, 48 h | — | 0.84 | 0.87 | 0.85 | — | 0.84 | 0.87 | 0.85 |
| tCtrl | | | | | | | | |
| Transferrin | 253.88 ± | 211.48 ± | 219.52 ± | 209.26 ± | 253.88 ± | 211.48 ± | 219.52 ± | 209.26 ± |
| Day 15, 96 h | 19.14 | 22.48 ** | 11.17 * | 10.27 ** | 19.14 | 22.48 ** | 11.17 * | 10.27 ** |
| (mg/dL) | | | | | | | | |
| Day 15, 96 h | — | 0.83 | 0.86 | 0.82 | — | 0.83 | 0.86 | 0.82 |
| tCtrl | | | | | | | | |
| Transferrin | 254.78 ± | 215.88 ± | 219.92 ± | 211.30 ± | 254.78 ± | 215.88 ± | 219.92 ± | 211.30 ± |
| Day 15, 192 | 24.23 | 27.37 | 10.13 | 8.73 * | 24.23 | 27.37 | 10.13 | 8.73 * |
| h (mg/dL) | | | | | | | | |
| Day 15, 192 | — | 0.85 | 0.86 | 0.83 | — | 0.85 | 0.86 | 0.83 |
| h tCtrl | | | | | | | | |
| Transferrin | 268.04 ± | 222.58 ± | 235.52 ± | 231.42 ± | 268.04 ± | 222.58 ± | 235.52 ± | 231.42 ± |
| Day 15, 336 | 24.56 | 26.20 ** | 16.40 | 11.07 * | 24.56 | 26.20 ** | 16.40 | 11.07 * |
| h (mg/dL) | | | | | | | | |
| Day 15, 336 | — | 0.83 | 0.88 | 0.86 | — | 0.83 | 0.88 | 0.86 |
| h tCtrl | | | | | | | | |
| Transferrin | 236.04 ± | 198.48 ± | 209.30 ± | 198.12 ± | 236.04 ± | 198.48 ± | 209.30 ± | 198.12 ± |
| Day 29, 48 h | 14.11 | 19.87 ** | 14.41 * | 10.25 ** | 14.11 | 19.87 ** | 14.41 * | 10.25 ** |
| (mg/dL) | | | | | | | | |
| Day 29, 48 h | — | 0.84 | 0.89 | 0.84 | — | 0.84 | 0.89 | 0.84 |
| tCtrl | | | | | | | | |
| Transferrin | 252.24 ± | 209.24 ± | 225.80 ± | 209.90 ± | 252.24 ± | 209.24 ± | 225.80 ± | 209.90 ± |
| Day 29, 96 h | 12.39 | 24.21 ** | 225.80 | 8.04 ** | 12.39 | 24.21 ** | 15.43 | 8.04 |
| (mg/dL) | | | | | | | | |
| Day 29, 96 h | — | 0.83 | 0.90 | 0.83 | — | 0.83 | 0.90 | 0.83 |
| tCtrl | | | | | | | | |

Anova & Dunnett:
* = p ≤ 0.05;
** = p ≤ 0.01

### Example 9: hHA-008 Prevents Inflammation-Induced (IL-6) Iron Suppression in Cynomolgus Macaque

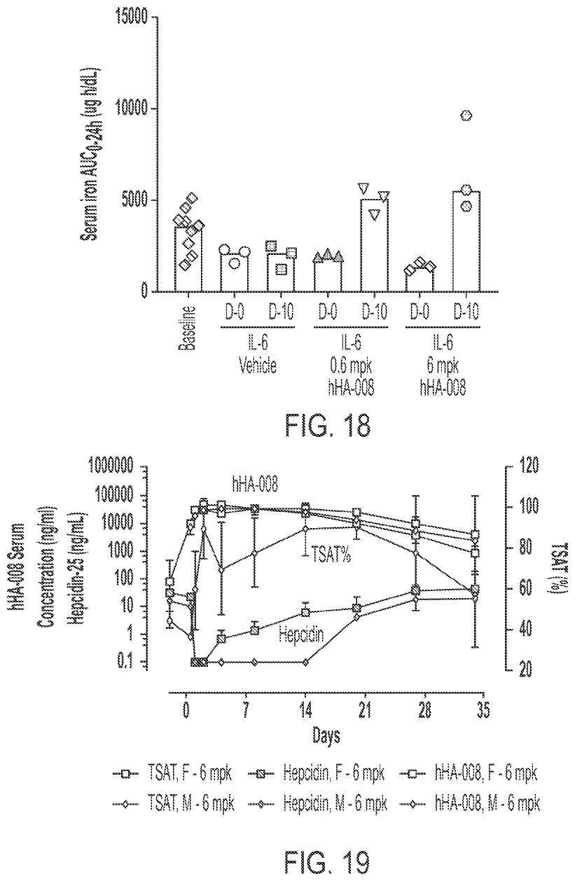

Prevention of iron suppression by hHA-008 following IL-6 challenge was investigated in this study. On Day 0, three groups of cynos (N=3/group) were challenged with 6×10⁴ international units (IU) IL-6/kg subcutaneously. On Day 3 each group of 3 animals received 0 (vehicle), 0.6 or 6 mg/kg of hHA-008 intravenously, respectively. On Day 10 all groups received a second challenge of 6×10⁴ IU IL-6/kg subcutaneously. Blood was collected from all cynos to evaluate serum iron level at 48 hours and 24 hours prior to each IL-6 challenge, and 2, 4, 8 and 24 hours after each IL-6 dose. The average baseline value of the samples taken 48 and 24 hours prior to each IL-6 dose were used as DO value. The results showed that hHA-008 was effective in preventing IL-6-induced serum iron suppression in a dose-dependent manner in cynomolgus monkeys (FIG. 18).

### Example 10: Subcutaneous Injection of hHA-008 in Sprague-Dawley Rats

Sprague-Dawley Rats (6 females and 6 males) were injected with hHA-008 subcutaneously at 6 mg/kg (mpk). pharmacokinetics (SC PK) and pharmacodynamics (SC PD) were evaluated 35 days after dosing. Serum mean pharmacokinetics parameters (e.g., Cₘₐₓ, Tₘₐₓ, t_{1/2}, and AUC_{0-inf}) were shown in Table 26:

TABLE 26
Serum Mean Pharmacokinetic (PK) Parameters of hHA-008 Following
a Single SC Administration in Sprague-Dawley Rats

| | C_{MAX} | T_{MAX} | T_{1/2} | AUC_{0-INF} |
|---|---|---|---|---|
| DOSE | (MG/ML) | (DAYS) | (DAYS) | (HR*MG/ML) |
| (MG/KG) | (MEAN ± SD) | (MEAN ± SD) | (MEAN ± SD) | (MEAN ± SD) |
| 6 | 37.6 ± 6.02 | 3.8 ± 0.6 | 4.3 ± 1.4 | 13,357 ± 1,437.4 |

Bioavailability of hHA-008 after subcutaneous administration was similar to bioavailability of hHA-008 after intravenous administration at 6 mg/kg, as evidenced by that subcutaneous injection bioavailability (SC bioavailability) of hHA-008 was about 84.7% of intravenous injection bioavailability of hHA-008 (IV bioavailability) (SC bioavailability=SC DN_AUC_{0-inf}/IV DN_AUC_{0-inf}×100; DN: Dose normalized). Further, the time to reach maximum concentration (Cₘₐₓ) was 3.6-4 days after SC administration with a much lower Cₘₐₓ as compared to the Cₘₐₓ after IV administration.

At similar dose level, the PD response between the IV or SC was indistinguishable: serum hHA-008 concentration became saturated 1-2 days after dosing, remained elevated through at least 21 days and the decline in PD response (e.g., Hepcidin-25 concentration and TSAT %) was consistent with the decrease of hHA-008 serum concentration (FIG. 19).

### Example 11. Subcutaneous Dose Administration in Cynomolgus Monkeys

PK/PD of subcutaneous hHA-008 treatment were also evaluated in Cynomolgus (cynos). Cynos were injected with hHA-008 subcutaneously at 0.3 mpk, 0.6 mpk, 1 mpk and 6 mpk.

In the 6 mpk dosing group, 2 male and 1 female cynos received a single subcutaneous injection of hHA-008 at, and blood samples were collected for 61 days. Cynos received IV injection of hHA-008 at 6 mpk were used for comparison.

In the 0.3 mpk, 0.6 mpk, and 1.0 mpk groups, each group included 3 male cynos that received a single subcutaneous injection of hHA-008. Blood samples were collected for 27 days. Cynos received the same dose of hHA-008 by IV were used for comparison for each group.

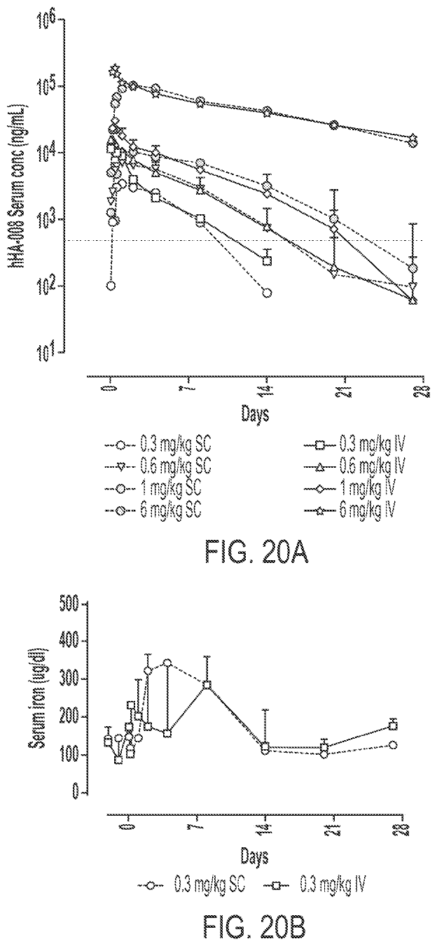

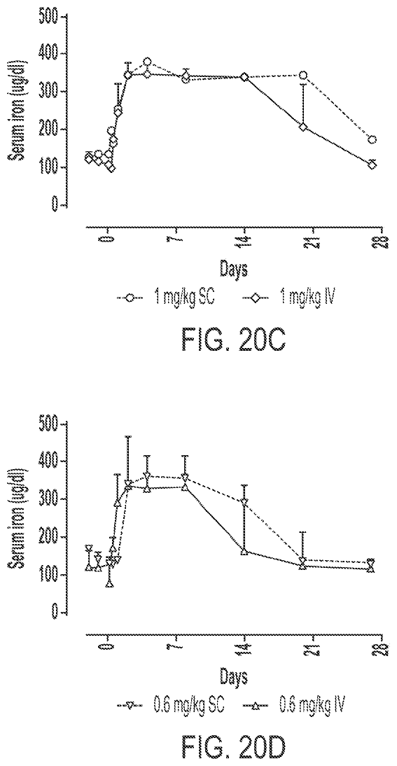

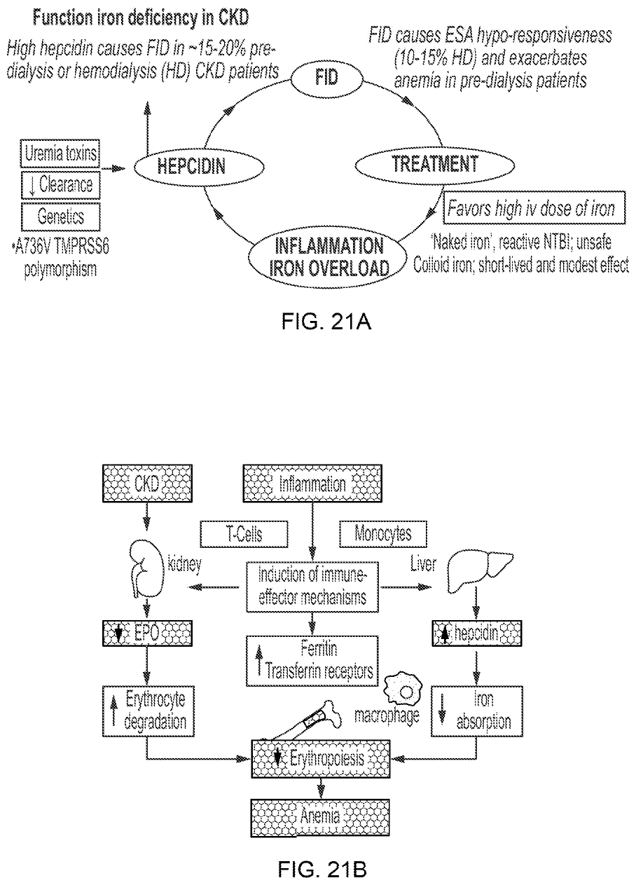

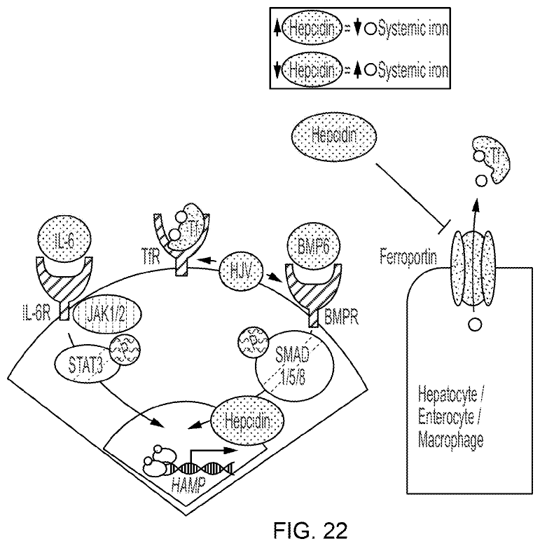

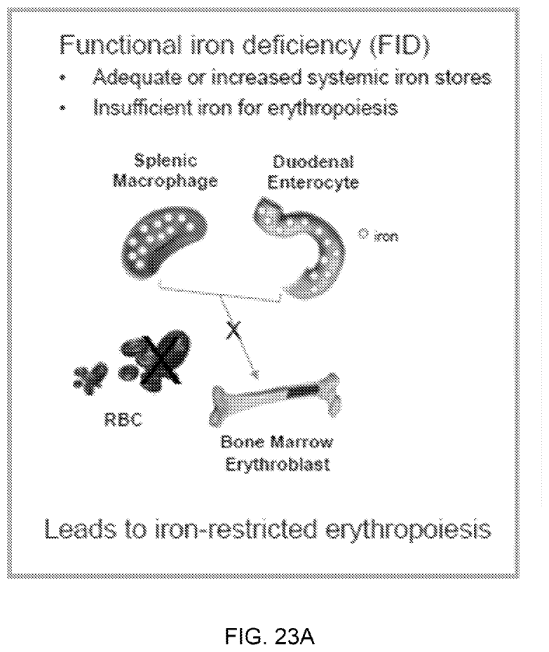

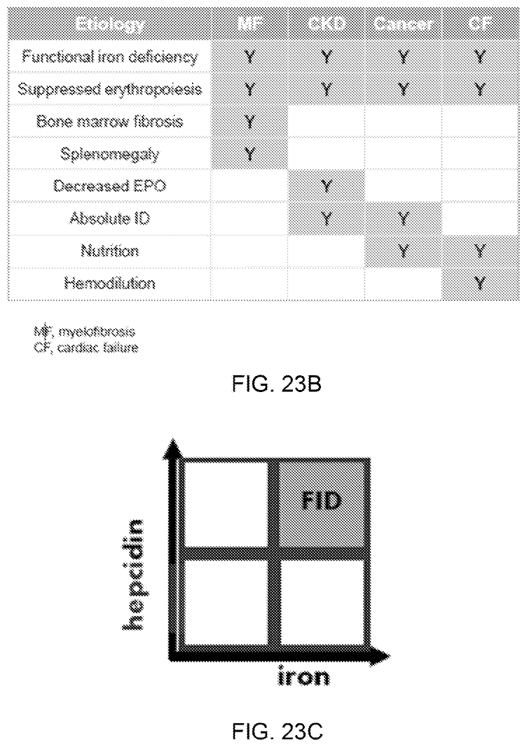

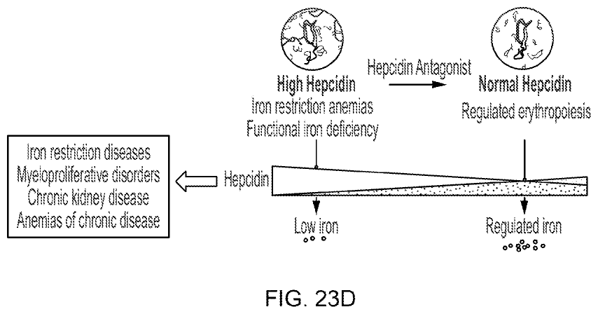

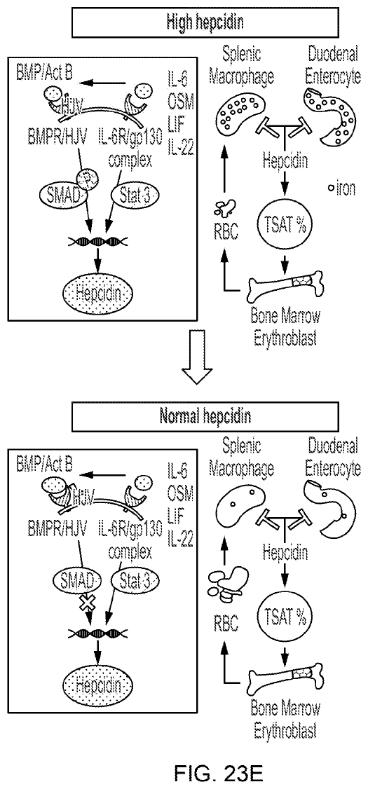

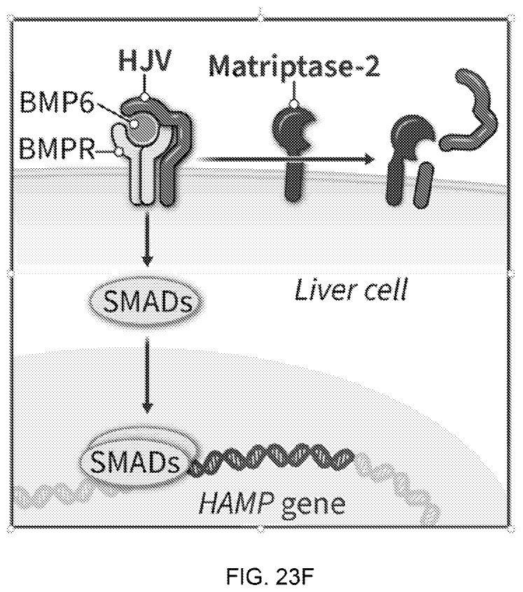

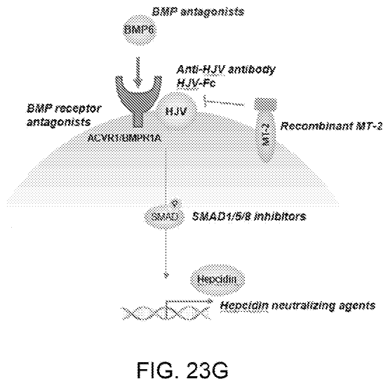

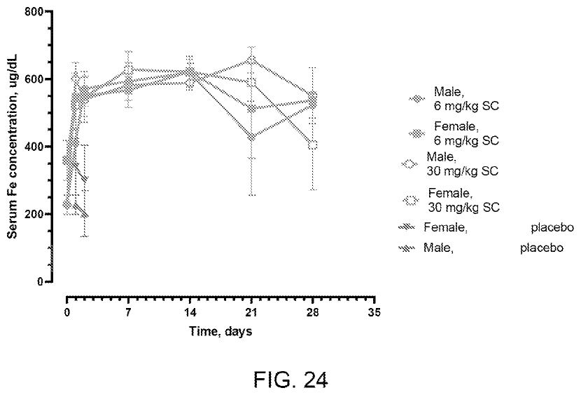

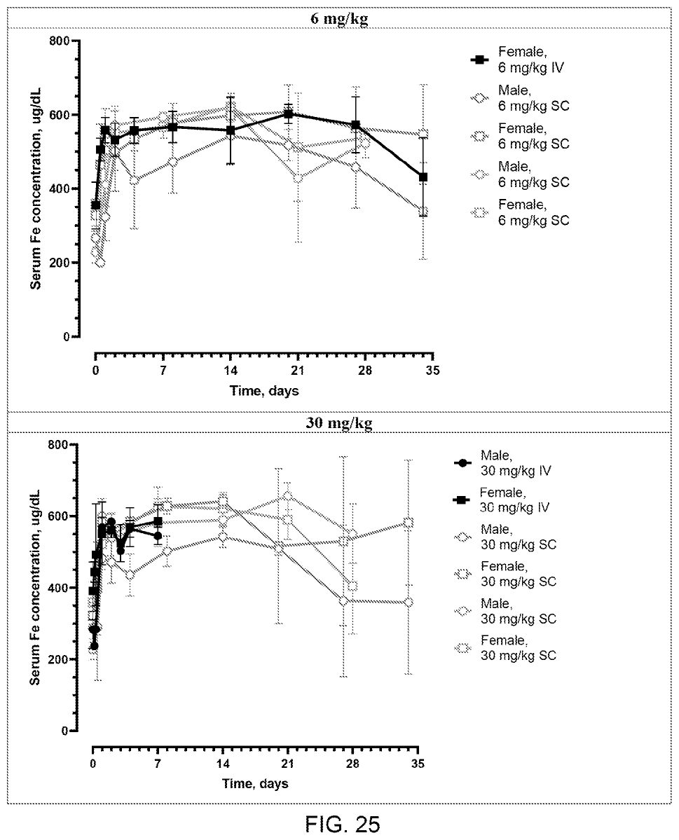

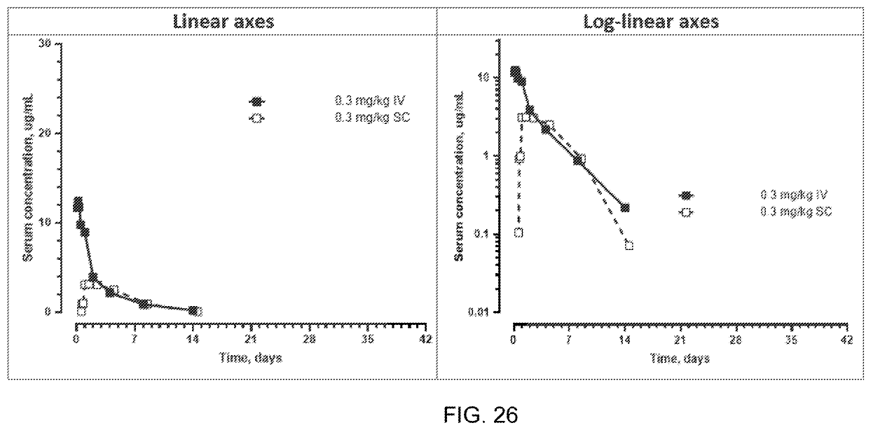

After SC administration of hHA-008 in cynomolgus monkeys, bioavailability was high and increased with dose levels (55.0% at 0.3 mpk, 81.9% at 0.6 mpk, 89.5% at 1 mpk and 100% at 6 mg/kg), when compared to animals injected at the same doses via IV. Tₘₐₓ was reached rapidly after dosing in a dose dependent manner (1.33 to 2.67 days). Table 27 shows serum mean Pharmacokinetic (PK) parameters of hHA-008 following a single SC administration in cynos. hHA-008 serum concentration-time profiles became indistinguishable between SC injection and IV injection 4 days after administration (FIG. 20A).

TABLE 27
Serum Mean Pharmacokinetic (PK) Parameters of hHA-
008 Following a Single SC Administration in Cynos

| | | | | AUC_{0-inf}* | Bioavailability |
|---|---|---|---|---|---|
| Dose | Cₘₐₓ (μg/mL) | Tₘₐₓ (day) | t_{1/2} (day) | (hr*μg/mL) | compared to |
| (mg/kg) | (mean ± SD) | (mean ± SD) | (mean ± SD) | (mean ± SD) | IV |
| 0.3 | 3.21 ± 0.72 | 1.33 ± 0.58 | 1.69 ± 0.18 | 496.5 ± 81.29 | 55% |
| 0.6 | 7.2 ± 0.48 | 1.33 ± 0.58 | 2.82 ± 0.2 | 1417 ± 234 | 81.9% |
| 1 | 10.5 ± 1.76 | 2 ± 0 | 3.3 ± 1.43 | 2942 ± 300 | 89.5% |
| 6 | 102 ± 15.4 | 2.67 ± 1.15 | 7.95 ± 1.68 | 34439 ± 2,528 | 100% |

*extrapolation from AUC₀₋ₗₐₛₜ was less than 3%.

The PD response after SC dose was variable between animals, relatively proportional to the dose-level, and similar to the PD response observed after IV dosing at 0.1 mpk, 0.6 mpk, and 1.0 mpk. For all animals, the PD response was functionally maximal (TSAT>80% and hepcidin-25<LOQ) between 1-2 days after dosing. The return of the PD markers (e.g., serum iron) to baseline levels was consistent with the decline in hHA-008 serum concentrations (FIGS. 20B-20D). At 6 mpk, serum iron concentrations in both SC injected group and IV injected group were too saturated to reflect the change (data not shown).

### Example 12. Comparison of Intravenous and Subcutaneous Administration Pharmacokinetics and Pharmacodynamics

### Background

Toxicology studies to support initial intravenous (IV) clinical studies with HHA-008 in healthy volunteers included a 1 month repeat dose IV study in rats and monkey. The subcutaneous (SC) route of administration in healthy volunteers was also studied. Hence, SC tolerability and pharmacokinetics/pharmacodynamic study (PKPD) study in rats to support evaluation of this dosage route in clinical trials. hHA-008 is defined by an antibody having a VH region comprising an amino acid sequence of SEQ ID NO: 38, and a VL region comprising an amino acid sequence of SEQ ID NO: 39.

In terms of study criteria, eligibility of healthy volunteers (or healthy controls) may be separated by sex (on average, healthy women exhibit hemoglobin levels of 1 g/dL lower than healthy men, <10.5 g/dL and <11.5 g/dL, respectively). Subjects may be eligible if their serum hepcidin or urine hepcidin levels are greater than the median of healthy volunteers.

A nonclinical IV safety program was completed following recommendations and guidance to support IV dosing in initial clinical trials. To support evaluation of SC administration in humans, a single dose tolerability and PKPD study was completed in rats. Since the toxicologic profile of hHA-008 following IV administration was well defined, and systemic SC exposure was known to be similar to IV dosing based on previous PKPD studies (including those of Examples 10 and 11, above), it was determined that local tolerance following SC administration was the only additional data required prior to implementing subcutaneous dosing in healthy volunteers in the hHA-008-101 study.

The SC tolerability and PKPD study in rats included a local tolerance phase and a PKPD phase (Table 4).

TABLE 4
Design of the Local Tolerance and Pharmacokinetic/Pharmacodynamic
Phases in Rats Administered Single Subcutaneous
Doses of hHA-008

| | Dose | Dose | | |
|---|---|---|---|---|
| Dose Level | Volume | Concentration | Number of Animals | |
| (mg/kg) | (mL/kg) | (mg/mL) | Males | Females |

Local Tolerance Phase

| | | | | |
|---|---|---|---|---|
| 0^{a} | 5 | 0 | 10 | 10 |
| 400^{a} | 5 | 80^{b} | 10 | 10 |
| 400^{c} | 5 | 80^{b} | 10 | 10 |

Pharmacokinetic/Pharmacodynamic Phase

| | | | | |
|---|---|---|---|---|
| 0 | 5 | 0 | 5 | 5 |
| 6 | 5 | 1.2 | 9 | 9 |
| 30 | 5 | 6 | 9 | 9 |

^{a}Scheduled necropsy 48 hours postdose.
^{b}hHA-008 administered as neat stock solution of 79.6 mg/mL.
^{c}Schedule necropsy on Day 28.

In the local tolerance phase, male and female CD® [CRL:CD® (SD)] rats were administered a single SC dose of vehicle (buffer) or hHA-008 at 400 mg/kg. The hHA-008 dose utilized was the high dose (maximum feasible dose) in a previous 1 month IV study, which also was the no observed adverse effect level (NOAEL) in that study. Ten animals/sex from the control and hHA-008 treated groups were euthanized 48 hours postdose, and 10 hHA-008 treated animals were euthanized 28 days postdose.

In the PKPD phase, male and female rats were administered a single SC dose of vehicle or hHA-008 (6 or 30 mg/kg) and evaluated for 28 days. Dose levels were selected based on previous PKPD studies and the 1 month repeat dose IV study, which demonstrated significant, prolonged pharmacodynamic effects of hHA-008.

The following endpoints and parameters were evaluated: mortality, clinical signs, body weight, body weight gain, food consumption, ophthalmology, clinical chemistry, PD parameters (serum iron, transferrin, transferrin saturation [TSAT], unsaturated iron binding capacity [UIBC], and total iron binding capacity [TIBC]), and serum hHA-008 concentrations (limited sampling in local tolerance phase). Organ weights, gross pathology, and microscopic examination of injection sites and Prussian Blue stained sections of liver, spleen, and heart were assessed in the local tolerance phase only. Samples for potential analysis of anti drug antibodies were collected from PKPD animals prior to termination.

All animals survived to scheduled termination. There were no drug related body weight, body weight gain, food consumption, or ophthalmic changes. Changes in clinical laboratory parameters in 5 females at 30 mg/kg in the current study (Table 5) were carefully scrutinized since in a previous 1 month IV study in monkeys, increases in ALT along with liver changes (cytoplasmic alteration of hepatocytes and/or hepatocyte necrosis) at ≥30 mg/kg were considered adverse. Mild increases in aspartate aminotransferase (AST; up to 3 fold), alanine aminotransferase (ALT; up to 2 fold), and/or alkaline phosphatase (ALP; up to 40%) were noted in a few females at 30 mg/kg compared to the upper end of the concurrent control range on Day 28 (Table 5). These changes were not considered adverse or hHA-008 related, as similar changes were not observed at 400 mg/kg 48 hours or 28 days postdose (Table 6). Additionally, there were no drug related changes in these parameters in a previous 1 month IV study in rats at dose levels up to 400 mg/kg.

TABLE 5
Clinical Laboratory Parameters on Day 28 in Female Rats Administered
a Single Subcutaneous Dose of hHA-008 at 30 mg/kg

| Animal | Dose | AST | ALT | ALP |
|---|---|---|---|---|
| (Sex) | (mg/kg) | (U/L) | (U/L) | (U/L) |
| 1 (F) | 30 | 88 | 59 | 139 |
| 3 (F) | 30 | 134 | 34 | 58 |
| 6 (F) | 30 | 117 | 32 | 116 |
| 7 (F) | 30 | 315 | 81 | 155 |
| 9 (F) | 30 | 104 | 33 | 88 |

| | | | |
|---|---|---|---|
| Concurrent Control Values | | | |
| Mean ± Standard Deviation | 86.8 ± 13.4 | 34.2 ± 4.7 | 94.8 ± 10.9 |
| Range-65-100-28-41-86-111 | | | |

Bolded values above the concurrent control range.
ALP = Alkaline Phosphatase;
ALT = Alanine aminotransferase;
AST = Aspartate aminotransferase

TABLE 6
Clinical Laboratory Parameters in Female Rats 48 Hours or 28
Days After a Single Subcutaneous Dose of hHA-008 at 400 mg/kg

| Dose | | | | |
|---|---|---|---|---|
| (mg/kg) | | AST (U/L) | ALT (U/L) | ALP (U/L) |
| 0 | Mean ± Standard | 71.8 ± 11.1 | 26.7 ± 4.2 | 141.3 ± 33.0 |
| (48 hours) | Deviation | | | |
| | Range 61-87-25-38-11-198 | | | |
| 400 | Mean ± Standard | 70.8 ± 7.0 | 27.6 ± 1.03 | 122.3 ± 19.3 |
| (48 hours) | Deviation | | | |
| | Range-67-84-26-42-35-242 | | | |
| 400 | Mean ± Standard | 73.0 ± 7.1 | 38.6 ± 8.3 | 114.5 ± 34.0 |
| (Day 28) | Deviation | | | |
| | Range-57-96-30-40-78-197 | | | |

ALP = Alkaline Phosphatase;
ALT = Alanine aminotransferase;
AST = Aspartate aminotransferase

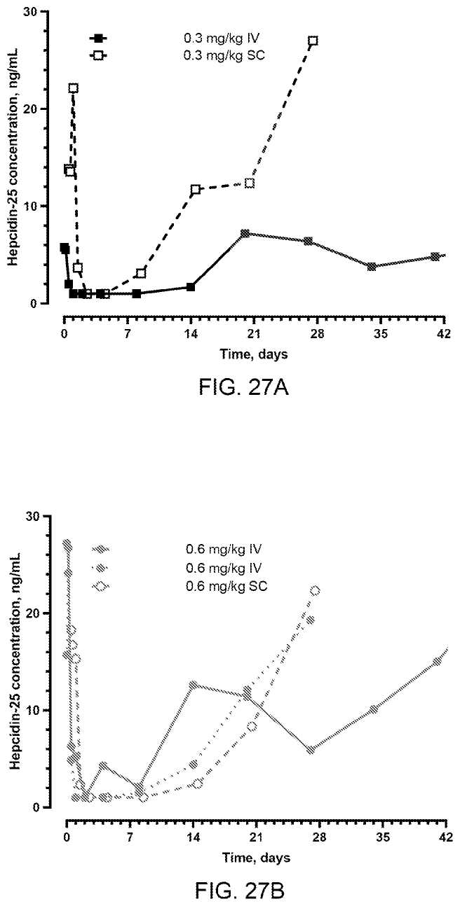

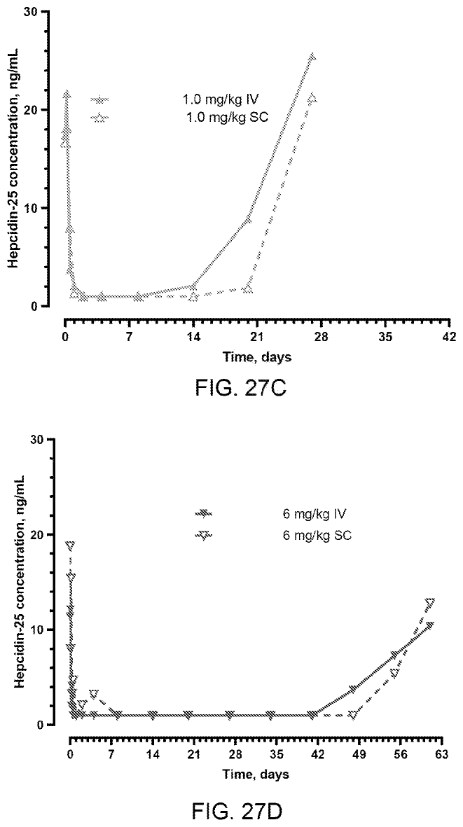

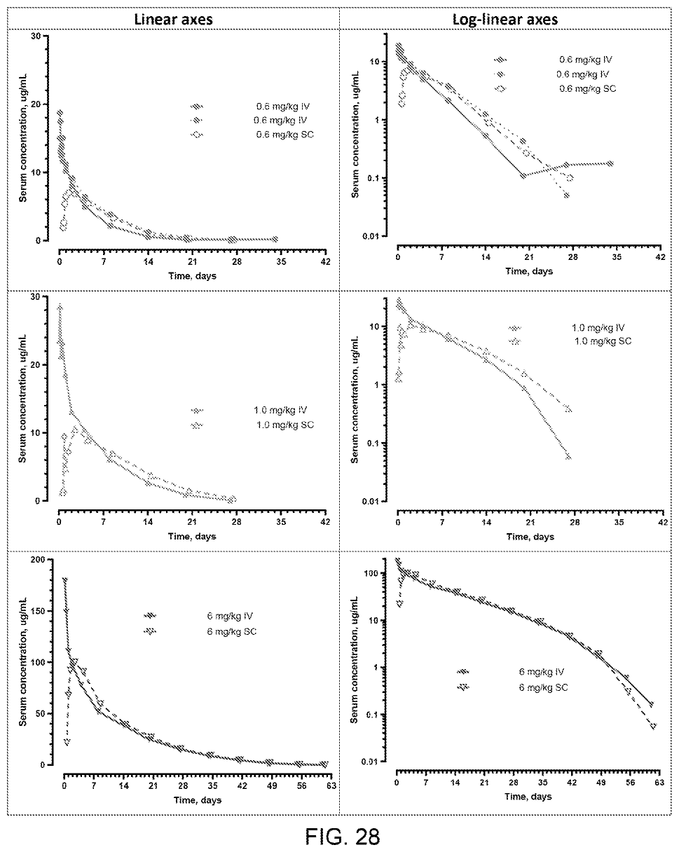

Serum iron levels were increased to near maximal levels in the PKPD phase at 6 and 30 mg/kg at 24 hours postdose, and remained elevated at those levels for 28 days. In the local tolerability phase, serum iron was increased to near maximal levels at 400 mg/kg at 24 hours postdose; on Day 28, serum iron remained near maximal levels in females but was slightly lower than maximal levels in males (FIGS. 27A-27D).

Serum TSAT were increased in the PKPD phase in both sexes at 6 and 30 mg/kg at 24 hours postdose and remained elevated at these levels through Day 28 except in females at 30 mg/kg, where TSAT was similar to controls at 24 hours postdose. In the local tolerability phase, TSAT was increased at 24 hours postdose in both sexes at 400 mg/kg and in females on Day 28. Partial or complete recovery noted in most males at 400 mg/kg on Day 28.

No significant changes were noted in TIBC or transferrin concentrations across dose levels or time.

hHA-008-related microscopic findings involving pigment (iron based on Prussian Blue staining) were present in the liver and spleen of males and females receiving 400 mg/kg at 48 hours post-dose and on Day 28 (Table 7).

TABLE 7
Iron Pigment in Spleen and Liver of Rats Administered
a Single Subcutaneous Dose of HHA-008

| | Male | | | Female | | |
|---|---|---|---|---|---|---|
| | Dose (mg/kg): | | | | | |
| | 0 | 400 | 400 | 0 | 400 | 400 |
| | Time of Euthanasia (Postdose) | | | | | |
| | 48 hr | 48 hr | Day 28 | 48 hr | 48 hr | Day 28 |
| | Number of Animals Examined: | | | | | |
| | 10 | 10 | 10 | 10 | 10 | 10 |
| Spleen | | | | | | |
| Pigment decreased | | | | | | |
| Marked | 0 | 4 | 2 | 0 | 2 | 6 |
| Liver | | | | | | |
| Pigment, hepatocellular, | | | | | | |
| increased | | | | | | |
| Minimal | 0 | 1 | 2 | 0 | 8 | 1 |
| Mild | 0 | 0 | 4 | 0 | 0 | 2 |
| Moderate | 0 | 0 | 4 | 0 | 0 | 6 |
| Marked | 0 | 0 | 0 | 0 | 0 | 1 |

Liver pigment (iron based on the Prussian Blue sections) was minimally to markedly increased in affected animals compared to controls. Pigment was primarily periportal and stain intensity varied between and within the two liver lobes evaluated. The overall incidence and average severity grade of liver pigment were greater in the Day 28 animals than in the 48 hours post-dose animals. The magnitude (intensity) of splenic pigment (iron based on the Prussian Blue sections) was markedly decreased in affected animals compared to controls. No hHA-008-related changes in Prussian Blue stained sections of heart were observed.

Under the conditions of this study, the pigment-related changes in the spleen and liver were not considered adverse. Microscopic alterations in Prussian Blue staining (iron) in the spleen (decreased pigment) and liver (increased pigment) correlated with increases in serum iron concentration and TSAT and decreases in UIBC. Changes in these parameters in this study were similar to those observed in a previous 1-month rat study. Based on these findings, the NOAEL in this study was 400 mg/kg.

HHA-008 toxicokinetic parameters were similar in males and females and increased with increasing dose at 6 and 30 mg/kg (Table 9). Limited sampling at 400 mg/kg precluded calculation of toxicokinetic parameters, but concentrations at the sampling times included in this study were similar to comparable timepoints in a previous 1-month IV study.

TABLE 9
hHA-008 Toxicokinetic Parameters in Rats
Administered a Single Subcutaneous

| Dose | Cmax | Tₘₐₓ | AUC_{0-648 h} | AUC_{0-∞} | T_{1/2} |
|---|---|---|---|---|---|
| (mg/kg) | (μg/mL) | (hr) | (hr · μg/mL) | (hr · μg/mL) | (days) |
| 6 | 40.4 | 72 | 10,900 | 11,800 | 7.2 |
| 30 | 182 | 96 | 70,200 | 76,400 | 6.8 |

AUC_{0-648 h} = Area under the concentration-time curve from time 0 to 648 hours postdose; AUC_{0-∞} = Area under the concentration-time curve from time 0 to infinity; Cₘₐₓ = Maximum observed concentration; T_{1/2} = Half-life; Tₘₐₓ = Time of maximum observed concentration.

### Overall Conclusion:

The toxicologic, pharmacodynamic, and toxicokinetic profiles in rats administered a single SC dose were nearly identical to those observed following IV dosing in a previous 1-month IV study, and there were no hHA-008 injection site changes. These data, along with previous IV studies, support SC administration in humans. Based on these findings, the NOAEL in this study was 400 mg/kg.

### Example 13. PKPD Comparison of IV and SC Administration in Rat and Monkey

To support a clinical development program, the PK and PD of HHA-008 were characterized in Sprague-Dawley rats and cynomolgus monkeys following IV and SC administration.

### Rats

No single study provided direct SC and IV comparisons in rats. However, identical SC and IV doses were given across studies. Table 10 provides a comparison of PD parameters for the 6 mg/kg and 30 mg/kg doses given in different studies. Results of the comparison show that hHA-008 exposures and half-lives are similar after IV and SC administrations for each of these two dose levels.

TABLE 10
Pharmacokinetic comparisons of hHA-008 after 6
mg/kg or 30 mg/kg doses given to rats as either
an intravenous (IV) or subcutaneous (SC) dose

| | Dose | | | | | Half- |
|---|---|---|---|---|---|---|
| | level, | Cₘₐₓ, | Tₘₐₓ, | AUC_{7 d}, | AUC_{inf}, | life, |
| Route | mg/kg | ug/mL | h | ug*h/mL | ug*h/mL | d |
| IV | 6 | 148 | 2 | — | 15600 | 5.9 |
| SC | 6 | 37.6 | 96 | — | 13400 | 4.3 |
| SC | 6 | 40.3 | 72 | — | 11700 | 7.2 |
| IV | 30 | 778 | 0.083 | 42000 | 65600 | 9.9 |
| IV | 30 | 468 | 2 | 39100 | — | — |
| SC | 30 | 195 | 96 | — | 95800 | 10.5 |
| SC | 30 | 193 | 48 | — | 72700 | 8.3 |

Pharmacokinetic parameters: Cₘₐₓ = maximum concentration; Tₘₐₓ = time after dose of Cmax occurrence; AUC_{7 d} = area under the curve, from time of dosing to 7 days hence; AUC_{inf} = area under the curve from time of dosing to infinity; Half-life = half-life. Cited Cₘₐₓ, AUC, and half-life values represent mean values from male and female rats combined, with the exception of one study, which only dosed female rats. Tₘₐₓ values are medians. All values represent 3 significant digits except half-life, which is accurate to one decimal place.

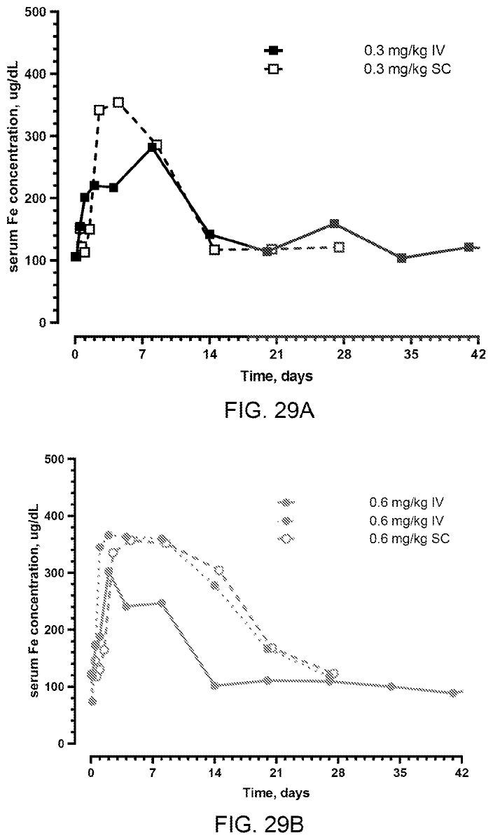

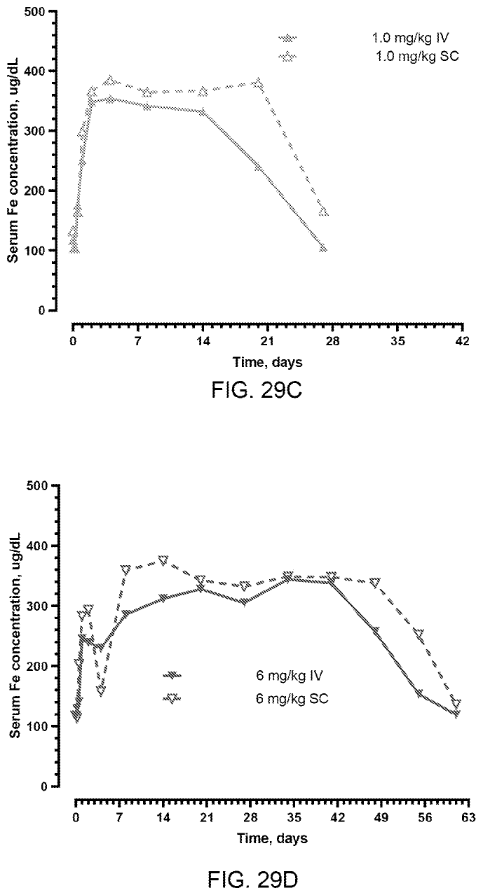

Each of the studies summarized in the table above also monitored serum iron (Fe) concentrations as a PD marker. FIGS. 29A-29D shows the serum Fe response for both the 6 mg/kg and 30 mg/kg doses given as either a SC or IV dose. Like the PK, the serum Fe response appeared to be nearly identical between the SC and IV administrations for each dose level.

### Monkeys

Identical SC and IV doses were given to cynomolgus monkeys across several studies as single dose administrations (see Table 11).

TABLE 11
Single dose HHA-008 monkey studies used for IV and SC
comparisons of pharmacokinetic and pharmacodynamic data

| Administration | |
|---|---|
| route | Dose, mg/kg |
| IV | 6 |
| SC | 6 |
| IV | 0.1, 0.3, 0.6 |
| IV | 0.6, 1.0 |
| SC | 0.3, 0.6, 1.0 |

### Pharmacokinetic Comparisons

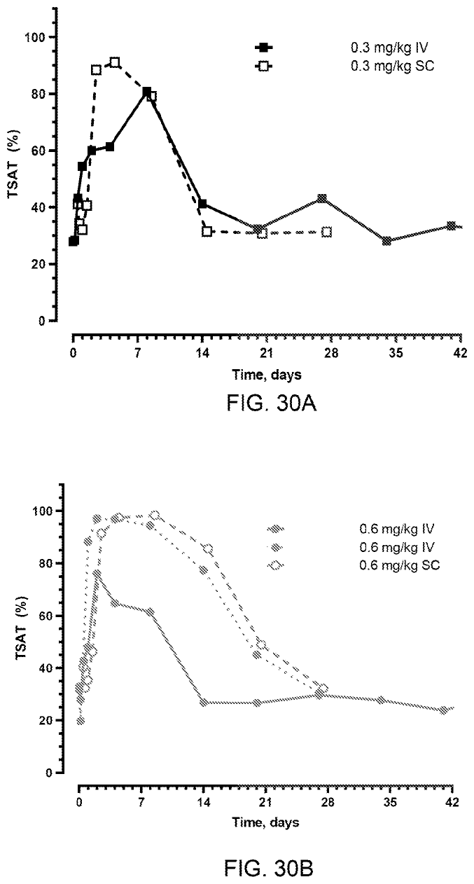

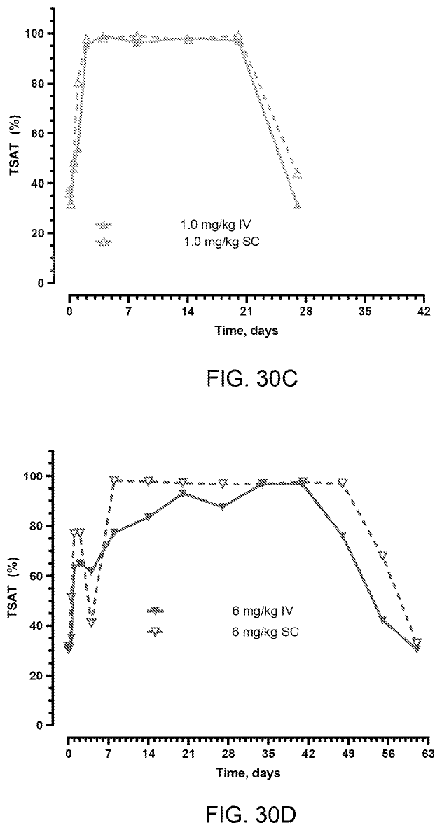

FIGS. 30A-30D shows comparisons between the SC and IV pharmacokinetics across all doses on both linear and log-linear axes. Note that after absorption is complete from SC administration serum hHA-008 concentrations are comparable between the IV and SC administrations at all dose levels.

Table 12 provides a comparison of pertinent PK parameters between the IV and SC dose administrations. Except for Cₘₐₓ and Tₘₐₓ, the results suggest comparable AUC₀₋ₗₐₛₜ and AUC_{inf} values for the 0.6, 1.0, and 6.0 mg/kg dose levels.

TABLE 12
Mean (±SD) hHA-008 pharmacokinetics after a single subcutaneous
or intravenous dose at 0.3, 0.6, 1.0 or 6.0 mg/kg to cynomolgus monkeys

| Dose | | Cₘₐₓ | | Tₘₐₓ | t_{1/2} | | AUC₀₋ₗₐₛₜ | | AUC_{0-inf} | |
|---|---|---|---|---|---|---|---|---|---|---|
| (mg/kg) | Route | (μg/mL) | | (day) | (day) | | (hr*μg/mL) | | (hr*μg/mL) | |
| 0.3 | IV | 12.2 | (1.39) | 0.13¹ | 2.9 | (0.6) | 895 | (330) | 908 | (81.29) |
| | SC | 3.21 | (0.72) | 1.33 (0.58) | 1.69 | (0.18) | 492.2 | (79.7) | 496.5 | (81.29) |
| 0.6 | IV | 15.0 | (3.34) | 0.13¹ | 3.2 | (0.3) | 1488 | (210) | 1493 | (209) |
| | IV | 16.6 | (1.59) | 0.14 | 2.43 | (0.35) | 1943 | (384) | 1951 | (378) |
| | SC | 7.2 | (0.48) | 1.33 (0.58) | 2.82 | (0.2) | 1405 | (234) | 1417 | (234) |
| 1.0 | IV | 26.9 | (3.45) | 0.14 | 2.99 | (1.44) | 3223 | (676) | 3298 | (791) |
| | SC | 10.5 | (1.76) | 2 (0) | 3.3 | (1.43) | 2884 | (226) | 2942 | (300) |
| 6.0 | IV | 179 | (10.8) | 0.25 | 3.0 | (0.51) | 34887 | (3770) | 34900 | (3763) |
| | SC | 102 | (15.4) | 2.67 (1.15) | 7.95 | (1.68) | 34420 | (2518) | 34439 | (2528) |

¹Sampling schedule across animals differed to reduce the blood volume collection within each animal. Tmax value represents an average across animals.

### Pharmacodynamic Comparisons

PD responses have also been monitored across all monkey studies allowing comparisons between SC and IV dosing. Three PD measurements—hepcidin-25 concentrations, serum Fe concentrations, and % transferrin saturation (TSAT %)—with SC vs. IV profiles for each in FIGS. 27A-27D, 28, and 29A-29D respectively.

These comparisons show a similar PD response profile between SC and IV administrations for all dose levels. In addition, the peak response (i.e., an Eₘₐₓ) for each PD parameter is independent of the administration route. Furthermore, the PD profiles closely follow the pharmacokinetic behavior, with a reduction from the maximum response when hHA-008 concentrations drop below 1-2 μg/mL. This is consistent with the known pharmacology of hHA-008.

In summary, PKPD profiles following IV and SC administrations in both rat and monkey were nearly superimposable suggesting that response to SC and IV doses in humans should be comparable. The Cₘₐₓ and tₘₐₓ values for the SC dose should be reduced and prolonged, respectively, compared to IV, but the hHA-008 concentrations beyond Cₘₐₓ are not expected to differ between the two routes. Thus, no difference should be expected between dose selection and dosing frequency for SC and IV hHA-008 administration. In this ongoing clinical study, healthy volunteers received a 7 mg IV dose in the 1^{st} cohort. hHA-008 single SC dose in HVs will begin at the next planned dose of 14 mg, providing that the safety profiles from the 7 mg IV dose cohort in this study supports continued dose escalation.

### Example 14. Clinical Protocol

### Background

Hepcidin-induced iron restriction results from a variety of inflammatory stimuli and can cause anemia of inflammation. Hemojuvelin (HJV) is glycosylphosphatidylinositol-anchored membrane protein expressed in iron-loading tissues (liver, heart, muscle) that binds bone morphogenetic protein (BMP) family receptors as a central regulator of hepcidin expression. hHA-008 is a mAb developed to target HJV and block binding of HJV with BMP receptors to reduce hepcidin production and treat anemia of inflammation. hHA-008 decreased Hamp mRNA in a mouse model of heat-killed Brucella abortus-triggered inflammation and improved hemoglobin in a rat model of peptidoglycan-polysaccharide 10-induced anemia of inflammation. hHA-008 also showed dose-proportional decreases in circulating hepcidin-25 and increase in serum iron in a non-human primate model of IL-6-induced hypoferremia.

### Study Design and Methods

Eligible subjects are healthy females of non-reproductive potential and males 18-65 years old, without known hemochromatosis risk factors and with normal baseline red blood cell parameters, normal serum iron, normal total iron binding capacity (TIBC), morning transferrin saturation (TSAT)<30% (or <25%), and serum ferritin ≥30 ng/mL.

Subjects were randomly allocated in each cohort 6:2, hHA-008: placebo in a blinded fashion. Unblinding of treatment allocation occurred after an assessment of each cohort, prior to initiating the next dose level. The starting dose level was 7 mg total dose (0.1 mg/kg), and the escalation planned to follow a 2-fold increase. Escalation may be up to a 42 mg total dose (0.6 mg/kg). Escalation may be up to a 56 mg total dose (0.8 mg/kg). Escalation may be up to a 60 mg or a 90 mg total dose. Any of these doses may be administered monthly or semi-monthly. Subjects were evaluated for a minimum of 28 days and then until circulating hHA-008 was no longer detectable or 10 weeks, whichever was sooner.

Primary endpoints were safety and tolerability, as assessed through adverse events, vital signs, physical examinations, ECGs, and clinical lab testing. Secondary and exploratory endpoints included pharmacokinetics, serum iron, hepcidin-25, transferrin, TIBC, TSAT, red blood cell indices, and ferritin. Endpoints were summarized using descriptive statistics.

### Example 15. Reduction of Hepcidin and Improved Anemia in a Rat Model of Chronic Kidney Disease

### Background

A lead anti-hemojuvelin antibody, identified as Anti-HJV Ab, is a humanized monoclonal antibody that is currently in Phase I clinical studies. HJV is a pathway-specific coreceptor for bone morphogenetic protein (BMP) signaling that regulates hepcidin expression and iron metabolism that is encoded by the HFE2 gene. Loss of function mutations in HFE2 in humans cause profound reductions in hepcidin synthesis and severe iron overload. Consequently, it is hypothesized that pharmacological reduction of HJV activity will lead to reduction of hepcidin synthesis and increased iron availability. This hypothesis has been confirmed in studies of Anti-HJV Ab in rodents, non-human primates and healthy human volunteers. In addition, anemia is a common complication in patients with CKD and has been associated with multiple adverse outcomes in this population. In CKD hepcidin is increased because of both reduced renal clearance and increased synthesis. This increase is believed to be a central contributor to the development of anemia by reducing the availability of iron from systemic iron stores and by reducing dietary iron absorption. The following studies were conducted to evaluate the effects of Anti-HJV Ab in an animal model of CKD anemia.

### Study Design and Methods:

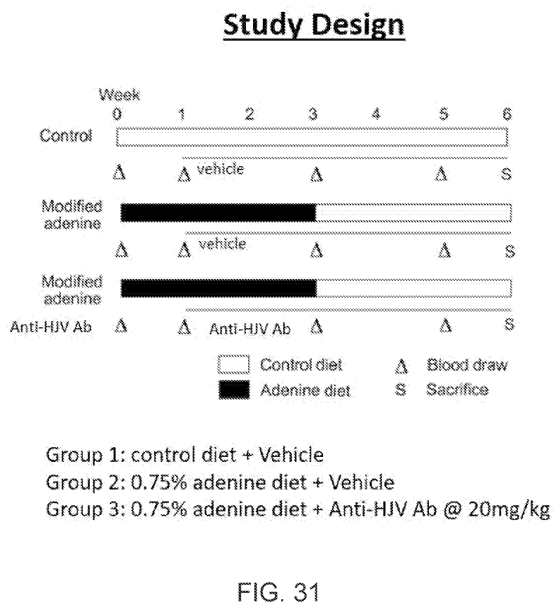

The effect of Anti-HJV Ab on improving anemia was evaluated in an adenine-induced CKD rat model. In this study, and consistent with a previous report (Sun et al, Nephrol Dial Transplant. 2013; 28:1733-1743), renal impairment was induced by giving Sprague Dawley rats a diet containing 0.75% adenine for 3 weeks to induce kidney injury and then switched to a control diet for the remainder of the study. Anti-HJV Ab (20 mg/kg) or vehicle (n=5/group) was administered intravenously once per week starting on Day 7 and continuing to Day 35. An additional group of rats (n=5/group) was fed with the control diet the entire duration of the study and dosed intravenously with vehicle. Rats were euthanized on Day 42. Blood was collected on Days 0, 7, 21, 35, and 42 for evaluation of hematology parameters, as well as serum iron, hepcidin and complete blood count (CBC). Serum was further analyzed for creatinine, urea nitrogen, TIBC, and erythropoietin (EPO) levels. Liver tissue was also collected on Day 43 for evaluation of hepcidin gene (HAMP) mRNA expression level. A schematic of this study design is shown in FIG. 31.

### Results

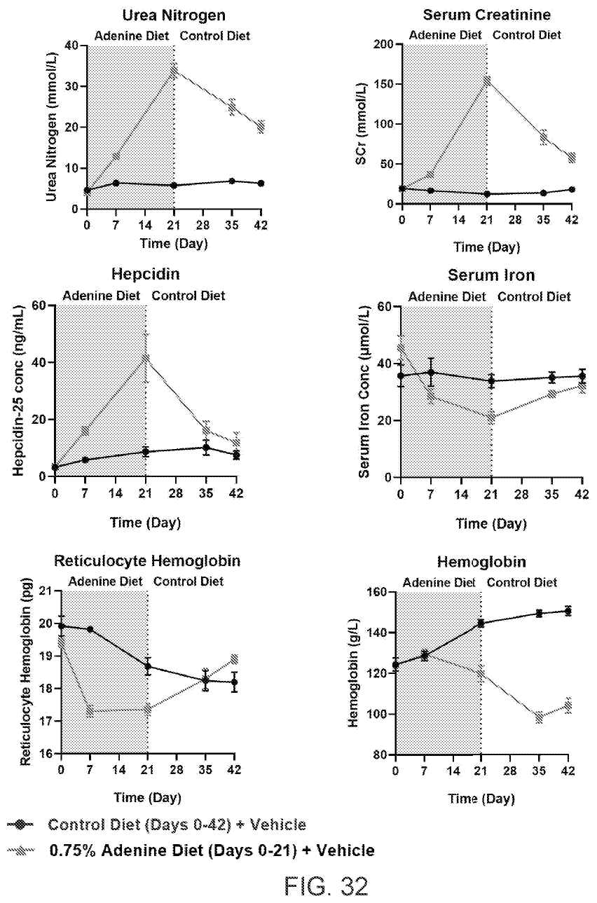

As shown in FIG. 32, rats on the 0.75% adenine diet developed kidney dysfunction, as evidenced by the marked increase in serum creatinine and urea nitrogen. These rats also developed increased hepcidin and anemia (e.g., decreases in serum iron and hemoglobin), presenting a hypochromic microcytic profile as a consequence of reduced iron availability. These rats appeared to tolerate Anti-HJV Ab treatment well, as Anti-HJV Ab at a dose of 20 mg/kg did not affect body weight.

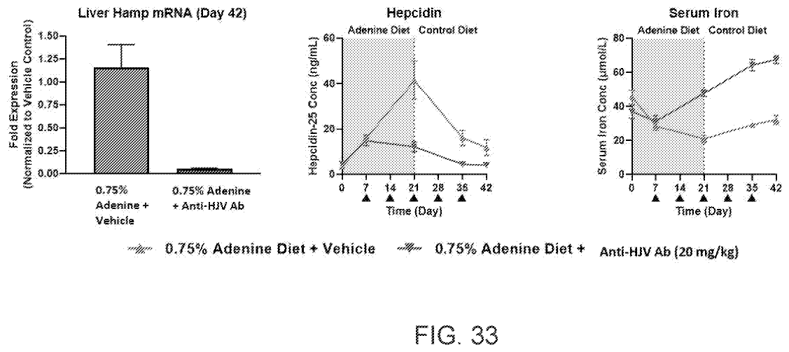

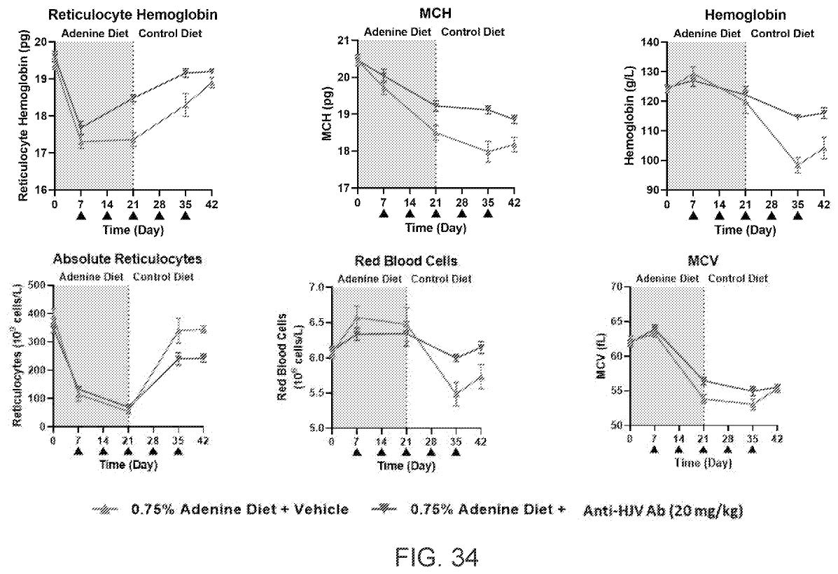

Consistent with the proposed mechanism of action of blocking the formation of the BMP/BMPR/HJV complex, Anti-HJV Ab reduced HAMP gene expression in the liver as measured at the end the study, which led to reduced serum hepcidin-25 and increased serum iron concentration (FIG. 33). As shown in FIG. 34, Anti-HJV Ab treatment improved anemia in CKD rats. Cellular hemoglobinization, measured as reticulocyte hemoglobin (RET-He), was increased. The maximum improvement in hemoglobin concentration following Anti-HJV Ab treatment, compared to the vehicle group, was 17 g/L in the rat CKD model.

### CONCLUSIONS

Treatment with Anti-HJV Ab at a dose that reduces hepcidin gene expression is able to increase iron availability and substantially prevents the reduction in hemoglobin that is seen in animals that develop adenine-induced renal impairment. These results support the development of Anti-HJV Ab for the treatment of patients with CKD anemia.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including.” “carrying.” “having.” “containing.” “involving.” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A method of treating a subject having anemia, wherein the subject is identified as having a level of glomerular filtration rate (GFR) of less than 90 mL/min per 1.73 m, the method comprising:
administering to the subject and effective amount of a hemojuvelin antagonist.

2-73. (canceled)


SEQUENCE LISTING (131 sequences)

SEQ ID NO: 1 - protein, 5 aa, synthetic construct
  REGION 1..5: note = Synthethic Polypeptide
NYGMN

SEQ ID NO: 2 - protein, 17 aa, synthetic construct
  REGION 1..17: note = Synthethic Polypeptide
MIYYDSSEKHYADSVKG

SEQ ID NO: 3 - protein, 6 aa, synthetic construct
  REGION 1..6: note = Synthethic Polypeptide
GTTPDY

SEQ ID NO: 4 - protein, 16 aa, synthetic construct
  REGION 1..16: note = Synthethic Polypeptide
RSSQSLETSDGDTFLE

SEQ ID NO: 5 - protein, 7 aa, synthetic construct
  REGION 1..7: note = Synthethic Polypeptide
EVSTRFS

SEQ ID NO: 6 - protein, 9 aa, synthetic construct
  REGION 1..9: note = Synthethic Polypeptide
FQVTHDPMT

SEQ ID NO: 7 - protein, 115 aa, synthetic construct
  REGION 1..115: note = Synthethic Polypeptide
EVQLVESGGGLVQPGGSLRLSCAASGFTFSNYGMNWIRQAPGKGLEWIGMIYYDSSEKHYADSVKGRFTISRDNSKNTLYLQMNSLRAEDTAVYYCAKGTTPDYWGQGTMVTVSS

SEQ ID NO: 8 - protein, 112 aa, synthetic construct
  REGION 1..112: note = Synthethic Polypeptide
DVVLTQSPLSLPVTLGQPASISCRSSQSLETSDGDTFLEWFQQRPGQSPRLLIYEVSTRFSGVPDRFSGSGSGTDFTLKISRVEAEDVGVYYCFQVTHDPMTFGQGTKLEIK

SEQ ID NO: 9 - protein, 5 aa, synthetic construct
  REGION 1..5: note = Synthethic Polypeptide
YYGMN

SEQ ID NO: 10 - protein, 17 aa, synthetic construct
  REGION 1..17: note = Synthethic Polypeptide
MIYYDSSDKHYADSVKG

SEQ ID NO: 11 - protein, 6 aa, synthetic construct
  REGION 1..6: note = Synthethic Polypeptide
GTTPDV

SEQ ID NO: 12 - protein, 6 aa, synthetic construct
  REGION 1..6: note = Synthethic Polypeptide
GTTPDK

SEQ ID NO: 13 - protein, 6 aa, synthetic construct
  REGION 1..6: note = Synthethic Polypeptide
GSTPDY

SEQ ID NO: 14 - protein, 16 aa, synthetic construct
  REGION 1..16: note = Synthethic Polypeptide
RSSQSLESSDGDTFLE

SEQ ID NO: 15 - protein, 16 aa, synthetic construct
  REGION 1..16: note = Synthethic Polypeptide
RSSQSLEESDGYTFLE

SEQ ID NO: 16 - protein, 16 aa, synthetic construct
  REGION 1..16: note = Synthethic Polypeptide
RSSQSLEDSDGGTFLE

SEQ ID NO: 17 - protein, 16 aa, synthetic construct
  REGION 1..16: note = Synthethic Polypeptide
RSSQSLEESDGYTFLH

SEQ ID NO: 18 - protein, 7 aa, synthetic construct
  REGION 1..7: note = Synthethic Polypeptide
EVSNRFS

SEQ ID NO: 19 - protein, 7 aa, synthetic construct
  REGION 1..7: note = Synthethic Polypeptide
DVSTRFS

SEQ ID NO: 20 - protein, 7 aa, synthetic construct
  REGION 1..7: note = Synthethic Polypeptide
DVSERFS

SEQ ID NO: 21 - protein, 7 aa, synthetic construct
  REGION 1..7: note = Synthethic Polypeptide
DVSSRFS

SEQ ID NO: 22 - protein, 7 aa, synthetic construct
  REGION 1..7: note = Synthethic Polypeptide
AVSHRFS

SEQ ID NO: 23 - protein, 7 aa, synthetic construct
  REGION 1..7: note = Synthethic Polypeptide
EVSHRFS

SEQ ID NO: 24 - protein, 9 aa, synthetic construct
  REGION 1..9: note = Synthethic Polypeptide
MQVTHDPLT

SEQ ID NO: 25 - protein, 9 aa, synthetic construct
  REGION 1..9: note = Synthethic Polypeptide
FQVTHDPVT

SEQ ID NO: 26 - protein, 9 aa, synthetic construct
  REGION 1..9: note = Synthethic Polypeptide
FQATYDPLT

SEQ ID NO: 27 - protein, 9 aa, synthetic construct
  REGION 1..9: note = Synthethic Polypeptide
FQATHDPLT

SEQ ID NO: 28 - protein, 9 aa, synthetic construct
  REGION 1..9: note = Synthethic Polypeptide
FQATHDPVT

SEQ ID NO: 29 - protein, 9 aa, synthetic construct
  REGION 1..9: note = Synthethic Polypeptide
FQATHDPLS

SEQ ID NO: 30 - protein, 112 aa, synthetic construct
  REGION 1..112: note = Synthethic Polypeptide
DVVLTQSPLSLPVTLGQPASISCRSSQSLETSDGDTFLEWFQQRPGQSPRLLIYEVSSRFSGVPDRFSGSGSGTDFTLKISRVEAEDVGVYYCMQVTHDPLTFGQGTKLEIK

SEQ ID NO: 31 - protein, 112 aa, synthetic construct
  REGION 1..112: note = Synthethic Polypeptide
DVVLTQSPLSLPVTLGQPASISCRSSQSLETSDGDTFLEWFQQRPGQSPRLLIYEVSNRFSGVPDRFSGSGSGTDFTLKISRVEAEDVGVYYCFQVTHDPVTFGQGTKLEIK

SEQ ID NO: 32 - protein, 112 aa, synthetic construct
  REGION 1..112: note = Synthethic Polypeptide
DVVLTQSPLSLPVTLGQPASISCRSSQSLESSDGDTFLEWFQQRPGQSPRLLIYDVSTRFSGVPDRFSGSGSGTDFTLKISRVEAEDVGVYYCFQVTHDPVTFGQGTKLEIK

SEQ ID NO: 33 - protein, 112 aa, synthetic construct
  REGION 1..112: note = Synthethic Polypeptide
DVVLTQSPLSLPVTLGQPASISCRSSQSLEESDGYTFLEWFQQRPGQSPRLLIYDVSERFSGVPDRFSGSGSGTDFTLKISRVEAEDVGVYYCFQATYDPLTFGQGTKLEIK

SEQ ID NO: 34 - protein, 115 aa, synthetic construct
  REGION 1..115: note = Synthethic Polypeptide
EVQLVESGGGLVQPGGSLRLSCAASGFTFSYYGMNWIRQAPGKGLEWIGMIYYDSSEKHYADSVKGRFTISRDNSKNTLYLQMNSLRAEDTAVYYCAKGTTPDYWGQGTMVTVSS

SEQ ID NO: 35 - protein, 112 aa, synthetic construct
  REGION 1..112: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLTFGQGTKLEIK

SEQ ID NO: 36 - protein, 115 aa, synthetic construct
  REGION 1..115: note = Synthethic Polypeptide
EVQLVESGGGLVQPGGSLRLSCAASGFTFSNYGMNWIRQAPGKGLEWIGMIYYDSSDKHYADSVKGRFTISRDNSKNTLYLQMNSLRAEDTAVYYCAKGTTPDVWGQGTMVTVSS

SEQ ID NO: 37 - protein, 112 aa, synthetic construct
  REGION 1..112: note = Synthethic Polypeptide
DVVLTQSPLSLPVTLGQPASISCRSSQSLEESDGYTFLHWFQQRPGQSPRLLIYEVSNRFSGVPDRFSGSGSGTDFTLKISRVEAEDVGVYYCFQATHDPVTFGQGTKLEIK

SEQ ID NO: 38 - protein, 115 aa, synthetic construct
  REGION 1..115: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSS

SEQ ID NO: 39 - protein, 112 aa, synthetic construct
  REGION 1..112: note = Synthethic Polypeptide
DVVLTQSPLSLPVTLGQPASISCRSSQSLEESDGYTFLHWFQQRPGQSPRLLIYEVSTRFSGVPDRFSGSGSGTDFTLKISRVEAEDVGVYYCFQATHDPLTFGQGTKLEIK

SEQ ID NO: 40 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggaatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggctcgacccccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - protein, 112 aa, synthetic construct
  REGION 1..112: note = Synthethic Polypeptide
DVVLTQSPLSLPVTLGQPASISCRSSQSLADSDGDTFLHWFQQRPGQSPRLLIYAVSHRFSGVPDRFSGSGSGTDFTLKISRVEAEDVGVYYCFQATHDPVTFGQGTKLEIK

SEQ ID NO: 42 - protein, 115 aa, synthetic construct
  REGION 1..115: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDKWGQGTMVTVSS

SEQ ID NO: 43 - protein, 112 aa, synthetic construct
  REGION 1..112: note = Synthethic Polypeptide
DVVLTQSPLSLPVTLGQPASISCRSSQSLEESDGYTFLEWFQQRPGQSPRLLIYEVSHRFSGVPDRFSGSGSGTDFTLKISRVEAEDVGVYYCFQATHDPLTFGQGTKLEIK

SEQ ID NO: 44 - protein, 115 aa, synthetic construct
  REGION 1..115: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGSTPDYWGQGTMVTVSS

SEQ ID NO: 45 - protein, 112 aa, synthetic construct
  REGION 1..112: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLSFGQGTKLEIK

SEQ ID NO: 46 - protein, 330 aa, synthetic construct
  REGION 1..330: note = Synthethic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 107 aa, Homo sapiens [sequence omitted]

SEQ ID NO: 48 - protein, 330 aa, synthetic construct
  REGION 1..330: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 49 - protein, 7 aa, synthetic construct
  REGION 1..7: note = Synthethic Polypeptide
EVSSRFS

SEQ ID NO: 50 - protein, 16 aa, synthetic construct
  REGION 1..16: note = Synthethic Polypeptide
RSSQSLADSDGDTFLH

SEQ ID NO: 51 - protein, 445 aa, synthetic construct
  REGION 1..445: note = Synthethic Polypeptide
EVQLVESGGGLVQPGGSLRLSCAASGFTFSNYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 219 aa, synthetic construct
  REGION 1..219: note = Synthethic Polypeptide
DVVLTQSPLSLPVTLGQPASISCRSSQSLETSDGDTFLEWFQQRPGQSPRLLIYEVSTRFSGVPDRFSGSGSGTDFTLKISRVEAEDVGVYYCFQVTHDPMTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 219 aa, synthetic construct
  REGION 1..219: note = Synthethic Polypeptide
DVVLTQSPLSLPVTLGQPASISCRSSQSLETSDGDTFLEWFQQRPGQSPRLLIYEVSSRFSGVPDRFSGSGSGTDFTLKISRVEAEDVGVYYCMQVTHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 219 aa, synthetic construct
  REGION 1..219: note = Synthethic Polypeptide
DVVLTQSPLSLPVTLGQPASISCRSSQSLETSDGDTFLEWFQQRPGQSPRLLIYEVSNRFSGVPDRFSGSGSGTDFTLKISRVEAEDVGVYYCFQVTHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 219 aa, synthetic construct
  REGION 1..219: note = Synthethic Polypeptide
DVVLTQSPLSLPVTLGQPASISCRSSQSLESSDGDTFLEWFQQRPGQSPRLLIYDVSTRFSGVPDRFSGSGSGTDFTLKISRVEAEDVGVYYCFQVTHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 219 aa, synthetic construct
  REGION 1..219: note = Synthethic Polypeptide
DVVLTQSPLSLPVTLGQPASISCRSSQSLEESDGYTFLEWFQQRPGQSPRLLIYDVSERFSGVPDRFSGSGSGTDFTLKISRVEAEDVGVYYCFQATYDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 445 aa, synthetic construct
  REGION 1..445: note = Synthethic Polypeptide
EVQLVESGGGLVQPGGSLRLSCAASGFTFSYYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 219 aa, synthetic construct
  REGION 1..219: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 445 aa, synthetic construct
  REGION 1..445: note = Synthethic Polypeptide
EVQLVESGGGLVQPGGSLRLSCAASGFTFSNYGMNWIRQAPGKGLEWIGMIYYDSSDKHYADSVKGRFTISRDNSKNTLYLQMNSLRAEDTAVYYCAKGTTPDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 219 aa, synthetic construct
  REGION 1..219: note = Synthethic Polypeptide
DVVLTQSPLSLPVTLGQPASISCRSSQSLEESDGYTFLHWFQQRPGQSPRLLIYEVSNRFSGVPDRFSGSGSGTDFTLKISRVEAEDVGVYYCFQATHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 219 aa, synthetic construct
  REGION 1..219: note = Synthethic Polypeptide
DVVLTQSPLSLPVTLGQPASISCRSSQSLEESDGYTFLHWFQQRPGQSPRLLIYEVSTRFSGVPDRFSGSGSGTDFTLKISRVEAEDVGVYYCFQATHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 64 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggacagcgacggaggcacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgtttccaagccacccacgaccccctcagcttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 65 - protein, 219 aa, synthetic construct
  REGION 1..219: note = Synthethic Polypeptide
DVVLTQSPLSLPVTLGQPASISCRSSQSLADSDGDTFLHWFQQRPGQSPRLLIYAVSHRFSGVPDRFSGSGSGTDFTLKISRVEAEDVGVYYCFQATHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDKWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 219 aa, synthetic construct
  REGION 1..219: note = Synthethic Polypeptide
DVVLTQSPLSLPVTLGQPASISCRSSQSLEESDGYTFLEWFQQRPGQSPRLLIYEVSHRFSGVPDRFSGSGSGTDFTLKISRVEAEDVGVYYCFQATHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGSTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 219 aa, synthetic construct
  REGION 1..219: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaattggattagacaagcgcccggaaaaggattggaatggataggaatgatatactacgatagctcagaaaaacattatgccgactcagttaaaggaagatttacaatatcaagagacaatagcaaaaacacactttatcttcaaatgaattcactgcgagccgaggatacagcagtctattattgcgcaaaaggaacaacaccagattattggggtcaaggaacaatggtaaccgtgtcaagc

SEQ ID NO: 71 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaattggattagacaagcgcccggaaaaggattggaatggataggaatgatatactacgatagctcagaaaaacattatgccgactcagttaaaggaagatttacaatatcaagagacaatagcaaaaacacactttatcttcaaatgaattcactgcgagccgaggatacagcagtctattattgcgcaaaaggaacaacaccagattattggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 72 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaacgagattctcaggagttccagacagattttcaggcagcggatccggcacagacttcacccttaaaattagcagagtagaagcagaagatgtaggagtgtattattgttttcaagtcacccacgaccccatgacgttcggacaaggaactaagctcgaaatcaaa

SEQ ID NO: 73 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaacgagattctcaggagttccagacagattttcaggcagcggatccggcacagacttcacccttaaaattagcagagtagaagcagaagatgtaggagtgtattattgttttcaagtcacccacgaccccatgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 74 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaagcagattctcaggagttccagacagattttcaggcagcggatccggcacagacttcacccttaaaattagcagagtagaagcagaagatgtaggagtgtattattgtatgcaagtcacccacgaccccctgaccttcggacaaggaactaagctcgaaatcaaa

SEQ ID NO: 75 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaagcagattctcaggagttccagacagattttcaggcagcggatccggcacagacttcacccttaaaattagcagagtagaagcagaagatgtaggagtgtattattgtatgcaagtcacccacgaccccctgaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 76 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagaccagcgacggagatacttttcttgagtggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagtcacccacgaccccgtcacgttcggacaaggaactaagctcgaaatcaaa

SEQ ID NO: 77 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagaccagcgacggagatacttttcttgagtggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagtcacccacgaccccgtcacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 78 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagtccagcgacggagacacttttcttgagtggttccaacaaagacccggacaaagcccacgcctgcttatttacgatgtatcaactagattctcaggagttccagacagattttcaggcagcggatccggcacagacttcacccttaaaattagcagagtagaagcagaagatgtaggagtgtattattgttttcaagtcacccatgaccccgtgaccttcggacaaggaactaagctcgaaatcaaa

SEQ ID NO: 79 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagtccagcgacggagacacttttcttgagtggttccaacaaagacccggacaaagcccacgcctgcttatttacgatgtatcaactagattctcaggagttccagacagattttcaggcagcggatccggcacagacttcacccttaaaattagcagagtagaagcagaagatgtaggagtgtattattgttttcaagtcacccatgaccccgtgaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 80 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggaaagcgacggatacacttttcttgaatggttccaacaaagacccggacaaagcccacgcctgcttatttacgatgtatcagaaagattctcaggagttccagacagattttcaggcagcggatccggcacagacttcacccttaaaattagcagagtagaagcagaagatgtaggagtgtattattgtttccaagcgacctacgaccccctcaccttcggacaaggaactaagctcgaaatcaaa

SEQ ID NO: 81 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggaaagcgacggatacacttttcttgaatggttccaacaaagacccggacaaagcccacgcctgcttatttacgatgtatcagaaagattctcaggagttccagacagattttcaggcagcggatccggcacagacttcacccttaaaattagcagagtagaagcagaagatgtaggagtgtattattgtttccaagcgacctacgaccccctcaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 82 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagttactatggaatgaactggattagacaagcgcccggaaaaggattggaatggataggcatgatatactacgacagctcggagaaacattatgccgactcagttaaaggaagatttacaatatcaagagacaatagcaaaaacacactttatcttcaaatgaattcactgcgagccgaggatacagcagtctattattgcgcaaaagggacgacgcccgattactggggtcaaggaacaatggtaaccgtgtcaagc

SEQ ID NO: 83 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggacagcgacggaggaacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgttttcaagcgacccacgaccccctgacgttcggacaaggaactaagctcgaaatcaaa

SEQ ID NO: 84 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagttactatggaatgaactggattagacaagcgcccggaaaaggattggaatggataggcatgatatactacgacagctcggagaaacattatgccgactcagttaaaggaagatttacaatatcaagagacaatagcaaaaacacactttatcttcaaatgaattcactgcgagccgaggatacagcagtctattattgcgcaaaagggacgacgcccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 85 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggacagcgacggaggaacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgttttcaagcgacccacgaccccctgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 86 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaactggataagacaagcgcccggaaaaggattggaatggataggcatgatatactacgacagctcggacaaacattatgccgactcagttaaaggaagatttacaatatcaagagacaatagcaaaaacacactttatcttcaaatgaattcactgcgagccgaggatacagcagtctattattgcgcaaaagggacgacgccggatgtatggggtcaaggaacaatggtaaccgtgtcaagc

SEQ ID NO: 87 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaagagagcgacggatacacttttcttcattggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagccacccacgaccccgtgacgttcggacaaggaactaagctcgaaatcaaa

SEQ ID NO: 88 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaactggataagacaagcgcccggaaaaggattggaatggataggcatgatatactacgacagctcggacaaacattatgccgactcagttaaaggaagatttacaatatcaagagacaatagcaaaaacacactttatcttcaaatgaattcactgcgagccgaggatacagcagtctattattgcgcaaaagggacgacgccggatgtatggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 89 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaagagagcgacggatacacttttcttcattggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagccacccacgaccccgtgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 90 - DNA, 345 bp, synthetic construct
  misc_feature 1..345: note = Synthethic Polynucleotide
  source 1..345: mol_type = other DNA
gaagtacagttggtagaaagcggcggaggacttgtgcaaccaggtggatctctcagactttcatgtgccgcaagcggttttacttttagtaactatggaatgaactgggttagacaagcgcccggaaaaggattggaatggataggcatgatatattacgacagctcggagaaacattatgccgactcagttaaaggaagatttacaatatcaagagacaatagcaaaaacacactttatcttcaaatgaattcactgcgagccgaggatacagcagtctattattgcgcaaaagggaccacccccgattactggggtcaaggaacaatggtaaccgtgtcaagc

SEQ ID NO: 91 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaagaaagcgacggatacacttttcttcactggttccaacaaagacccggacaaagcccacgcctgcttatttacgaggtatcaaccagattctcaggagttccagacagattttcaggcagcggatccggcacagacttcacccttaaaattagcagagtagaagcagaagatgtaggagtgtattattgtttccaagccacccacgaccccctgaccttcggacaaggaactaagctcgaaatcaaa

SEQ ID NO: 92 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcaaccaggtggatctctcagactttcatgtgccgcaagcggttttacttttagtaactatggaatgaactgggttagacaagcgcccggaaaaggattggaatggataggcatgatatattacgacagctcggagaaacattatgccgactcagttaaaggaagatttacaatatcaagagacaatagcaaaaacacactttatcttcaaatgaattcactgcgagccgaggatacagcagtctattattgcgcaaaagggaccacccccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 93 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaagaaagcgacggatacacttttcttcactggttccaacaaagacccggacaaagcccacgcctgcttatttacgaggtatcaaccagattctcaggagttccagacagattttcaggcagcggatccggcacagacttcacccttaaaattagcagagtagaagcagaagatgtaggagtgtattattgtttccaagccacccacgaccccctgaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 94 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcaaccaggtggatctctcagactttcatgtgccgcaagcggttttacttttagtaactatggaatgaactgggttagacaagcgcccggaaaaggattggaatggataggcatgatatattacgacagctcggagaaacattatgccgactcagttaaaggaagatttacaatatcaagagacaatagcaaaaacacactttatcttcaaatgaattcactgcgagccgaggatacagcagtctattattgcgcaaaagggaccacccccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggaccaactcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacaaccactacacgcagaagagcctctccctgtctccgggtaaatga

SEQ ID NO: 95 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgcggacagcgacggagatacttttcttcactggttccaacaaagacccggacaaagcccacgcctgcttatttacgcggtatcacacagattctcaggagttccagacagattttcaggcagcggatccggcacagacttcacccttaaaattagcagagtagaagcagaagatgtaggagtgtattattgtttccaagccacccatgaccccgtcacgttcggacaaggaactaagctcgaaatcaaa

SEQ ID NO: 96 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgcggacagcgacggagatacttttcttcactggttccaacaaagacccggacaaagcccacgcctgcttatttacgcggtatcacacagattctcaggagttccagacagattttcaggcagcggatccggcacagacttcacccttaaaattagcagagtagaagcagaagatgtaggagtgtattattgtttccaagccacccatgaccccgtcacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 97 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggcatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggcacgacgcccgataaatggggtcaaggaacaatggtaaccgtgtcaagc

SEQ ID NO: 98 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggagagcgacggatacacttttcttgagtggttccaacaaagacccggacaaagcccacgcctgcttatttacgaggtatcacatagattctcaggagttccagacagattttcaggcagcggatccggcacagacttcacccttaaaattagcagagtagaagcagaagatgtaggagtgtattattgtttccaagcgacccacgaccccctgacgttcggacaaggaactaagctcgaaatcaaa

SEQ ID NO: 99 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggcatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggcacgacgcccgataaatggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 100 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggagagcgacggatacacttttcttgagtggttccaacaaagacccggacaaagcccacgcctgcttatttacgaggtatcacatagattctcaggagttccagacagattttcaggcagcggatccggcacagacttcacccttaaaattagcagagtagaagcagaagatgtaggagtgtattattgtttccaagcgacccacgaccccctgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 101 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggaatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggctcgacccccgattactggggtcaaggaacaatggtaaccgtgtcaagc

SEQ ID NO: 102 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggacagcgacggaggcacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgtttccaagccacccacgaccccctcagcttcggacaaggaactaagctcgaaatcaaa

SEQ ID NO: 103 - protein, 330 aa, Homo sapiens [sequence omitted]

SEQ ID NO: 104 - protein, 19 aa, synthetic construct
  REGION 1..19: note = Synthethic Polypeptide
MEFGLSWLFLVAILKGVQC

SEQ ID NO: 105 - protein, 5 aa, synthetic construct
  REGION 1..5: note = Synthethic Polypeptide
  VARIANT 1: note = Xaa can be Asn or Tyr
XYGMN

SEQ ID NO: 106 - protein, 17 aa, synthetic construct
  REGION 1..17: note = Synthethic Polypeptide
  VARIANT 8: note = Xaa can be Glu or Asp
MIYYDSSXKHYADSVKG

SEQ ID NO: 107 - protein, 6 aa, synthetic construct
  REGION 1..6: note = Synthethic Polypeptide
  VARIANT 2: note = Xaa can be Thr or Ser
  VARIANT 6: note = Xaa can be Tyr, Val, or Lys
GXTPDX

SEQ ID NO: 108 - protein, 16 aa, synthetic construct
  REGION 1..16: note = Synthethic Polypeptide
  VARIANT 7: note = Xaa can be Ala or Glu
  VARIANT 8: note = Xaa can be Thr, Ser, Glu, or Asp
  VARIANT 12: note = Xaa can be Asp, Tyr, or Gly
  VARIANT 16: note = Xaa can be Glu or His
RSSQSLXXSDGXTFLX

SEQ ID NO: 109 - protein, 7 aa, synthetic construct
  REGION 1..7: note = Synthethic Polypeptide
  VARIANT 1: note = Xaa can be Glu, Asp, or Ala
  VARIANT 4: note = Xaa can Asn, Ser, Thr, Glu, or His
XVSXRFS

SEQ ID NO: 110 - protein, 9 aa, synthetic construct
  REGION 1..9: note = Synthethic Polypeptide
  VARIANT 1: note = Xaa can be Phe or Met
  VARIANT 3: note = Xaa can be Val or Ala
  VARIANT 5: note = Xaa can be His or Tyr
  VARIANT 8: note = Xaa can be Met, Leu, or Val
  VARIANT 9: note = Xaa can be Thr or Ser
XQXTXDPXX

SEQ ID NO: 111 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 114 - protein, 444 aa, synthetic construct
  REGION 1..444: note = Synthetic Polypeptide
EVQLVESGGGLVQPGGSLRLSCAASGFTFSNYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 444 aa, synthetic construct
  REGION 1..444: note = Synthetic Polypeptide
EVQLVESGGGLVQPGGSLRLSCAASGFTFSYYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 444 aa, synthetic construct
  REGION 1..444: note = Synthetic Polypeptide
EVQLVESGGGLVQPGGSLRLSCAASGFTFSNYGMNWIRQAPGKGLEWIGMIYYDSSDKHYADSVKGRFTISRDNSKNTLYLQMNSLRAEDTAVYYCAKGTTPDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 119 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDKWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGSTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 14 aa, synthetic construct
  REGION 1..14: note = Synthetic
SSPMALGANATATR

SEQ ID NO: 122 - protein, 14 aa, synthetic construct
  REGION 1..14: note = Synthetic
TSSPMALGANATAT

SEQ ID NO: 123 - protein, 364 aa, synthetic construct
  REGION 1..364: note = Synthetic
QCKILRCNAEYVSSTLSLRGGGSSGALRGGGGGGRGGGVGSGGLCRALRSYALCTRRTARTCRGDLAFHSAVHGIEDLMIQHNCSRQGPTPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSD

SEQ ID NO: 124 - protein, 21 aa, synthetic construct
  REGION 1..21: note = Synthetic
SSPMALGANATATRKLTIIFK

SEQ ID NO: 125 - protein, 30 aa, synthetic construct
  REGION 1..30: note = Synthetic
DNDFLFVQATSSPMALGANATTRKLTIIFK

SEQ ID NO: 126 - protein, 21 aa, synthetic construct
  REGION 1..21: note = Synthetic
QLCVGGCPPSQRLSRSERNRR

SEQ ID NO: 127 - protein, 12 aa, synthetic construct
  REGION 1..12: note = Synthetic
SQRLSRSERNRR

SEQ ID NO: 128 - protein, 426 aa, Homo sapiens
MGEPGQSPSPRSSHGSPPTLSTLTLLLLLCGHAHSQCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSDAGVPLSSATLLAPLLSGLFVLWLCIQ

SEQ ID NO: 129 - protein, 25 aa, synthetic construct
  REGION 1..25: note = Synthetic
DTHFPICIFCCGCCHRSKCGMCCKT

SEQ ID NO: 130 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 6 aa, synthetic construct
  MOD_RES 2: note = Phosphorylated tyrosine (Y)
PYLKTK